(19) United States
(12) Patent Application Publication
(10) Pub. No.: US 2025/0197491 A1
(43) Pub. Date: Jun. 19, 2025

(54) COMPOSITIONS AND METHODS FOR MODULATION OF MACROPHAGE ACTIVITY

(71) Applicant: Macomics Limited, Edinburgh (GB)

(72) Inventors: Luca Cassetta, Edinburgh (GB); Stephen Myatt, Cambridge (GB); Carola Ries, Cambridge (GB); Krzysztof B. Wicher, Cambridge (GB)

(73) Assignee: Macomics Limited, Edinburgh, EL (GB)

(21) Appl. No.: 18/845,659

(22) PCT Filed: Mar. 13, 2023
(86) PCT No.: PCT/GB2023/050592

§ 371 Date: Sep. 10, 2024

(30) Foreign Application Priority Data
Mar. 11, 2022 (GB) 2203384.9
Oct. 4, 2022 (GB) 2214614.6

(51) Int. Cl. C07K 16/28; G01N 33/68

(52) U.S. Cl. CPC C07K 16/2803; G01N 33/6854; C07K 2317/33; C07K 2317/34; C07K 2317/54; C07K 2317/55; C07K 2317/76; C07K 2317/92

(57) ABSTRACT

An antigen-binding protein capable of binding specifically to human LILRB1 and to human LILRB2, wherein the antigen-binding protein does not block the interaction of human LILRB1 with HLA-G tetramer and/or does not block the interaction of human LILRB2 with HLA-G tetramer, and the antigen-binding protein is capable of reprogramming macrophage.

### TECHNICAL FIELD

The invention relates to antigen-binding proteins, such as antibodies or antigen-binding fragments thereof, each of which is capable of binding specifically to both human LILRB1 and human LILRB2, and to their use for modifying the behavior of macrophage, particularly tumour-associated macrophage (TAM), for the treatment of diseases such as cancer and immunosuppressive conditions.

### BACKGROUND TO THE INVENTION

Tumours evolve as ecosystems consisting of tumour cells, stromal and infiltrating immune cells. Tumourigenesis is determined by the intrinsic properties of cancer cells and their interactions with components of the tumour microenvironment (TME). The poor prognostic outcome of a neoplastic lesion is determined not only by the type of mutation that has occurred, but also by the tumour stromal composition; the recruitment and activation of cytotoxic lymphocytes (e.g., CD8⁺ T cells) can suppress lethal tumour development, however, it is promoted by infiltration of tumour-associated macrophages (TAMs). TAMs are the major components of the tumoural ecosystem and correlate with clinical stage, poor overall survival, and reduced recurrence-free survival in different cancers.

Tumour-associated macrophages are among the most abundant immune cells in the TME. During the initial stages of tumour development, macrophages can either directly promote anti-tumour responses by killing tumour cells, or indirectly recruit and activate other immune cells. As genetic changes occur within the tumour, T helper 2 (TH2) cells begin to dominate the TME, TAMs begin to exhibit an immunosuppressive pro-tumour phenotype that promotes tumour progression, metastasis, and resistance to therapy. Thus, targeting TAMs has emerged as a strategy for cancer therapy. TAM-targeting strategies have focused on macrophage depletion and inhibition of their recruitment into the TME. However, these strategies have shown limited therapeutic efficacy, although trials are still underway with combination therapies. The fact that macrophages have the potential for anti-tumour activity has moved the TAM-targeting field toward the development of TAM-reprogramming strategies to support this anti-tumour immune response. Macrophages are potentially able to mount a robust anti-tumoural response as they can directly kill cancer cells if properly activated; they can support the adaptive immune response by presenting tumour antigens and by producing chemokines and cytokines that recruit and activate cytotoxic CD8⁺ T cells and NK cells. So, if these immune reactions are dominant in the tumour microenvironment, the development of malignant tumours will be suppressed. However, in many cases the tumour microenvironment alters macrophage functions from pro-inflammatory (i.e., tumouricidal) to trophic ones that resemble those of macrophages in the developing tissues. TAMs express immune suppressive receptors such as programmed cell death ligand 1 (PD-L1), that restrict CD8⁺ T cell activities upon binding of the immune-checkpoint receptors, programmed cell death protein 1 (PD1) and cytotoxic T-lymphocyte-associated protein 4 (CTLA4). As a result, these tumour-educated macrophages promote malignant tumour development instead of suppressing it.

The leukocyte Ig-like receptor subfamily B (LILRB) is a group of type I transmembrane glycoproteins with extracellular Ig-like domains and cytoplasmic immunoreceptor tyrosine-based inhibitory motifs (ITIMs). This group of ITIM-containing receptors includes LILRB1 (also known as CD85J, LIR1, ILT2), LILRB2 (also known as CD85D, LIR2, ILT4), LILRB3 (also known as CD85A, LIR3, ILT5), LILRB4 (also known as CD85K, LIR5, ILT3), and LILRB5 (also known as CD85C, LIR8). The biological functions and clinical significance of many of these LILRBs (ILTs) are still being investigated. There is also a LILR subfamily A (LILRA) that is a group of type I transmembrane glycoproteins with extracellular Ig-like domains and cytoplasmic immunoreceptor tyrosine-based activating motifs (ITAMs). The LILRA family includes 6 members: LILRA1 (also known as CD85I, LIR6), LILRA2 (also known as CD85H, LIR7, ILT1), LILRA3 (also known as CD85E, LIR4, ILT6, monocyte inhibitory receptor HM43/31), LILRA4 (also known as CD85G, ILT7), LILRA5 (also known as CD85F, LIR9, ILT11), and LILRA6 (also known as ILT8).

The LILR receptors have two to four extracellular Ig-like domains, they can be inhibitory “LILRB” or activating “LILRA”. Other than LILRA3, which is expressed only in soluble form, LILR are expressed as membrane-bound receptors. The inhibitory receptors (LILRB1 to B5) have long cytoplasmic tails within ITIM motifs. The activating receptors LILRA1 to A6, excluding A3, have short cytoplasmic tails and couple with ITAM-bearing Fc receptors. Individual LILR receptors are classified as group 1 (LILRB1, LILRB2, and LILRA1-3) or group 2 (LILRB3-5 and LILRA4-6) members, based on conservation of LILRB1 residues that are able to recognise human leukocyte antigen (HLA) class I molecules. Expression of individual LILR has been identified in immune cells, such as neutrophils, eosinophils, macrophages, dendritic cells, NK cells, B cells, T cells, and osteoclasts and non-immune cells such as endothelial cells and neurons. Human LILRB and mouse PIR-B can modulate the functions of ITAM-bearing receptors such as FcR, B cell receptor (BCR), and T cell receptor (TCR). LILR also modulate toll-like receptor (TLR) signaling and functions. LILR can exert immunomodulatory effects on a wide range of immune cells and can modulate a broad set of immune functions, including immune cell function, cytokine release, antibody production, and antigen presentation.

A subset of LILR recognise MHC class I (also known as HLA class I in humans). LILR family members can have both activating and inhibitory functions. The inhibitory receptors LILRB1 and LILRB2 show a broad specificity for classical and non-classical MHC alleles. LILRB1 exhibits preferential binding to β2-microglobulin-associated complexes. Unlike LILRB1, the binding of LILRB2 to HLA ligands does not require B2-microglobulin. The activating receptor LILRA1 and the soluble protein LILRA3 prefers β2-microglobulin-independent free heavy chains of MHC class I, and in particular HLA-C alleles.

Leukocyte immunoglobulin-like receptor subfamily B member 1 (LILRB1, CD85J, LIR1, ILT2) protein in humans is encoded by the LILRB1 gene found in a gene cluster at chromosomal region 19ql3.4. The receptor is expressed on immune cells where it binds to MHC class I molecules on antigen-presenting cells and transduces a negative signal that inhibits stimulation of an immune response. LILRB1 was also reported to be expressed in human gastric cancer cells and may enhance tumour growth. It is thought to control inflammatory responses and cytotoxicity to help focus the immune response and limit autoreactivity. Multiple transcript variants encoding different isoforms have been found for this gene.

Leukocyte immunoglobulin-like receptor subfamily B member 2 (LILRB2, CD85D, LIR2, ILT4) protein in humans is encoded by the LILRB2 gene found in a gene cluster at chromosomal region 19ql3.4. The receptor is expressed on immune cells where it binds to MHC class I molecules on antigen-presenting cells and transduces a negative signal that inhibits stimulation of an immune response. It is thought to control inflammatory responses and cytotoxicity to help focus the immune response and limit autoreactivity. The receptor is also expressed on human non-small cell lung cancer cells. Multiple transcript variants encoding different isoforms have been found for this gene.

Leukocyte immunoglobulin-like receptor subfamily A member 3 (LILRA3, CD85E, LIR4, ILT6, monocyte inhibitory receptor HM43/31) also known as CD85 antigen-like family member E (CD85e), immunoglobulin-like transcript 6 (ILT-6), and leukocyte immunoglobulin-like receptor 4 (LIR-4) protein in humans is encoded by the LILRA3 gene located within the leukocyte receptor complex on chromosome 19q13.4. Unlike many of its family, LILRA3 lacks a transmembrane domain. The function of LILRA3 is currently unknown; however, it is highly homologous to other LILR genes, and can bind human leukocyte antigen (HLA) class I. Therefore, if secreted, the LILRA3 may impair interactions of membrane-bound LILRs (such as LILRB1, an inhibitory receptor expressed on effector and memory CD8 T cells) with their HLA ligands, thus modulating immune reactions and influencing susceptibility to disease. Like the closely related LILRA1, LILRA3 binds to both normal and ‘unfolded’ free heavy chains of HLA class I, with a preference for free heavy chains of HLA-C alleles. LILRA3 also binds both classical HLA-A and non-classical HLA-G1, but with reduced affinities compared to either LILRB1 or LILRB2. The role of LILRA3 in cancer is poorly understood. However, mutations in LILRA3 have been reported in humans and are associated with immune disorders. For example, a homozygous 6.7-kb deletion of LILRA3 that reduces LILRA3 mRNA and protein expression is associated with Sjögren's syndrome, multiple sclerosis, and rheumatoid arthritis. It is therefore suggested that LILRA3 has a role in suppressing inflammation and immunity.

WO2020023268 (Amgen) describes combination therapies that comprise administering a first antibody, or antigen-binding fragment thereof, that binds PD-1, PD-L1, or PD-L2; and a second antibody, or antigen-binding fragment thereof, that binds LILRB1, LILRB2, or HLA-G.

Anti-LILRB1 antibodies disclosed include antibody clones MAB20171 and MAB20172 (R&D Systems); anti-LILRB1 clone 3D3-1D12 (Sigma-Aldrich); anti-LILRB1 clone GH1/75 (Novus Biologicals); and anti-LILRB4 antibodies that also cross-react with LILRB1, as described in US2018/0086829 (WO2016144728, University of Texas, see below). These antibodies are from non-human species.

Anti-LILRB2 antibodies disclosed include clone MAB2078 (R&D Systems); anti-LILRB2 clone 1D4 (Sigma-Aldrich); and anti-LILRB4 antibodies that also cross-react with LILRB1, as described in US2018/0086829 (WO2016144728, University of Texas, see below). These antibodies are from non-human species.

WO2020023268 also describes generation of anti-LILRB1 antibodies by immunization of XENOMOUSE® transgenic mice. Hybridoma supernatants with binding to human LILRB1 but no binding to human LILRA1 and human LILRA2 were selected and sequences of three exemplary anti-LILRB1 antibodies, 3C1, 30A10 and 19D6, are disclosed. The LILRB1-binding domains were determined for antibodies 3C1 (LILRB1 Domain 4), and 19D6 (LILRB1 Domain 4) and 30A10 (LILRB1 Domain 3).

TABLE 1
Summary of published data on binding specificity and properties of antibodies
disclosed in WO2020023268 (Y = Yes, N = No).

| | LILRB1 | LILRB2 | LILRA3 | LILRB3 | LILRB4 | LILRB5 | LILRA1 |
|---|---|---|---|---|---|---|---|
| | binding | binding | binding | binding | binding | binding | binding |
| MAB20171 | Y | N | N | | | N | N |
| MAB20172 | Y | N | | N | N | N | N |
| 3D3-1D12 | Y | | | | | | |
| GH1/75 | Y | | | | | | |
| MAB2078 | N | Y | N | | N | N | N |
| anti-LILRB2 1D4 | | Y | | | | | |
| (Sigma-Aldrich) | | | | | | | |
| Non-human anti- | Y | | | | Y | | |
| LILRB4 & LILRB1 | | | | | | | |
| cross reactive | | | | | | | |
| antibodies of | | | | | | | |
| US20180086829 | | | | | | | |
| (WO2016144728, | | | | | | | |
| below) | | | | | | | |
| 3C1 | Y | N | | | | | N |
| 19D6 | Y | N | | | | | N |
| 30A10 | Y | N | | | | | N |

| | | | | | LILRB1 | |
|---|---|---|---|---|---|---|
| | LILRA2 | LILRA4 | LILRA5 | LILRA6 | to HLA-G | LILRB1 |
| | binding | binding | binding | binding | Blocker | Domain |
| MAB20171 | | | | | | |
| MAB20172 | N | | | | | |
| 3D3-1D12 | | | | | | |
| GH1/75 | | | | | | |
| MAB2078 | N | N | N | | | |
| anti-LILRB2 1D4 | | | | | | |
| (Sigma-Aldrich) | | | | | | |
| Non-human anti- | | | | | | |
| LILRB4 & LILRB1 | | | | | | |
| cross reactive | | | | | | |
| antibodies of | | | | | | |
| US20180086829 | | | | | | |
| (WO2016144728, | | | | | | |
| below) | | | | | | |
| 3C1 | N | | | | | D4 |
| 19D6 | N | | | | | D4 |
| 30A10 | N | | | | | D3 |

WO2020136145 (Innate Pharma) describes LILRB1 antibodies that bind the D1 or D4 region of LILRB1. It is said that many of the anti-LILRB1 antibodies bound LILRA3 in addition to LILRB1, either alone (i.e. LILRB1 and LILRA3 cross-reactive) or with additional binding to LILRB2 or LILRB3. Antibodies 1C11, 1D6, 9G1, 19F10a, 27G10, and commercial antibodies 586326 and 292305 bound to LILRB1 and LILRA3. Antibody 586326 (mouse IgG2b, Bio-Techne #MAB30851), a murine monoclonal IgG2b antibody is the only antibody reported in WO2020136145 to bind to LILRB2 in addition to LILRB1 and LILRA3; however, no experimental data is shown to support this assertion and this contradicts the technical information for this commercially-available antibody, which discloses that the antibody was raised against Mouse myeloma cell line NS0-derived recombinant human LILRA1/CD85I/LIR-6 Pro17-Asn461 and it is stated that “In direct ELISAs, 100-400% cross-reactivity with recombinant human (rh) ILT2 is observed and no cross-reactivity rhILT3, 4, 5, 6, rhLIR-7 or -8 is observed”. Thus according to the supplier, Antibody 586326 shows 100-400% cross-reactivity with recombinant human LILRB1 and LILRA1 and no cross-reactivity with recombinant human LILRB2, LILRB3, LILRB4, LILRA3, LILRA4 or LILRA6 is observed. Commercial antibody 292305 bound LILRB3 in addition to LILRB1 and LILRA3. Commercial antibody 292319 bound to LILRA2 in addition to LILRB1. A subset of antibodies exemplified by 3H5, 12D12, 26D8, 18E1, 27C10 and 27H5 bound only to LILRB1 and no other LILR family member protein.

TABLE 2
Summary of published data on binding specificity and properties of antibodies
disclosed in WO2020136145 (Y = Yes, N = No).

| | LILRB1 | LILRB2 | LILRA3 | LILRB3 | LILRB4 | LILRB5 | LILRA1 | LILRA2 |
|---|---|---|---|---|---|---|---|---|
| | binding | binding | binding | binding | binding | binding | binding | binding |
| 1C11, 1D6, | Y | | Y | | | | | |
| 9G1, 19F10a, | | | | | | | | |
| 27G10 | | | | | | | | |
| 586326 | Y | [x] | [x] | | | | Y | |
| 292305 | Y | | Y | Y | | | | |
| 292319 | Y | | | | | | | Y |
| 3H5, 12D12, | Y | N | N | N | N | N | N | N |
| and 27H5 | | | | | | | | |
| 26D8, 18E1, | Y | N | N | N | N | N | N | N |
| 27C10 | | | | | | | | |
| 2H2B, 48F12, | Y | | | | | | | |
| 1E4C, 1A9, | | | | | | | | |
| 3F5 and 3A7A | | | | | | | | |

| | LILRA4 | LILRA5 | LILRA6 | LILRB1 to HLA -G | LILRB1 |
|---|---|---|---|---|---|
| | binding | binding | binding | Blocker | Domain |
| 1C11, 1D6, | | | | Y | |
| 9G1, 19F10a, | | | | | |
| 27G10 | | | | | |
| 586326 | | | | | |
| 292305 | | | | | |
| 292319 | | | | | |
| 3H5, 12D12, | N | N | N | Y | D1 |
| and 27H5 | | | | | |
| 26D8, 18E1, | N | N | N | Y | D4 |
| 27C10 | | | | | |
| 2H2B, 48F12, | | | | Y | D1 |
| 1E4C, 1A9, | | | | | |
| 3F5 and 3A7A | | | | | |

Antibodies 3H5, 12D12, 26D8, 18E1, 27C10, 27H5, 1C11, 1D6, 9G1, 19F10a and 27G10 all blocked LILRB1 binding to HLA-G and HLA-A2. Antibodies 12D12, 2A8A, 2A8B, 2A9, 2B11, 2C4, 2C8, 2D8, 2E2B, 2E2C, 2E8, 2E11 2G5, 2H2A, 2H2B, 2H12, 1A9, 1A10B, 1A10C, 1A10D, 1E4B, 1E4C, 3A7A, 3A7B, 3A8, 3B5, 3E5, 3E7A, 3E7B, 3E9A, 3E9B, 3F5, 4A8, 4C11B, 4E3A, 4E3B, 4H3, 5C5, 5D9, 6C6, 10H1, 48F12, 15D7, 2C3 all blocked LILRB1 (ILT2) binding to HLA-G and HLA-A2.

Antibodies 3H5, 12D12 and 27H5 bound an epitope in domain D1 of LILRB1. Antibodies 26D8, 18E1 and 27C10 all bound to the D4 domain of LILRB1. Antibodies 12D12, 2H2B, 48F12, 1E4C, 1A9, 3F5 and 3A7A bound an epitope in domain D1 of LILRB1. Antibodies 26D8 and 18E1 lost binding following amino acid substitutions F2991, Y300R, D301A, W328G, Q378A, K381N or substitutions W328G, Q330H, R347A, T349A, Y350S, Y355A. 26D8 furthermore lost binding to mutant LILRB1 with amino acid substitutions D341A, D342S, W344L, R345A, R347A, while antibody 18E1 had a decrease in binding (but not complete loss of binding) to the same mutant. 27C10 also lost binding to the same mutant, but not to any other mutant. It is suggested that these amino acid residues, together with lack of binding to human LILRA3 polypeptide, can identify an epitope that characterizes anti-LILRB1 antibodies that enhance cytotoxicity in primary NK cells.

The disclosed LILRB1 antibodies were characterized by their ability to block the interactions between HLA-G or HLA-A2 expressed at the surface of cell lines and recombinant LILRB1 protein was assessed by flow cytometry. This enabled the identification of a panel of anti-LILRB1 antibodies that were highly effective in blocking the interaction of LILRB1 with its HLA class I ligand HLA-G. Antibodies 3H5, 12D12, 26D8, 18E1, 27C10, 27H5, 1C11, 1D6, 9G1, 19F10a and 27G10 all blocked LILRB1 binding to HLA-G and HLA-A2. Such blocking antibodies are suggested to be useful in the treatment of a wide range of cancers characterized by tumour cells that express HLA-G (and/or other LILRB1 ligands such as HLA-A2) or HLA-E in addition to HLA-G. The neutralization of binding of LILRB1 to HLA is therefore considered a desirable antibody feature.

LILRA3 is naturally present as a soluble protein and binds HLA class I molecules. It is suggested that LILRA3 may thereby compete with LILRB1 for HLA class I molecule binding, acting as an inhibitor of LILRB1 signaling; consequently, Identification of antibodies that bind LILRB1 without binding to LILRA3 is considered desirable.

GHI/75 is a mouse monoclonal LILRB1 antibody that has been shown to increase macrophage phagocytotic activity by enhancing anti-CD47-blockade-mediated cancer cell phagocytosis, it has not been demonstrated to have an effect on its own (see Barkal et al., “Engagement of MHC class I by the inhibitory receptor LILRB1 suppresses macrophages and is a target of cancer immunotherapy”, Nat. Immunol. January; 19 (I): 76-84).

WO2021028921 (Biond Biologics) describes antibodies 19E3, 15G8 and 17F2 that bind LILRB1. Cross-reactivity to LILRA3 and LILRA1 was examined using binding ELISA; none of the antibodies cross-reacted with human LILRA3 or LILRA1. 15G8 binds to an epitope in the interdomain between D1 and D2, believed to be the interaction region of LILRB1 that binds with beta-2-microglobulin (B2M) when complexed with HLA.

Antibodies were selected according to their preferable binding, cross-reactivity profile and functional activity in the various assays examined. Each LILRB1 antibody was shown to block LILRB1-biotin binding to cells expressing HLA-G, and the 15G8 antibody blocked the LILRB1 MHC-I interaction. Functional blocking was examined in human Jurkat cells (T cells) by co-culturing Jurkat cells expressing LILRB1 with or without A375 cancer cells that express MHC-I. It is shown that the MHC-I from the cancer cells strongly inhibited secretion of the pro-inflammatory cytokine IL-2 and that 15G8 antibody, which blocks the LILRB1/MHC-I interaction, increased IL-2 secretion in a dose dependent manner. The inhibitory effect of LILRB1 was enhanced by transfecting the A375 cancer cells with HLA-G, making them MHC-I and HLA-G positive.

It is also shown that the blocking LILRB1 antibodies 19E3, 15G8 and 17F2 can also enhance the phagocytosis of HLA-G positive A375 cells by macrophages, and that the LILRB1 blocking antibody 15G8 can enhance the phagocytosis of various MHC-I positive cancer cell lines. The presence of a LILRB1 blocking antibody during the differentiation of macrophages from monocytes was shown to increase the expression of HLA-DR and CD80, markers of inflammatory macrophage phenotype.

TABLE 3
Summary of published data on binding specificity and properties of antibodies
disclosed in WO2021028921 (Y = Yes, N = No).

| | LILRB1 | LILRB2 | LILRA3 | LILRB3 | LILRB4 | LILRB5 | LILRA1 | LILRA2 |
|---|---|---|---|---|---|---|---|---|
| | binding | binding | binding | binding | binding | binding | binding | binding |
| 19E3 | Y | | N | | | | N | |
| 15G8 | Y | | N | | | | N | |
| 17F2 | Y | | N | | | | N | |

| | LILRA4 | LILRA5 | LILRA6 | LILRB1 to HLA-G | LILRB1 |
|---|---|---|---|---|---|
| | binding | binding | binding | Blocker | Domain |
| 19E3 | | | | Y | |
| 15G8 | | | | Y | D1-D2 |
| | | | | | interdomain |
| 17F2 | | | | Y | |

WO2022034524 (Biond Biologics) describes antibodies that bind an epitope within the ILT2 (LILRB1) interdomain between the D1 and D2 domains, the interaction domain between ILT2 and beta-2-microglobulin (B2M), that directly block the interaction between LILRB1 and its HLA-G ligand. An anti-ILT2 antibody used as a monotherapy was shown to enhance phagocytosis of cancer cells.

WO2022026360 (University of Texas) describes antibodies that bind LILRB1 at D1-D2, in particular at an epitope located within the linker region between the D1 and D2 domain of human LILRB1 and that block the interaction of LILRB1 with HLA-G. Antibodies are disclosed that bind LILRB1, or to LILRB1 and LILRA1, with no binding to the other LILRB or LILRA family members.

WO2022025585 (LG Chem) describes antibodies specific for LILRB1. Antibodies 10, 11, and 13 increased cell death of HLA-G overexpressed HEK293 cells by the natural killer cell KHYG-1 compared to human IgG4 Isotype control, indicating that these antibodies increase cytotoxicity of NK cells.

WO2021222544 (NGM) describes antibodies that bind to human LILRB1, human LILRB2, and both human LILRB1 and human LILRB2 including 73D1 and Hz73DI.vl, anti-LILRB1/anti-LILRB2 dual antagonist monoclonal antibodies. In addition to binding to LILRB1 and LILRB2, anti-LILRB1/LILRB2 antibodies show cross-reactivity with LILRA1, but not with LILRB3, LILRB4, LILRB5, LILRA2, LILRA4, LILRA5, and LILRA6. As part of the characterization process, the ability of exemplary antibodies to inhibit or block the interaction of LILRB1 or LILRB2 with their natural ligands was evaluated in competition experiments using a Biacore system. The natural ligands of LILRB1 and LILRB2 include, but are not limited to, HLA class I molecules, including HLA-A, HLA-B, HLA-C, HLA-E, and HLA-G. Anti-LILRB1 and anti-LILRB1/LILRB2 antibodies described therein inhibited the interactions between LILRB1 and its ligands. In addition, the anti-LILRB2 and anti-LILRB1/LILRB2 antibodies described inhibited the interactions between LILRB2 and its ligands. The anti-LILRB1/LILRB2 antibodies can bind to both targets, i.e., LILRB1 and LILRB2 and are also biologically functional in preventing the interactions of both targets with their ligands.

Phagocytosis assays were performed to further characterize the effect of anti-LILRB1, anti-LILRB2, and anti-LILRB1/LILRB2 antibodies on macrophage functions. Anti-LILRB1/LILRB2 antibodies (e.g., Hz73DI.vl) and anti-LILRB1 antibodies (e.g., 27F9) enhanced phagocytic activity of macrophages against Raji tumour cells opsonized with anti-CD47 antibody. Anti-LILRB2 antibodies (e.g., 48A5) had no effect on phagocytosis by macrophages. Antibody 24E7, an anti-LILRB1 antibody that does not disrupt MHC-I interaction, was unable to induce macrophage phagocytosis. These data suggest that the anti-LILRB1 and anti-LILRB1/LILRB2 antibodies that enhance macrophage phagocytosis do so by disrupting macrophage LILRB1 interaction with MHC-I on tumour cells, thereby inhibiting LILRB1-induced suppression of macrophages, and thus increasing macrophage phagocytosis of tumours. The anti-LILRB1 antibodies that are unable to block interaction with MHC-I, such as 24E7, do not induce macrophage phagocytosis.

Anti-LILRB1/LILRB2 antibodies as well as anti-LILRB1 and anti-LILRB2 antibodies were evaluated for their ability to cause PBMC pro-inflammatory cytokine release following LPS stimulation. LILRB2 and LILRB1/2 antibodies, but not a LILRB1-selective antibody, were able to induce an increase in release of pro-inflammatory TNF-alpha and GM-SCF following LPS stimulation. These data show that LILRB2 can suppress pro-inflammatory cytokine release from PBMCs following LPS stimulation. LILRB1/2 antibodies and LILRB2 antibodies, but not a LILRB1 selective antibody, were able to reduce the immunosuppressive activity of MDSC in an MLR assay. MLR assays are used to determine allogeneic T cell activation. Macrophages are traditionally characterized as either pro-inflammatory (MI) or immune suppressive (M2) based on surface expression markers CD80, CD86 (MI), CD163, CD204, and CD206 (M2). Hz73DI.vl (dual LILRB1/LILRB2 antibody) induced a decrease in M2-like macrophage phenotypic markers CD163, CD204, and CD206 and additional M2-like markers CD14 and CD209, consistent with an M2-like to M1-like polarization of the monocytes during differentiation. Anti-LILRB2 specific antibody 48A5, but not anti-LILRB1 specific antibody 27F9, induced a comparable change in the M1 and M2-like marker profile as the dual LILRB1/LILRB2 antibody suggesting that the LILRB2 interaction is responsible for the M2- to M1-like polarization.

These data indicate that anti-LILRB1/LILRB2 antibodies increase macrophage phagocytosis in the presence of CD47 antibody via LILRB1 and induce a more pro-inflammatory M1-like phenotype during macrophage differentiation via LILRB2, mediated by inhibition of LILRB1 or LILRB2 interaction with MHC-1.

TABLE 4
Summary of published data on binding specificity and properties of antibodies
disclosed in WO2021222544 (Y = Yes, N = No).

| | LILRB1 | LILRB2 | LILRA3 | LILRB3 | LILRB4 | LILRB5 | LILRA1 | LILRA2 |
|---|---|---|---|---|---|---|---|---|
| | binding | binding | binding | binding | binding | binding | binding | binding |
| 27F9 | Y | N | | N | N | N | Y | N |
| 47C8, | N | Y | | N | N | N | Y | N |
| 48A5, | N | Y | | N | N | N | Y | N |
| 47H6, | Y | Y | | N | N | N | Y | N |
| 51A1 | Y | Y | | N | N | N | Y | N |
| 64A12 | Y | Y | | N | N | N | Y | N |
| 73C4 | Y | Y | | N | N | N | Y | N |
| 73D1 | Y | Y | | N | N | N | Y | N |
| Hz73DI.vl | Y | Y | | N | N | N | Y | N |

| | LILRA4 | LILRA5 | LILRA6 | LILRB1 to HLA -G | LILRB1 |
|---|---|---|---|---|---|
| | binding | binding | binding | Blocker | Domain |
| 27F9 | N | N | N | Y | |
| 47C8, | N | N | N | Y | |
| 48A5, | N | N | N | Y | |
| 47H6, | N | N | N | Y | |
| 51A1 | N | N | N | Y | |
| 64A12 | N | N | N | Y | |
| 73C4 | N | N | N | Y | |
| 73D1 | N | N | N | Y | |
| Hz73DI.vl | N | N | N | Y | |

WO2018187518 (Merck, Agenus) disclosed the anti-LILRB2 (anti-ILT4) antibody 1E1 that bound a non-linear conformational epitope that overlaps with an epitope bound by HLA-G. Epitope characterization was provided only for 1E1. Other anti-LILRB2 antibodies 1G2, 2A6, 2D5, 3E6, 3G7, 2C1 and 5A6 were disclosed, with specific characteristics such as the ability to bind cynomolgus ILT4 (LILRB2), to block HLA-G Fc ligand binding to ILT4, rescue of spontaneous IL2 suppression and of HLA-G-dependent suppression.

WO2019126514 (Jounce) discloses anti-LILRB2-specific antibodies, none of the antibodies disclosed bind LILRB1, LILRB4, LILRB5, LILRA3, and LILRA6. WO2019126514 disclosed anti-LILRB2 antibodies able to block the interaction of HLA-G/A and LILRB2. A positive correlation between M1-promoting activity (as measured by TNFalpha increase) and the ability for anti-LILRB2 mAbs to block HLA-G/A: LILRB2 interactions was reported. Chimeric (hIgG4) anti-LILRB2 antibodies were selected based on specificity to cell-expressed hLILRB2 over the ten other human LILR family members, ability to block the ligand interactions to cell-expressed LILRB2, and ability to convert M2-like macrophages to M1-like macrophages having an inflammatory activation status in a primary human macrophage assay. Select LILRB2-specific, ligand blocking antibodies were additionally screened for binding to non-human primate (NHP) monocytes. JTX-8064 (Jounce) is a humanized IgG4 monoclonal antagonist antibody that selectively binds LILRB2, thereby preventing LILRB2 from binding its ligands, classical and non-classical MHC I molecules. By blocking the ability of LILRB2 to bind HLA-A/B and/or HLA-G, a marker of immunotolerance on cancer cells, JTX-8064 was shown to enhance pro-inflammatory cytokine production in macrophages. Antagonism of LILRB2 has been reported to result in repolarization of human macrophages from an M2 (suppressive) to M1 (pro-inflammatory) phenotype, and enhancement of anti-tumour immunity in a mouse model.

WO 2016144728 A2 (Univ. Texas) identifies a group of antibodies that bind LILRB2, 3, and 4. FIG. 19 of that application shows the cross-reactivity of the antibodies against LILRB1-5, none of the antibodies bound LILRB1.

WO2022087188 (ImmuneOnc) describes antibody B2-19 antibody and its variants which bind specifically to LILRB2 and block the interaction of LILRB2 with multiple ligands that are involved in cancer-associated immune suppression including HLA-G, ANGPTLs, SEMA4A, and CD1d.

WO2022079045 describes antagonist antibodies that bind to human and macaque LILRB1 and/or LILRB2. None of the antibodies (B.1.2.1 and B.1.2.2) bound with any of the human LILRA2, LILRA4, LILRA5, LILRB3, LILRB4 nor LILRB5, nor with any of the macaque LILRA1.1, LILRA1.2, LILRA2.1, LILRA2.2, LILRA4, LILRB3 nor LILRB4.

WO2019144052 (Adanate) discusses antibodies that bind to various LILRB and LILRs, however it provides no antibody sequences, Antibodies 5G11.H6, 9C9.E6, 9C9.D3, 5G11.G8 and 16D11.D10 are said to bind LILRB1, LILRB2, LILRB3, LILRB5, LILRA1, LILRA3 and LILRA5, but do not bind LILRB4, LILRA2, and LILRA4, and they are HLA-G blocking antibodies.

Through cis or trans interactions with human leukocyte antigen (HLA)-G, the two most abundantly expressed inhibitory LILRs, LILRB1 and LILRB2 (LILRB1/2, also known as CD85j/d and ILT2/4), are involved in immunotolerance in pregnancy and transplantation, autoimmune diseases, and immune evasion by tumours. LILRB1/2 contain four extracellular Ig-like domains, D1, D2, D3, and D4. D1D2 is thought to be responsible for binding to HLA class I (HLA-I), however, the roles of D3D4 are unclear. Crystallography of the complex structure of four-domain LILRB1 and HLA-G1 supports the model that D1D2 is responsible for HLA binding, while D3D4 acts as a scaffold. (Wang et al. (2020) Cell Mol Immunol. 2020 September; 17 (9): 966-975. doi: 10.1038/s41423-019-0258-5. Epub 2019 Jul. 4).

LILRB1 and LILRB2, and in particular the D1D2 domains responsible for ligand binding, represent an attractive target for anti-cancer approaches where immune regulatory processes have been subverted to evade anti-tumour immunity, by inducing macrophage phagocytosis by blocking LILRB1 interaction and induction of pro-inflammatory macrophage reprogramming by blocking LILRB2 ligand interaction, including MHC-1 and HLA-G. However, pre-clinical efficacy in models of cancer following LILRB1 antibody or LILRB2 antibody treatment have so far been mixed, with partial growth inhibition reported or tumour regression only being observed in a subset of animals. There is therefore a need to develop further approaches to targeting the LILR family.

### STATEMENTS OF INVENTION

The invention provides:

1. An antigen-binding protein capable of binding specifically to human LILRB1 and to human LILRB2, wherein the antigen-binding protein does not block the interaction of human LILRB1 with HLA-G tetramer and/or the antigen-binding protein does not block the interaction of human LILRB2 with HLA-G tetramer, and the antigen-binding protein is capable of reprogramming macrophage.

2. An antigen-binding protein of clause 1 that is capable of reprogramming fully differentiated macrophage to an anti-tumoural (pro-inflammatory) phenotype.

3. An antigen-binding protein, of clause 1 or clause 2, wherein reprogramming is indicated/detected by induction of a marker of macrophage reprogramming.

4. An antigen-binding protein, of any preceding clause, wherein reprogramming is indicated/detected by release of a pro-inflammatory cytokine from the macrophage following exposure of the macrophage to the antigen-binding protein and a stimulus selected from LPS stimulation, stimulation with R848, IL1beta, HMGB1 peptide, c-di-AMP and poly(I:C).

5. An antigen-binding protein, of any preceding clause, wherein reprogramming is indicated/detected by release of a pro-inflammatory cytokine TNF alpha and/or GM-CSF from macrophages following exposure to antigen-binding protein and LPS stimulation.

6. An antigen-binding protein, of any preceding clause, wherein the antigen-binding protein has one or more property selected from:
(a) stimulates the production of GM-CSF and/or TNFalpha on LPS stimulation of iPS-derived macrophage and/or primary-monocyte-derived macrophage,
(b) stimulates the production of GM-CSF and/or TNFalpha on LPS stimulation of iPS-derived macrophage expressing LILRB1,
(c) stimulates the production of GM-CSF and/or TNFalpha on LPS stimulation of primary-monocyte-derived macrophage expressing LILRB1 and LILRB2,
(d) stimulates the production of GM-CSF and/or TNFalpha on LPS stimulation of human macrophages expressing LILRB1 and LILRB2, and,
(e) stimulates the production of GM-CSF and/or TNFalpha on LPS stimulation of human macrophages expressing LILRB1.

7. An antigen-binding protein of any preceding clause, wherein the antigen-binding protein has one or more property selected from the ability to:
(a) induce phagocytosis,
(b) induce phagocytosis in the absence of a second signal,
(c) induce phagocytosis in the absence of a second antibody (e.g., an anti-CD47 antibody or an anti-EGFR antibody),
(d) induce phagocytosis in the absence of second, opsonizing antibody (e.g., an anti-CD47 antibody or an anti-EGFR antibody),
(e) induce phagocytosis of cancer cells in the absence of second, opsonizing antibody (e.g., a tumour binding antibody), and,
(f) induce phagocytosis of MHC Class I positive and/or MHC Class I negative cancer cells.

8. An antigen-binding protein of any preceding clause, capable of binding specifically to:
(a) human LILRB1, human LILRB2 and human LILRA3;
(b) human LILRB1, human LILRB2, human LILRA3 and human LILRA1;
(c) human LILRB1, human LILRB2, human LILRB3, human LILRA3, human LILRA4, and human LILRA6; and/or,
(d) human LILRB1, human LILRB2, human LILRB3, human LILRA1, human LILRA3, human LILRA4, and human LILRA6.

9. An antigen-binding protein of any preceding clause that does not bind to human LILRB4, human LILRB5, human LILRA2 or human LILRA5.

10. An antigen-binding protein of any preceding clause wherein binding to is assessed by flow cytometry or ELISA.

11. An antigen-binding protein of any preceding clause, capable of binding specifically to a homologue of LILRB1/2 ectodomain of rhesus monkey (SEQ ID NO: 43) and/or cynomolgus monkey (SEQ ID NO: 44).

12. An antigen-binding protein according to any one of the preceding clauses that binds an epitope common to:
(a) human LILRB1, human LILRB2 and human LILRA3;
(b) human LILRB1, human LILRB2, human LILRA3 and human LILRA1;
(c) human LILRB1, human LILRB2, human LILRB3, human LILRA3, human LILRA4, and human LILRA6; and/or,
(d) human LILRB1, human LILRB2, human LILRB3, human LILRA1, human LILRA3, human LILRA4, and human LILRA6.

13. An antigen-binding protein according to any one of the preceding clauses wherein the epitope is formed by:
(a) the sequence AEFPMGPVTSAHAGT (SEQ ID NO: 78) of human LILRB1;
(b) the sequence AEFPMGPVTSAHAGT (SEQ ID NO: 78) and the sequence LTHPSDPLEL (SEQ ID NO: SEQ ID NO: 79) of human LILRB1;
wherein the epitope is mapped using hydrogen-deuterium exchange (HDX) mass spectrometry method.

14. An antigen-binding protein according to any one of the preceding clauses wherein the epitope is formed by:
(a) sequence FVLYKDGERDF (SEQ ID NO: 80) in human LILRB1, sequence GYDRFVLYKEGERD (SEQ ID NO: 81) in human LILRB2, and sequence YDRFVLYKEWGRD (SEQ ID NO: 82) in human LILRA3;
(b) sequence SSEWSAPSDPLD (SEQ ID NO: 83) in LILRB1, sequence ECSAPSDPLDI (SEQ ID NO: 84) in LILRB2, and sequence SEWSAPSDPLD (SEQ ID NO: 85) in LILRA3;
(c) sequence LQCVSDVGYD (SEQ ID NO: 86) in LILRB2 and sequence FQCGSDAGYDRF (SEQ ID NO: 87) in LILRA3;
(d) sequence FLLTKEGAADDPW (SEQ ID NO: 88) in LILRB1 and sequence AADAPLRLRSIHEY (SEQ ID NO: 89) in LILRB2;
(e) sequence RSYGGQYR (SEQ ID NO: 90) in LILRB1 and sequence PVSRSYGGQYRC (SEQ ID NO: 91) in LILRB2;
(f) sequence LDILIAGQFYD (SEQ ID NO: 92) in LILRB1, sequence APSDPLDILI (SEQ ID NO: 93) in LILRB2, and sequence PSDPLDILI (SEQ ID NO: 94) in LILRA3;
wherein the epitope is mapped using binding to peptide microarrays.

15. An antigen-binding protein according to any one of the preceding clauses, wherein the antigen-binding protein is an antibody or an antigen-binding fragment thereof.

16. An antigen-binding protein according to any one of the preceding clauses, wherein the antigen-binding protein is a human antibody or an antigen-binding fragment thereof.

17. An antigen-binding protein according to any one of the preceding clauses, wherein the antigen-binding protein is a monoclonal antibody, such as a human monoclonal antibody.

18. An antigen-binding protein according to any one of the preceding clauses, wherein the antigen-binding protein comprises an Fc, such as a human IgG1 Fc or human IgG4 Fc.

19. An antigen-binding protein according to any one of the preceding clauses, comprising the six CDRs (HCDR1, HCRD2, HCDR3, LCDR1, LCDR2 and LCDR3, respectively) of an antibody selected from:
(a) Antibody 1 of SEQ ID NO: 1, SEQ ID NO: 2, SEQ ID NO: 3, SEQ ID NO: 4, SEQ ID NO: 5 and SEQ ID NO: 6;
(b) Antibody 2 of SEQ ID NO: 9, SEQ ID NO: 10, SEQ ID NO: 11, SEQ ID NO: 12, SEQ ID NO: 13 and SEQ ID NO: 14;
(c) Antibody 3 of SEQ ID NO: 17, SEQ ID NO: 18, SEQ ID NO: 19, SEQ ID NO: 20, SEQ ID NO: 21 and SEQ ID NO: 22;
(d) Antibody 4 of SEQ ID NO: 25, SEQ ID NO: 26, SEQ ID NO: 27, SEQ ID NO: 28, SEQ ID NO: 29 and SEQ ID NO: 30; and
(e) Antibody 5 of SEQ ID NO: 33, SEQ ID NO: 34, SEQ ID NO: 35, SEQ ID NO: 36, SEQ ID NO: 37 and SEQ ID NO: 38;
wherein the sequences are defined using Kabat nomenclature.

20. An antigen-binding protein according to any one of the preceding clauses, comprising a VH and VL, respectively, of an antibody selected from:
(a) Antibody 1 of SEQ ID NO: 7 and SEQ ID NO: 8;
(b) Antibody 2 of SEQ ID NO: 15 and SEQ ID NO: 16;
(c) Antibody 3 of SEQ ID NO: 23 and SEQ ID NO: 24;
(d) Antibody 4 of SEQ ID NO: 31 and SEQ ID NO: 32; and
(e) Antibody 5 of SEQ ID NO: 39 and SEQ ID NO: 40;
wherein the sequences are defined using Kabat nomenclature.

21. An antigen-binding protein, such as a human antibody or an antigen-binding fragment thereof, that is capable of competing for binding to human LILRB1, human LILRB2 and/or human LILRA3 with an antigen-binding protein, such as an antibody or an antigen-binding fragment thereof, according to any one of the preceding clauses.

22. An antigen-binding protein, such as a human antibody or an antigen-binding fragment thereof, according to clause 21, wherein competition for binding is assessed using a competition assay selected from a cell-based binding assay, a cell-free binding assay, an immunoassay, ELISA, HTRF, flow cytometry, fluorescent microvolume assay technology (FMAT) assay, Mirrorball, high content imaging based fluorescent immunoassays, radioligand binding assays, bio-layer interferometry (BLI), surface plasmon resonance (SPR) and thermal shift assays.

23. A composition comprising an antigen-binding protein according to any one of clauses 1 to 22 and a diluent.

24. An antigen-binding protein according to any one of clauses 1 to 22 or a composition according to clause 23:
(a) for use as a medicament;
(b for use as a medicament for the treatment of cancer;
(c) for use in the treatment of a cancer;
(d) for use in the manufacture of a medicament for the treatment of a cancer;
wherein optionally the cancer of (b), (c) or (d) is selected from:
   (i) Acute Myeloid Leukemia (AML), Bladder Urothelial Carcinoma (BLCA), Brain Lower Grade Glioma (LGG), Breast invasive carcinoma (BRCA), Esophageal carcinoma (ESCA), Glioblastoma multiforme (GBM), Head and Neck squamous cell carcinoma (HNSC), Kidney renal clear cell carcinoma (KIRC), Kidney renal papillary cell carcinoma (KIRP), Liver hepatocellular carcinoma (LIHC), Lung adenocarcinoma (LUAD), Lung squamous cell carcinoma (LUSC), Pancreatic adenocarcinoma (PAAD), Sarcoma (SARC), Skin Cutaneous Melanoma (SKCM), Stomach adenocarcinoma (STAD), Testicular Germ Cell Tumours (TGCT), Thymoma (THYM), Thyroid carcinoma (THCA), Uterine Carcinosarcoma (UCS), Uterine Corpus Endometrial Carcinoma (UCEC), Uveal Melanoma (UVM), colorectal cancer, prostate cancer, pediatric cancers, lymphomas and leukemias such as DLBCL, NHL, multiple myeloma, Hodgkin lymphoma;
   (ii) cancer positive for LILRB1 or LILRB2 or both LILRB1 and LILRB2;
   (iii) cancer positive for immunosuppressive macrophages (as measured by CD163 or CD68 positivity) and/or tumour infiltrating T cells;
   (iv) cancer with increased or decreased expression of classical or non-classical MHC Class I;
   (v) cancer positive from one or more of LILRB1, LILRB2, LILRB3, LILRA1, LILRA3, LILRA4, and LILRA6;
   (e) for use as a medicament for the treatment of an immunosuppressive disease;
   (f) for use in the treatment of an immunosuppressive disease; or
   (g) for use in the manufacture of a medicament for the treatment of an immunosuppressive disease.

25. A method of treatment of a cancer, or of treatment of an immunosuppressive disease, comprising administration of an antigen-binding protein of any one of clauses 1 to 22, or a composition according to clause 23, to a subject.

26. An isolated recombinant DNA or RNA sequence comprising a sequence encoding an antigen-binding protein of any one of clauses 1 to 22.

27. An isolated recombinant DNA sequence of clause 26 which is a vector, optionally wherein the vector is an expression vector.

28. An isolated recombinant DNA sequence of clause 26 or 27 encoding an antigen-binding protein of any one of clauses 1 to 22 under control of a promoter.

29. A host cell comprising a DNA or RNA sequence according to any one of clauses 26 to 28, optionally wherein the host cell is capable of expressing an antigen-binding protein of any one of clauses 1 to 22.

30. A method of making an isolated antigen-binding protein of any one of clauses 1 to 22 comprising culturing a host cell of clause 29 in conditions suitable for expression of the isolated antibody or antigen-binding fragment thereof.

31. A method of identifying an antigen-binding protein, of any one of claims 1 to 22 comprising:
(a) providing one or more antigen-binding protein capable of binding to:
   (i) human LILRB1, LILRB2 and/or LILRA3;
   (ii) human LILRB1, LILRB2 and LILRA3 protein;
   (iii) human LILRB1, LILRB2, LILRA1 and LILRA3 protein;
   (iv) human LILRB1, LILRB2, LILRB3, LILRA3, LILRA4, and LILRA6 protein; and/or,
   (v) human LILRB1, LILRB2, LILRB3, LILRA1, LILRA3, LILRA4, and LILRA6 protein;
and performing one or more assessment selected from (b), (c) and (d):
(b) assessing the ability of the one or more antigen-binding protein to modulate one or more biological activity/phenotype of a human macrophage, such as to promote phagocytosis and/or pro-inflammatory cytokine release (such as TNFalpha or GM-SCF), expression of macrophage activation markers (such as HLA-DR and/or CD80) or reduction in expression of CD163 (a marker of macrophage activation to M1 phenotype);
(c) assessing the ability of the one or more antigen-binding protein to block binding of LILRB1 and/or LILRB2 to cells expressing a ligand of LILRB1 and/or LILRB2, e.g., HLA-G, and selecting one or more antibodies that bind to LILRB1, LILRB2 and LILRA3 and do not block binding of LILRB1 and/or LILRB2 to target cells expressing a ligand of LILRB1 and/or LILRB2, for example HLA-G;
(d) assessing the ability of the one more antigen-binding protein to block binding of ligand (e.g. HLA-G) to cells expressing LILRB1 and/or LILRB2, and selecting one or more antibodies that do not block binding of a ligand of LILRB1 and/or LILRB2, for example HLA-G, to a cell expressing LILRB1 and/or LILRB2; and
(e) selecting one or more antigen-binding protein capable of binding specifically to human LILRB1 and to human LILRB2, wherein the antigen-binding protein does not block the interaction of human LILRB1 with HLA-G tetramer and is capable of reprogramming macrophage, and, optionally,
(f) formulating the one or more antigen-binding protein into a composition with one or more excipient.

32. A method of identifying an antibody or antigen-binding fragment thereof, of any one of clauses 1 to 22 comprising:
(a) providing one or more antibody or antigen-binding fragment thereof capable of binding to:
   (i) human LILRB1, LILRB2 and/or LILRA3 protein;
   (ii) human LILRB1, LILRB2 and LILRA3 protein;
   (iii) human LILRB1, LILRB2, LILRA1 and LILRA3 protein;
   (iv) human LILRB1, LILRB2, LILRB3, LILRA3, LILRA4, and LILRA6 protein; and/or,
   (v) human LILRB1, LILRB2, LILRB3, LILRA1, LILRA3, LILRA4, and LILRA6 protein;
and performing one or more assessment selected from (b), (c) and (d):
(b) assessing the ability of the one more antibody or antigen-binding fragment thereof to block binding of LILRB1 and/or LILRB2 to cells expressing a ligand of LILRB1 and/or LILRB2, e.g., HLA-G, and selecting one or more antibodies that bind to LILRB1, LILRB2 and LILRA3 and do not block binding of LILRB1 and/or LILRB2 to target cells expressing a ligand of LILRB1 and/or LILRB2, for example HLA-G;
(c) assessing the ability of the one more antibody or antigen-binding fragment thereof to block binding of ligand (e.g. HLA-G) to cells expressing LILRB1 and/or LILRB2, and selecting one or more antibodies that do not block binding of a ligand of LILRB1 and/or LILRB2, for example HLA-G, to a cell expressing LILRB1 and/or LILRB2;
(d) assessing the ability of the one or more antibody or antigen-binding fragment thereof to modulate one or more biological activity/phenotype of a human macrophage, e.g., to promote phagocytosis and/or pro-inflammatory cytokine release (such as TNFalpha or GM-SCF), or expression of macrophage activation markers (such as HLA-DR and/or CD80).

The present invention provides antigen-binding proteins such as antibodies or antigen binding proteins, e.g., human monoclonal antibodies, each of which bind specifically to human LILRB1 and to human LILRB2. Antibodies of the invention also bind to human LILRA3 (e.g., Antibody 1, 2, 3, 4, 5), in some embodiments antibodies of the invention bind to LILRA3 and human LILRA1 (e.g., Antibody 3). In some embodiments an antibody of the invention binds to human LILRB1, LILRB2, LILRB3, LILRA3, LILRA4, and LILRA6. In some embodiments an antibody of the invention binds to human LILRB1, LILRB2, LILRB3, LILRA1, LILRA3, LILRA4 and LILRA6. In preferred embodiments, Antibodies 1 to 5 of the invention do not bind to human LILRB4, human LILRB5, human LILRA2 and human LILRA5,

Antibodies of the invention are “non-blocking” in that they do not disrupt the interaction of LILRB1 and/or LILRB2 with HLA-G.

By “does not block” “no blocking activity” or “non-blocking” or “not blocking”, it is meant that in an assay described herein the assay signal is more than 10% of the signal observed for the isotype control. The Isotype control is 100% of signal, blocking is less than 10% of the signal observed for the isotype control, non-blocking is more than 10% of the signal observed for the isotype control. The percent of blocking can be determined by normalizing to a negative control IgG. The percentage of blocking can be calculated at various concentrations of test antibody. Antibodies of the invention block the binding of HLA-G tetramer to LILRB1; blocking can be detected and/or quantified by any suitable means known in the art or described herein. For example, blocking of the HLA-G tetramer-LILRB1 interaction can be detected and/or quantified using a tetramer blocking assay as described herein. The ability of antibodies to block binding of the receptor to its ligand can be assessed using HEK293 cells over-expressing human LIRB1 receptor and incubating the cells with human HLA-G PE-labelled tetramers, in the absence or presence of 500 nM test antibodies; cell bound HLA-G can be quantified by flow cytometry.

Antibodies of the invention are able to induce reprogramming of macrophage, such as fully differentiated macrophage, to an anti-tumoural, pro-inflammatory phenotype. A macrophage with an anti-tumoural phenotype secretes high levels of pro-inflammatory cytokines such as GM-CSF, TNFa, IL1, IL6 and IL12. A macrophage that is reprogrammed to be an anti-tumoural macrophage will have increased secretion of one or more pro-inflammatory cytokine (e.g., GM-CSF, TNFa, IL1, IL6, and/or IL12) relative to macrophage before reprogramming. Surface markers of an anti-tumoural phenotype include CD80 high and CD86 high, CD206 low, CD209 low and CD163 low. A fully differentiated macrophage is a macrophage with adhesive properties, which expresses differentiation markers such as CD163, CD206 and/or 25F9. Human macrophage include human iPS-derived macrophage, human monocyte-derived macrophage, human tumour-derived macrophage and human ascites-derived macrophage, human mono/macrophage cell lines (such as THP-1, mono/mac, U937), and TAMs.

In some embodiments, antibodies of the invention (e.g., Antibodies 1, 3, and 5) are able to induce phagocytosis of cancer cells. Cancer cells include immortalized and/or transformed cell lines, cell lines including and those with an oncogene resulting in uncontrolled proliferation, cells isolated from human tumours, cells isolated from human ascites, and/or cells isolated from patient-derived tumour xenograft models.

Antibodies of the invention are able to induce reprogramming, and in some instances also macrophage phagocytosis, irrespective of the HLA status of tumour cells and in the presence or absence of LILRB1/2 ligand expression on the tumour cells. Hence, the physical interaction between ligand expressing tumour cells and LILRB1 and/or LILRB2 tumour-associated macrophages (TAM) is not a prerequisite for antibodies of the invention to reprogram TAM. Furthermore, Antibodies 1, 3, and 5 of the invention that can induce phagocytosis, are able to do so in the absence of a second signal or antibody, e.g., an anti-CD47 antibody or an anti-EGFR antibody. Antibodies of the invention induce pro-inflammatory cytokine release (such as TNFalpha or GM-SCF) and/or expression of macrophage activation markers (such as HLA-DR and/or CD80) from fully differentiated macrophages (indicating macrophage “reprogramming” to an anti-tumour phenotype (pro-inflammatory phenotype)). Antibodies of the invention do not bind to the ITAM domain-containing LILRA2.

LILR family members have overlapping and distinct patterns of ligand binding and expression and can be either immune-stimulatory or immunosuppressive. Therefore, to maximally activate anti-tumour immunity, tailored approaches are required that minimize or avoid immuno-stimulatory LILR signaling (LILRA1, 2, 4, 5, 6), while inhibiting immuno-suppressive signaling (LILRB1, 2). It may be advantageous to target both LILRB1 and LILRB2 receptors that have common ligands and expression patterns, to overcome compensatory resistance mediated by target redundancy. LILRB1 and LILRB2 have high homology, bind common HLAs, are both expressed on myeloid cells including macrophages, and both have intracellular immunosuppressive ITIM domains. Therefore, without wishing to be bound by theory, it may be advantageous to target both LILRB1 and LILRB2, while avoiding binding to the ITAM-domain containing LILRA2 and LILRA5 receptors.

LILRA3 is considered an “off-target”, i.e., an undesirable target, in the art, with LILRB1 binding without LILRA3 binding reported to be associated with NK cell cytotoxicity and selectivity for LILRB1 over LILRA3 being reported as a positive feature when selecting preferred antibodies. Furthermore, it has been suggested that LILRA3 as a soluble factor may compete with LILRB1 and LILRB2 for ligand binding, and thereby act as a naturally-occurring competitor of LILRB1 and LILRB2 ligand binding.

However, the inventors hypothesized that LILRA3 binding may be advantageous based upon (i) the potential immunosuppressive role of LILRA3 in humans implicated in clinical manifestation of inflammatory conditions associated with genetic loss-of-function mutation of LILRA3, and the reduced affinity of LILRA3 for HLA-G and HLA-A compared to LILRB1 and LILRB2. The inventors further investigated the expression of LILRA3 in other tumour types using the TCGA database, which identified the over-expression of LILRA3 in multiple cancer types (FIG. 1).

Bioinformatic analysis of single cell RNA sequencing data-sets across multiple cancer types comparing LILRB1 and LILRB2 expression, and specifically in melanoma cancer patients, also revealed mixed expression of LILRB1 and LILRB2 in the tumour environment LILRB1-positive, LILRB2-positive, and both LILRB1-positive and LILRB2-positive macrophages were identified (FIG. 2 and FIG. 3).

Antibodies of the invention are capable of binding LILRB1 and LILRB2. Antibodies of the invention are capable of binding LILRB1, LILRB2 and LILRA3. Antibodies of the invention are capable of binding LILRB1, LILRB2, LILRA3 and LILRA1. Antibodies of the invention do not bind specifically to LILRA2.

LILRB1 and LILRB2 bind MHC class I, however, change in MHC class I expression is a known tumour immune evasion mechanism, reducing tumour antigen presentation and subsequent T cell activation. For example, down-regulation of classical MHC class I (for example, HLA-A) is found in approximately 1 in 3 melanoma patients and is associated with innate and acquired resistance to T cell checkpoint therapies. Numerous cancer types also up-regulate non-classical MHC class I such as HLA-G. Tumours are also highly heterogeneous and the immune infiltrate, including macrophages, NK cells, and T cells, are not distributed evenly within the tumour microenvironment and may not always be in direct contact with ligand-expressing tumour cells. However, successful activation of such immune cell types will cause cytokine, and chemokine release and contribute to anti-tumoural immunity within the tumour. LILRB1 expression in the tumour microenvironment has also been associated with poor clinical response to immune therapy, even when HLA-G is not present. It would therefore be a significant advantage if LILRB1/2 antibodies were able to activate the anti-tumoural features of macrophages (such as GM-SCF release and phagocytosis) in a manner that is not dependent the interaction with ligand. Despite this, to date, the ability of antibodies not only to bind to LILRB1 and/or LILRB2, but also to block ligand interaction has been a key part of antibody selection during drug discovery with binding and blocking MHC class I and HLA-G interaction taken as an important feature associated with effective induction of macrophage reprogramming and phagocytosis. Consistent with this strategy, non-ligand blocking LILRB1 antibodies have been used as controls in antibody characterization due to a lack of functional effect, as described above. Taken together this suggests a drug discovery strategy to identify functionally active, non-ligand blocking (hereafter “non-blocking”) LILRB1 and LILRB2 antibodies is unlikely to be successful. However, without wishing to be bound by theory, the inventors hypothesized that non-ligand blocking antibodies that can exert immune activation that is independent of the ligand expression status of the tumour, or proximity of the receptor-expressing immune cell to the ligand-expressing tumour cell, may increase patient therapeutic responses and the eligibility of patients for therapy.

In summary, the inventors conducted an antibody campaign against the extra-cellular domain of LILRB1, which is highly conserved to LILRB2 and has high sequence homology and structural conservation with the HLA-G binding region of LILRA3. Blocking and non-blocking antibodies were assessed in macrophage functional assays with the aim of identifying and comparing antibodies that are blocking and non-blocking for ligand binding. Although binding to LILRA3 is generally considered to be a disadvantage due to it being a natural inhibitor of LILRB1 and LILRB2 by competition for MHC-I binding, LILRA3 binders and non-binders were progressed. Phagocytosis and reprogramming assays were performed using MDMs and macrophages derived from induced pluripotent stem cells (iPSCs-DM). that expressed both LILRB1 and LILRB2.

39 antibodies were tested for the ability to reprogram fully differentiated macrophages and to induce phagocytosis. Of these 39 antibodies, only 5 antibodies were identified that were able to induce significant reprogramming of fully differentiated macrophages. Surprisingly, all 5 antibodies did not block HLA-G binding to LILRB1 or LILRB2 and they cross-competed (competed) with each other for LILRB1 binding in epitope binning and cross-competition ELISA experiments, whereas they did not compete with the other 34 antibodies or with Reference Antibody 2. These 5 antibodies also bound to LILRA3, but did not bind LILRA2. Antibodies 1 to 5 were found to bind to human LILRB1, LILRB2, LILRB3, LILRA1, LILRA3, LILRA4, and LILRA6 but not LILRB4, LILRB5, LILRA2, or LILRA5. Furthermore, these antibodies were able to induce macrophage phagocytosis of cancer cells positive and negative for MHC-I (including HLA-G) expression; therefore operating in a ligand independent manner, and were able to induce reprogramming of fully differentiated macrophages, as measured by cytokine release, of both iPSC-DM and MDMs to achieve the pro-inflammatory reprogramming of fully differentiated (mature) macrophages.

Antibodies of the invention demonstrate ligand-independent activity and can induce reprogramming and phagocytosis irrespective of the MHC-I status of the tumour, or proximity of macrophage to the tumour cell, which may translate into increased therapeutic response and patient benefit.

### DETAILED DESCRIPTION OF THE INVENTION

The invention relates to antigen-binding proteins and antigen-binding fragments thereof, such as antibodies and antigen-binding fragments thereof, particularly human antibodies and antigen-binding fragments thereof, capable of binding specifically to human LILRB1, human LILRB2, human LILRB3, human LILRA1, human LILRA3, human LILRA4 and human LILRA6.

Antibodies of the invention (e.g., Antibodies 1, 2, 3, 4, and 5) are capable of binding specifically to an epitope common to human LILRB1, human LILRB2 and human LILRA3. Antibodies of the invention (e.g., Antibodies 1, 2, 3, 4, and 5) do not bind specifically to human LILRA2. In some embodiments, an antibody of the invention (e.g., Antibody 3) is capable of binding specifically to an epitope common to human LILRB1, human LILRB2, human LILRA3 and human LILRA1. Antibodies 1 to 5 of the invention selectively bind to LILRB1, LILRB2, LILRB3, LILRA1, LILRA3, LILRA4 and LILRA6, but do not bind to the highly homologous LILRB4, LILRB5 and LILRA2.

In some embodiments, an antibody of the invention (e.g., Antibodies 1, 2, 3, 4, and 5) are capable of binding specifically to an epitope present in human LILRB1 ectodomain comprising domains D1, D2, D3, and D4 (SEQ ID NO: 41), but do not bind to a D1-D2 fragment of LILRB1 (SEQ ID NO: 42). The D1-D2 region is defined as amino acids 24 to 223 of human LILRB1, human LILRB2 and human LILRA3; the D3-D4 region is defined as amino acids 224 to 458 of human LILRB1 and LILRB2, and amino acids 224-439 in LILRA3 (FIG. 19). In some embodiments, an antibody of the invention (e.g., Antibodies 1, 2, 3, 4, and 5) are capable of binding specifically to an epitope formed by amino acid sequences in human LILRB1 ectodomains D3 and D4

Antibodies of the invention are cross-reactive in that they bind to homologues of LILRB1/2 ectodomain from rhesus monkey (SEQ ID NO: 43) and cynomolgus monkey (SEQ ID NO: 44).

Antibodies of the invention bind the major allelic variant forms of the human LILRB1 ectodomain with similar binding efficiency, “binding” denotes that the signal obtained for LILRB1 variant was at least more than 3 fold higher than that observed for the control protein.

Antibodies of the invention are capable of binding specifically to human LILRB1 and/or human LILRB2 expressed on a fully differentiated (mature) human macrophage, such as a TAM, and of modulating one or more biological activity/phenotype of the human macrophage selected from:
(a) promoting maintenance of an anti-tumoural phenotype, as assessed by increased release of TNFa and GM-CSF upon LPS stimulation and/or increased cancer cell phagocytosis,
(b) promoting a pro-inflammatory phenotype as assessed by increased release of TNFa and GM-CSF upon LPS stimulation,
(c) alleviating immunosuppression by production of pro-inflammatory cytokines as assessed by increased release of TNFa and GM-CSF upon LPS stimulation,
(d) promoting tumour-specific phagocytosis through a HLA-G independent mechanism as assessed by increased human macrophage phagocytosis of cancer cells that are negative or positive for HLA-G,
(e) promoting tumour-specific phagocytosis through a MHC Class I independent mechanism as assessed by increased human macrophage phagocytosis of cancer cells that are negative or positive for MHC Class I,
(f) promoting a pro-inflammatory macrophage phenotype as assessed by increased release of TNFa and GM-CSF upon LPS stimulation from human macrophages, but not from unstimulated peripheral blood mononuclear cells.

An antibody or antigen-binding fragment thereof of the invention may be produced by recombinant means.

A “recombinant antibody” is an antibody which has been produced by a recombinantly engineered host cell. An antibody or antigen-binding fragment thereof in accordance with the invention is optionally isolated or purified.

The term “antibody” or “antibody molecule” describes an immunoglobulin whether natural or partly or wholly synthetically produced. An antigen-binding protein of the invention may be an antibody, preferably a monoclonal antibody, and may be a human or non-human, chimeric or humanised.

The antibody molecule is preferably a monoclonal antibody, preferably a human monoclonal antibody. Examples of antibodies are the immunoglobulin isotypes, such as immunoglobulin G, and their isotypic subclasses, such as IgG1, IgG2, IgG3 and IgG4, as well as fragments thereof. The four human subclasses (IgG1, IgG2, IgG3 and IgG4) each contain a different heavy chain; but they are highly homologous and differ mainly in the hinge region and the extent to which they activate the host immune system. IgG1 and IgG4 contain 2 inter-chain disulphide bonds in the hinge region, IgG2 has 4 and IgG3 has 11 inter-chain disulphide bonds.

The terms “antibody” and “antibody molecule”, as used herein, includes antibody fragments, such as Fab and scFv fragments, provided that said fragments comprise a CDR-based antigen binding site for an epitope of the target antigen.

An antibody of the invention may be in monovalent or bivalent format and may or may not comprise an Fc. A bivalent antibody of the invention may be monoparatopic having two identical paratopes for epitope binding, or biparatopic having two different paratopes for epitope binding. A bivalent antibody of the invention may be a monospecific antibody that binds one epitope or may be a bispecific antibody that binds 2 different epitopes. A bivalent antibody of the invention may be a bispecific antibody that binds 2 different epitopes each from a different target antigen. A bivalent antibody of the invention may be a bispecific biparatopic antibody that binds two distinct epitopes (that are not over-lapping) on the same target antigen. For optimal macrophage re-programming activity, in particular under immunosuppressive M2 conditions, antibodies of the invention are preferably provided in a bivalent monoparatopic format and preferably comprise an Fc for engagement with Fc receptor on the macrophage membrane.

Examples of antibody fragments include but are not limited to Fv, Fab, Fab′, Fab′-SH, F(ab′)2; diabodies; linear antibodies; single-chain antibody molecules (e.g., scFv) and domain antibodies (sdAbs). Unless the context requires otherwise, the terms “antigen-binding protein”, “antibody” or “antibody molecule”, as used herein, is thus equivalent to “antibody or antigen-binding fragment thereof”.

Antibodies are immunoglobulins, which have the same basic structure consisting of two heavy and two light chains forming two Fab arms containing identical domains that are attached by a flexible hinge region to the stem of the antibody, the Fc domain, giving the classical ‘Y’ shape. The Fab domains consist of two variable and two constant domains, with a variable heavy (VH) and constant heavy 1 (CH1) domain on the heavy chain and a variable light (VL) and constant light (CL) domain on the light chain. The two variable domains (VH and VL) form the variable fragment (Fv), which provides the CDR-based antigen specificity of the antibody, with the constant domains (CH1 and VL) acting as a structural framework. Each variable domain contains three hypervariable loops, known as complementarity determining regions (CDRs). On each of the VH and VL the three CDRs (CDR1, CDR2, and CDR3) are flanked by four less-variable framework (FR) regions (FR1, FW2, FW3 and FW4) to give a structure FW1-CDR1-FW2-CDR2-FW3-CDR3-FW4. The CDRs provide a specific antigen recognition site on the surface of the antibody.

Both Kabat and ImMunoGeneTics (IMGT) numbering nomenclature may be used herein. Generally, unless otherwise indicated (explicitly or by context) amino acid residues are numbered herein according to the Kabat numbering scheme (Kabat et al., 1991, J Immunol 147 (5): 1709-19). For those instances when the IMGT numbering scheme is used, amino acid residues are numbered herein according to the ImMunoGeneTics (IMGT) numbering scheme described in Lefranc et al., 2005, Dev Comp Immunol 29 (3): 185-203.

Techniques for generation and isolation of exogenous, e.g., human, antibodies and fragments thereof, in transgenic non-human mammals, such as mice and rats, are well known in the art.

It is possible to take monoclonal and other antibodies and use techniques of recombinant DNA technology to produce other antibodies or chimeric molecules which generally retain the specificity of the original antibody. Such techniques may involve introducing the CDRs into a different immunoglobulin framework, or grafting variable regions onto a different immunoglobulin constant region. Introduction of the CDRs of one immunoglobulin into another immunoglobulin is described for example in EP-A-184187, GB2188638A or EP-A-239400. Alternatively, a hybridoma or other cell producing an antibody molecule may be subject to genetic mutation or other changes, which may or may not alter the binding specificity of antibodies produced.

Antibody humanisation involves the transfer, or “grafting”, of critical non-human amino acids onto a human antibody framework. Primarily this includes the grafting of amino acids in the complementarity-determining regions (CDRs), but potentially also other framework amino acids critical for the VH: VL interface and for orientation of the CDRs. Humanisation seeks to introduce human content to reduce the risk of immunogenicity, while retaining the original binding activity of the non-human parental antibody. The term “humanised antibody” is intended to refer to antibodies in which CDR sequences derived from the germline of another mammalian species have been grafted onto human framework sequences; optionally additional framework region modifications can be made within the human framework sequences. The term “antibody” includes antibodies in which CDR sequences derived from the germline of another mammalian species have been grafted onto human framework sequences and optimized (for example by affinity maturation), e.g., by modification or one more amino acid residues in one or more of the CDRs and/or in one or more framework sequence to modulate or improve a biological property of the antibody, e.g. to increase affinity, or to modulate the on rate and/or off rate for binding of the antibody to its target epitope. The term “humanised antibody” includes antibody that has been optimized (for example by affinity maturation), thus antibodies of the invention may be humanised, or both humanised and optimised, e.g., humanised and affinity matured.

As antibodies can be modified in a number of ways, the term “antigen-binding protein” or “antibody” should be construed as covering antibody fragments, derivatives, functional equivalents and homologues of antibodies, including any polypeptide comprising an immunoglobulin binding domain, an aptamer, affimer or bicyclic peptide, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023.

An example of an antibody fragment comprising both CDR sequences and CH3 domain is a minibody, which comprises a scFv joined to a CH3 domain (Hu et al. (1996) Cancer Res 56 (13): 3055-61).

A domain (single-domain) antibody is a peptide, usually about 110 amino acids long, comprising one variable domain (VH) of a heavy-chain antibody, or of an IgG. A single-domain antibody (sdAb), (e.g., nanobody), is an antibody fragment consisting of a single monomeric variable antibody domain. Like a whole antibody (comprising two heavy and two light chains), it is an antigen-binding protein able to bind selectively to a specific antigen. Domain antibodies have a molecular weight of only 12-15 kDa and are thus much smaller than antibodies composed of two heavy protein chains and two light chains (150-160 kDa), and domain antibodies are even smaller than Fab fragments (˜50 kDa, one light chain and half a heavy chain) and single-chain variable fragments (˜25 kDa, two variable domains, one from a light and one from a heavy chain). Single-domain antibodies have been engineered from heavy-chain antibodies found in camelids; these are termed VHH fragments. Cartilaginous fish also have heavy-chain antibodies (IgNAR, ‘immunoglobulin new antigen receptor’), from which single-domain antibodies called VNAR fragments can be obtained. A domain (single-domain) antibody may be a VH or VL. A domain antibody may be a VH or VL of human or murine origin. Although most single-domain antibodies are heavy chain variable domains, light chain single-domain antibodies (VL) have also been shown to bind specifically to target epitopes. Protein scaffolds have relatively defined three-dimensional structures and typically contain one or more regions which are amenable to specific or random amino acid sequence variation, to produce antigen-binding regions within the scaffold that are capable of binding to an antigen.

Binding in this context may refer to specific binding. The term “specific” may refer to the situation in which the antibody molecule will not show any significant binding to molecules other than its specific binding partner(s). The term “specific” is also applicable where the antibody molecule is specific for particular epitopes, as described herein that are carried by a number of antigens in which case the antibody molecule will be able to bind to the various antigens carrying the epitope.

An antigen-binding protein, such as an antibody or an antigen-binding fragment thereof of the invention binds to an epitope present in human LILRB1, human LILRB2 and human LILRA3. An antigen-binding protein, such as an antibody or an antigen-binding fragment thereof, binds to an epitope present in human LILRB1, human LILRB2 and human LILRA3, but does not bind to a LILRB1 ectodomain truncated protein fragments that contains only the D1 and D2 domains and not the D3 and D4 domains. An antigen-binding protein, such as an antibody or an antigen-binding fragment thereof binds to an epitope common to human LILRB1, human LILRB2 and human LILRA3. In some embodiments, an antigen-binding protein, such as an antibody or an antigen-binding fragment thereof binds to an epitope common to human LILRB1, human LILRB2, human LILRA3 and human LILRA1. In some embodiments an antibody of the invention binds to an epitope common to LILRB1, LILRB2, LILRB3, LILRA3, LILRA4, and LILRA6. In some embodiments an antibody of the invention binds to an epitope common to LILRB1, LILRB2, LILRB3, LILRA1, LILRA3, LILRA4 and LILRA6. In some embodiments an antibody of the invention binds to an epitope common to LILRB1, LILRB2, LILRB3, LILRA1, LILRA3, LILRA4, and LILRA6. Antibodies 1 to 5 of the invention selectively bind to LILRB1, LILRB2, LILRB3, LILRA1, LILRA3, LILRA4 and LILRA6, but do not bind to the highly homologous LILRB4, LILRB5, LILRA2 and LILRA5. An antibody of the invention is not an antibody listed any one of Tables 1 to 4 or otherwise described in the prior art of the background to the invention.

Putative epitopes on D3-D4 regions of human LILRB1, LILRB2, and LILRA3 molecules for Antibodies 1 to 5 were mapped by a CRO; PEPperPRINT; using their proprietary PEPperCHIP® linear and “conformational” peptide microarrays (FIG. 24). Strong binding to peptide microarrays was observed only for Antibody 2, suggesting it binds to relatively unstructured epitopes. Two putative epitopes present in all three target proteins corresponded to peptides: Epitope 1 (sequence FVLYKDGERDF (SEQ ID NO: 80) in LILRB1, sequence GYDRFVLYKEGERD (SEQ ID NO: 81) in LILRB2, and sequence YDRFVLYKEWGRD (SEQ ID NO: 82) in LILRA3) and Epitope 2 (sequence SSEWSAPSDPLD (SEQ ID NO: 83) in LILRB1, sequence ECSAPSDPLDI (SEQ ID NO: 84) in LILRB2, and sequence SEWSAPSDPLD (SEQ ID NO: 85) in LILRA3). Binding was also observed to additional putative epitopes: Epitope 3 (sequence LQCVSDVGYD (SEQ ID NO: 86) in LILRB2 and sequence FQCGSDAGYDRF (SEQ ID NO: 87) in LILRA3), and Epitope 4 (sequence FLLTKEGAADDPW (SEQ ID NO: 88) in LILRB1 and sequence AADAPLRLRSIHEY (SEQ ID NO: 89) in LILRB2). Although with a weaker signal, binding to similar peptides was observed for Antibody 1. Antibody 4 was found binding to two putative epitopes; Epitope 5 (sequence RSYGGQYR (SEQ ID NO: 90) in LILRB1 and sequence PVSRSYGGQYRC (SEQ ID NO: 91) in LILRB2). Weak binding to peptide arrays was observed for Antibody 5 suggesting one putative epitope; Epitope 6 sequence LDILIAGQFYD

(SEQ ID NO: 92) in LILRB1, sequence APSDPLDILI (SEQ ID NO: 93) in LILRB2, and sequence PSDPLDILI (SEQ ID NO: 94) in LILRA3). No significant binding to peptide arrays was observed for Antibody 3.

Amino acids may be referred to by their one letter or three letter codes, or by their full name. The one and three letter codes, as well as the full names, of each of the twenty standard amino acids are set out below.

TABLE 5
Amino acids, one and three-letter codes.

| Amino acid | One letter code | Three letter code |
|---|---|---|
| alanine | A | Ala |
| arginine | R | Arg |
| asparagine | N | Asn |
| aspartic acid | D | Asp |
| cysteine | C | Cys |
| glutamic acid | E | Glu |
| glutamine | Q | Gln |
| glycine | G | Gly |
| histidine | H | His |
| isoleucine | I | Ile |
| leucine | L | Leu |
| lysine | K | Lys |
| methionine | M | Met |
| phenylalanine | F | Phe |
| proline | P | Pro |
| serine | S | Ser |
| threonine | T | Thr |
| tryptophan | W | Trp |
| tyrosine | Y | Tyr |
| valine | V | Val |

In preferred embodiments, an antibody or an antigen-binding fragment thereof of the invention may comprise the set of six CDRs of antibody:
(a) Clone 1 (HCDR1 (SEQ ID NO: 1), HCDR2 (SEQ ID NO: 2), HCDR3 (SEQ ID NO: 3), LCDR1 (SEQ ID NO: 4), LCDR2 (SEQ ID NO: 5), and LCDR3 (SEQ ID NO: 6),
(b) Clone 2 (HCDR1 (SEQ ID NO: 9), HCDR2 (SEQ ID NO: 10), HCDR3 (SEQ ID NO: 11), LCDR1 (SEQ ID NO: 12), LCDR2 (SEQ ID NO: 13), and LCDR3 (SEQ ID NO: 14)),
(c) Clone 3 (HCDR1 (SEQ ID NO: 17), HCDR2 (SEQ ID NO: 18), HCDR3 (SEQ ID NO: 19), LCDR1 (SEQ ID NO: 20), LCDR2 (SEQ ID NO: 21), and LCDR3 (SEQ ID NO: 22)),
(d) Clone 4 (HCDR1 (SEQ ID NO: 25), HCDR2 (SEQ ID NO: 26), HCDR3 (SEQ ID NO: 27), LCDR1 (SEQ ID NO: 28), LCDR2 (SEQ ID NO: 29), and LCDR3 (SEQ ID NO: 30)), or,
(e) Clone 5 (HCDR1 (SEQ ID NO: 33), HCDR2 (SEQ ID NO: 34), HCDR3 (SEQ ID NO: 35), LCDR1 (SEQ ID NO: 36), LCDR2 (SEQ ID NO: 37), and LCDR3 (SEQ ID NO: 38)) when defined by Kabat nomenclature).

An antibody or an antigen-binding fragment thereof of the invention may comprise a VH and VL sequence of antibody:
(a) Clone 1 (SEQ ID NO: 7 and SEQ ID NO: 8),
(b) Clone 2 (SEQ ID NO: 15 and SEQ ID NO: 16),
(c) Clone 3 (SEQ ID NO: 23 and SEQ ID NO: 24),
(d) Clone 4 (SEQ ID NO: 31 and SEQ ID NO: 32), or
(e) Clone 5, (SEQ ID NO: 39 and SEQ ID NO: 40).

An antibody or an antigen-binding fragment thereof of the invention may comprise one or more, e.g., 1, 2, 3, 4, 5, 6, 7, 8, 9 or 10 further amino acid modifications in the VH and/or VL sequences, provided that functional properties of the antibody are retained.

A modification may be an amino acid substitution, deletion or insertion. Preferably, the modification is a substitution.

In preferred embodiments in which one or more amino acids are substituted with another amino acid, the substitutions may be conservative substitutions, for example according to the following table. In some embodiments, amino acids in the same category in the middle column are substituted for one another, i.e., a non-polar amino acid is substituted with another non-polar amino acid, for example. In some embodiments, amino acids in the same line in the rightmost column are substituted for one another.

TABLE 6
Amino acids.

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

In some embodiments, substitution(s) may be functionally conservative. That is, in some embodiments the substitution may not affect (or may not substantially affect) one or more functional properties (e.g., binding affinity) of the antibody molecule comprising the substitution as compared to the equivalent unsubstituted antibody molecule.

In a preferred embodiment, an antibody or an antigen-binding fragment thereof of the invention may comprise a VH and/or VL domain sequence with one or more amino acid sequence alterations (addition, deletion, substitution and/or insertion of an amino acid residue), preferably 20 alterations or fewer, 15 alterations or fewer, 10 alterations or fewer, 5 alterations or fewer, 4 alterations or fewer, 3 alterations or fewer, 2 alterations or fewer, or 1 alteration compared with the VH and/or VL sequences of the invention set forth herein.

In a preferred embodiment, an antibody or an antigen-binding fragment thereof of the invention comprises a VH domain amino acid sequence comprising the set of 3 HCDRs of antibody:
(a) Clone 1 HCDR1 (SEQ ID NO: 1), HCDR2 (SEQ ID NO: 2), and HCDR3 (SEQ ID NO: 3); and the VH domain has an amino acid sequence with at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the sequence of Clone 1 set forth in SEQ ID NO: 7;
(b) Clone 2 HCDR1 (SEQ ID NO: 9), HCDR2 (SEQ ID NO: 10), and HCDR3 (SEQ ID NO: 11); and the VH domain has an amino acid sequence with at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the sequence of Clone 2 set forth in SEQ ID NO: 15;
(c) Clone 3 HCDR1 (SEQ ID NO: 17), HCDR2 (SEQ ID NO: 18), and HCDR3 (SEQ ID NO: 19); and the VH domain has an amino acid sequence with at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the sequence of Clone 3 set forth in SEQ ID NO: 23;
(d) Clone 4 HCDR1 (SEQ ID NO: 25), HCDR2 (SEQ ID NO: 26), and HCDR3 (SEQ ID NO: 27); and the VH domain has an amino acid sequence with at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the sequence of Clone 4 set forth in SEQ ID NO: 31; or
(e) Clone 5 HCDR1 (SEQ ID NO: 33), HCDR2 (SEQ ID NO: 34), and HCDR3 (SEQ ID NO: 35) and the VH domain has an amino acid sequence with at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the sequence of Clone 5 set forth in SEQ ID NO: 39;
wherein the sequences are defined by Kabat nomenclature.

In preferred embodiments, an antibody or an antigen-binding fragment thereof of the invention may comprise a VH domain sequence of antibody:
(a) Clone 1 set forth in SEQ ID NO: 7, or a VH domain with an amino acid sequence which has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto;
(b) Clone 2 set forth in SEQ ID NO: 15, or a VH domain with an amino acid sequence which has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto;
(c) Clone 3 set forth in SEQ ID NO: 23, or a VH domain with an amino acid sequence which has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto;
(d) Clone 4 set forth in SEQ ID NO: 31, or a VH domain with an amino acid sequence which has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto; or
(e) Clone 5 set forth in SEQ ID NO: 39, or a VH domain with an amino acid sequence which has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto;
wherein the sequence are defined by Kabat nomenclature.

In a preferred embodiment, an antibody or an antigen-binding fragment thereof of the invention comprises a VL domain amino acid sequence comprising the set of 3 LCDRs of antibody:
(a) Clone 1 LCDR1 (SEQ ID NO: 4), LCDR2 (SEQ ID NO: 5) and LCDR3 (SEQ ID NO: 6); and the VL domain has an amino acid sequence with at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the sequence of Clone 1 set forth in SEQ ID NO: 8;
(b) Clone 2 LCDR1 (SEQ ID NO: 12), LCDR2 (SEQ ID NO: 13) and LCDR3 (SEQ ID NO: 14); and the VL domain has an amino acid sequence with at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the sequence of Clone 2 set forth in SEQ ID NO: 16;
(c) Clone 3 LCDR1 (SEQ ID NO: 20), LCDR2 (SEQ ID NO: 21) and LCDR3 (SEQ ID NO: 22); and the VL domain has an amino acid sequence with at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the sequence of Clone 3 set forth in SEQ ID NO: 24;
(d) Clone 4 LCDR1 (SEQ ID NO: 28), LCDR2 (SEQ ID NO: 29) and LCDR3 (SEQ ID NO: 30); and the VL domain has an amino acid sequence with at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the sequence of Clone 4 set forth in SEQ ID NO: 32; or
(e) Clone 5 LCDR1 (SEQ ID NO: 36), LCDR2 (SEQ ID NO: 37) and LCDR3 (SEQ ID NO: 38); and the VL domain has an amino acid sequence with at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the sequence of Clone 5 set forth in SEQ ID NO: 40;
wherein the sequences are defined by Kabat nomenclature.

In preferred embodiments, an antibody or an antigen-binding fragment thereof of the invention may comprise a VL domain sequence of antibody:
(a) Clone 1 set forth in SEQ ID NO: 8, or a VL domain with an amino acid sequence which has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto;
(b) Clone 2 set forth in SEQ ID NO: 16, or a VL domain with an amino acid sequence which has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto;
(c) Clone 3 set forth in SEQ ID NO: 24, or a VL domain with an amino acid sequence which has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto;
(d) Clone 4 set forth in SEQ ID NO: 32, or a VL domain with an amino acid sequence which has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto; or
(e) Clone 5 set forth in SEQ ID NO: 40, or a VL domain with an amino acid sequence which has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto;
wherein the sequence are defined by Kabat nomenclature.

In a preferred embodiment, an antibody or an antigen-binding fragment thereof of the invention comprises VH and VL domain amino acid sequences comprising the set of 6 HCDRs LCDRs of antibody
(a) Clone 1 HCDR1 (SEQ ID NO: 1), HCDR2 (SEQ ID NO: 2), HCDR3 (SEQ ID NO: 3), LCDR1 (SEQ ID NO: 4), LCDR2 (SEQ ID NO: 5) and LCDR3 (SEQ ID NO: 6);
(b) Clone 2 HCDR1 (SEQ ID NO: 9), HCDR2 (SEQ ID NO: 10), HCDR3 (SEQ ID NO: 11), LCDR1 (SEQ ID NO: 12), LCDR2 (SEQ ID NO: 13) and LCDR3 (SEQ ID NO: 14);
(c) Clone 3 HCDR1 (SEQ ID NO: 17), HCDR2 (SEQ ID NO: 18), HCDR3 (SEQ ID NO: 19), LCDR1 (SEQ ID NO: 20), LCDR2 (SEQ ID NO: 21) and LCDR3 (SEQ ID NO: 22);
(d) Clone 4 HCDR1 (SEQ ID NO: 25), HCDR2 (SEQ ID NO: 26), HCDR3 (SEQ ID NO: 27), LCDR1 (SEQ ID NO: 28), LCDR2 (SEQ ID NO: 29) and LCDR3 (SEQ ID NO: 30); or
(e) Clone 5 HCDR1 (SEQ ID NO: 33), HCDR2 (SEQ ID NO: 34), HCDR3 (SEQ ID NO: 35), LCDR1 (SEQ ID NO: 36), LCDR2 (SEQ ID NO: 37) and LCDR3 (SEQ ID NO: 38);
wherein the sequences are defined by Kabat nomenclature.

In preferred embodiments, an antibody or an antigen-binding fragment thereof of the invention may comprise a VH and VL domain sequence of antibody:
(a) Clone 1 set forth in SEQ ID NO: 7 and 8;
(b) Clone 2 set forth in SEQ ID NO: 15 and 16;
(c) Clone 3 set forth in SEQ ID NO: 23 and 24;
(d) Clone 4 set forth in SEQ ID NO: 31 and 30; or
(e) Clone 5 set forth in SEQ ID NO: 39 and 40;
wherein the sequence are defined by Kabat nomenclature.

The terms “Antibody Clone”, “Clone” and “Antibody”, (e.g. Clone 1, or Antibody 1), are used interchangeably herein to denote Antibodies 1 to 5 of the invention.

Sequence identity is commonly defined with reference to the algorithm GAP (Wisconsin GCG package, Accelerys Inc, San Diego USA). GAP uses the Needleman and Wunsch algorithm to align two complete sequences, maximising the number of matches and minimising the number of gaps. Generally, default parameters are used, with a gap creation penalty equaling 12 and a gap extension penalty equaling 4. Use of GAP may be preferred but other algorithms may be used, e.g. BLAST (which uses the method of Altschul et al. (1990) J. Mol. Biol. 215:405-410), FASTA (which uses the method of Pearson and Lipman (1988) PNAS USA 85:2444-2448), or the Smith-Waterman algorithm (Smith and Waterman (1981) J. Mol Biol. 147:195-197), or the TBLASTN program, of Altschul et al. (1990) supra, generally employing default parameters. In particular, the psi-Blast algorithm may be used (Nucl. Acids Res. (1997) 25 3389-3402). Sequence identity may be defined using the Bioedit, ClustalW algorithm.

The antibody may comprise a CH2 domain. The CH2 domain is preferably located at the N-terminus of the CH3 domain, as in the case in a human IgG molecule. The CH2 domain of the antibody is preferably the CH2 domain of human IgG1, IgG2, IgG3, or IgG4, more preferably the CH2 domain of human IgG1. The sequences of human IgG domains are known in the art.

The antibody may comprise an immunoglobulin hinge region, or part thereof, at the N-terminus of the CH2 domain. The immunoglobulin hinge region allows the two CH2-CH3 domain sequences to associate and form a dimer. Preferably, the hinge region, or part thereof, is a human IgG1, IgG2, IgG3 or IgG4 hinge region, or part thereof. More preferably, the hinge region, or part thereof, is an IgG1 hinge region, or part thereof.

The sequence of the CH3 domain is not particularly limited. Preferably, the CH3 domain is a human immunoglobulin G domain, such as a human IgG1, IgG2, IgG3, or IgG4 CH3 domain, most preferably a human IgG1 CH3 domain.

An antibody of the invention may comprise a human IgG1, IgG2, IgG3, or IgG4 constant region or an engineered version thereof. The sequences of human IgG1, IgG2, IgG3, or IgG4 CH3 domains are known in the art. An antibody of the invention may comprise a human IgG constant region, e.g., a human IgG1 constant region.

An antibody of the invention may comprise a human IgG Fc with effector function. Antibodies of the invention may comprise an Fc with effector function, enhanced effector function, with reduced effector function or with no effector function.

Fc receptors (FcRs) are key immune regulatory receptors connecting the antibody mediated (humoral) immune response to cellular effector functions. Receptors for all classes of immunoglobulins have been identified, including FcγR (IgG), FcεRI (IgE), FcαRI (IgA), FcμR (IgM) and FcδR (IgD). There are three classes of receptors for human IgG found on leukocytes: CD64 (FcγRI), CD32 (FcγRIIa, FcγRIIb and FcγRIIc) and CD16 (FcγRIIIa and FcγRIIIb). FcγRI is classed as a high affinity receptor (nanomolar range KD) while FcγRII and FcγRIII are low to intermediate affinity (micromolar range KD).

In antibody dependent cellular cytotoxicity (ADCC), FcγRs on the surface of effector cells (natural killer cells, macrophages, monocytes and eosinophils) bind to the Fc region of an IgG which itself is bound to a target cell. Upon binding a signalling pathway is triggered which results in the secretion of various substances, such as lytic enzymes, perforin, granzymes and tumour necrosis factor, which mediate in the destruction of the target cell. The level of ADCC effector function various for IgG subtypes. Although this is dependent on the allotype and specific FcγR in simple terms ADCC effector function is high for human IgG1 and IgG3, and low for IgG2 and IgG4. See below for IgG subtype variation in effector functions, ranked in decreasing potency.

TABLE 7
IgG subtype variation in effector functions,
ranked in decreasing potency.

| Effector Function | Species | IgG Subtype Potency |
|---|---|---|
| ADCC | Human | IgG1 ≥ IgG3 >> IgG4 > IgG2 |
| | Mouse | IgG2b > IgG2a > IgG1 >> IgG3 |
| C1q Binding | Human | IgG3 > IgG1 >> IgG2 > IgG4 |
| | Mouse | IgG2a ≥ IgG2b > IgG3 > IgG1 |

FcγRs bind to IgG asymmetrically across the hinge and upper CH2 region. Knowledge of the binding site has resulted in engineering efforts to modulate IgG effector functions

The potency of antibodies can be increased by enhancement of the ability to mediate cellular cytotoxicity functions, such as antibody-dependent cell-mediated cytotoxicity (ADCC) and antibody-dependent cell-mediated phagocytosis (ADCP). A number of mutations within the Fc domain have been identified that either directly or indirectly enhance binding of Fc receptors and significantly enhance cellular cytotoxicity: the mutations S239D/A330L/1332E (“3M”), F243L or G236A. Alternatively enhancement of effector function can be achieved by modifying the glycosylation of the Fc domain, FcγRs interact with the carbohydrates on the CH2 domain and the glycan composition has a substantial effect on effector function activity.

Afucosylated (non-fucosylated) antibodies, exhibit greatly enhanced ADCC activity through increased binding to FcγRIIIa.

Activation of ADCC and CDC may be desirable for some therapeutic antibodies, however, in some embodiments, an antibody that does not activate effector functions is preferred.

Due to their lack of effector functions, IgG4 antibodies are the preferred IgG subclass for receptor blocking without cell depletion. However IgG4 molecules can exchange half-molecules in a dynamic process termed Fab-arm exchange. This phenomenon can occur between therapeutic antibodies and endogenous IgG4. The S228P mutation has been shown to prevent this recombination process allowing the design of IgG4 antibodies with a reduced propensity for Fab-arm exchange.

Fc engineering approaches have been used to determine the key interaction sites for the IgG1 Fc domain with Fcγ receptors and C1q and then mutate these positions to reduce or abolish binding. Through alanine scanning the binding site of C1q to a region covering the hinge and upper CH2 of the Fc domain was identified. The CH2 domain of an antibody or fragment of the invention may comprise one or more mutations to decrease or abrogate binding of the CH2 domain to one or more Fcγ receptors, such as FcγRI, FcγRIIa, FcγRIIb, FcγRIII and/or to complement. CH2 domains of human IgG domains normally bind to Fcγ receptors and complement, decreased binding to Fcγ receptors is expected to decrease antibody-dependent cell-mediated cytotoxicity (ADCC) and decreased binding to complement is expected to decrease the complement-dependent cytotoxicity (CDC) activity of the antibody molecule. Mutations to decrease or abrogate binding of the CH2 domain to one or more Fcγ receptors and/or complement are known in the art. An antibody molecule of the invention may comprise an Fc with modifications K322A/L234A/L235A or L234F/L235E/P331S (“TM”), which almost completely abolish FcγR and C1q binding. An antibody molecule of the invention may comprise a CH2 domain, wherein the CH2 domain comprises alanine residues at EU positions 234 and 235 (positions 1.3 and 1.2 by IMGT numbering) (“LALA mutation”). Furthermore, complement activation and ADCC can be decreased by mutation of Pro329 (position according to EU numbering), e.g., to either P329A or P329G. The antibody molecule of the invention may comprise a CH2 domain, wherein the CH2 domain comprises alanine residues at EU positions 234 and 235 (positions 1.3 and 1.2 by IMGT numbering) and an alanine (LALA-PA) or glycine (LALA-PG) at EU position 329 (position 114 by IMGT numbering). Additionally or alternatively an antibody molecule of the invention may comprise an alanine, glutamine or glycine at EU position 297 (position 84.4 by IMGT numbering).

Modification of glycosylation on asparagine 297 of the Fc domain, which is known to be required for optimal FcR interaction may confer a loss of binding to FcRs; a loss of binding to FcRs has been observed in N297 point mutations. An antibody molecule of the invention may comprise an Fc with an N297A, N297G or N297Q mutation. An antibody molecule of the invention with an aglycosyl Fc domain may be obtained by enzymatic deglycosylation, by recombinant expression in the presence of a glycosylation inhibitor, or following the expression of Fc domains in bacteria.

IgG naturally persists for a prolonged period in the serum due to FcRn-mediated recycling, giving it a typical half-life of approximately 21 days. Half-life can be extended by engineering the pH-dependent interaction of the Fc domain with FcRn to increase affinity at pH 6.0 while retaining minimal binding at pH 7.4. The T250Q/M428L variant, conferred an approximately 2-fold increase in IgG half-life (assessed in rhesus monkeys), while the M252Y/S254T/T256E variant (“YTE”), gave an approximately 4-fold increase in IgG half-life (assessed in cynomolgus monkeys). Extending half-life may allow the possibility of decreasing administration frequency, while maintaining or improving efficacy.

Immunoglobulins are known to have a modular architecture comprising discrete domains, which can be combined in a multitude of different ways to create multispecific, e.g. bispecific, trispecific, or tetraspecific antibody formats. Exemplary multispecific antibody formats are described in Spiess et al. (2015) Mol Immunol 67:95-106 and Kontermann (2012) Mabs 4 (2): 182-97, for example. The antibodies of the invention may be employed in such multispecific formats.

The invention provides an antibody or antigen-binding fragment thereof, such as a human antibody or an antigen-binding fragment thereof, capable of competing with an antibody of the invention described herein (e.g., comprising a set of HCDR and LCDRs of Clone 1, 2, 3, 4, or 5 when defined by Kabat nomenclature and/or the VH and VL amino acid sequences of Clone 1, 2, 3, 4, or 5), for binding to an epitope of human LILRB1, human LILRB2 and/or human LILRA3.

Competition assays include cell-based and cell-free binding assays including an immunoassay such as ELISA, HTRF, flow cytometry, fluorescent microvolume assay technology (FMAT) assay, Mirrorball, high content imaging based fluorescent immunoassays, radioligand binding assays, bio-layer interferometry (BLI), surface plasmon resonance (SPR) and thermal shift assays.

An antibody that binds to the same epitope as, or an epitope overlapping with, a reference antibody refers to an antibody that blocks binding of the reference antibody to its binding partner (e.g., an antigen or “target”) in a competition assay by 50% or more, and/or conversely, the reference antibody blocks binding of the antibody to its binding partner in a competition assay by 50% or more. Such antibodies are said to compete for binding to an epitope of interest.

An antigen-binding protein, such as an antibody or antigen-binding fragment thereof of the invention may be conjugated to a detectable label (for example, a radioisotope); or to a bioactive molecule. In this case, the antigen-binding protein, such as an antibody or antigen-binding fragment thereof may be referred to as a conjugate. Such conjugates may find application in the treatment and/or diagnosis of diseases as described herein.

The antigen-binding proteins of the invention (including conjugates) may be useful in the detection (e.g., in vitro detection) of an epitope bound by an antibody of the invention (an epitope present on human LILRB1, LILRB2, and LILRA3, preferably an epitope present on human LILRB1, LILRB2, LILRB3, LILRA1, LILRA3, LILRA4 and LILRA6). Thus, the present invention relates to the use of an antigen-binding protein of the invention for detecting the presence of epitope bound by an antibody of the invention in a sample. The antigen-binding protein may be conjugated to a detectable label as described elsewhere herein.

In a preferred embodiment, the present invention relates to an in vitro method of detecting an epitope of the invention in a sample, wherein the method comprises incubating an antigen-binding protein of the invention with a sample of interest, and determining binding of the antigen-binding protein to an epitope of the invention present in the sample, wherein binding of the antigen-binding protein indicates the presence of an epitope of the invention in the sample. Methods for detecting binding of an antigen-binding protein to its target antigen are known in the art and include ELISA, ICC, IHC, immunofluorescence, western blot, IP, SPR and flow cytometry.

The sample of interest may be a sample obtained from an individual. The individual may be human. Samples include, but are not limited to, tissue such as tumour tissue, tumour lysates, primary or cultured cells or cell lines, cell supernatants, cell lysates, cerebro-spinal fluid (CSF), platelets, serum, plasma, vitreous fluid, lymph fluid, synovial fluid, follicular fluid, seminal fluid, amniotic fluid, milk, whole blood, plasma, serum, blood-derived cells, urine, saliva, sputum, tears, perspiration, mucus, and tissue culture medium, tissue extracts such as homogenized tissue, tumour tissue, cellular extracts, and combinations thereof.

Following incubation, antigen-binding protein to antigen binding, e.g., antibody to antigen binding, is detected using an appropriate detection system. The method of detection can be direct or indirect, and may generate a fluorescent or chromogenic signal. Direct detection involves the use of primary antibodies that are directly conjugated to a label. Indirect detection methods employ a labelled secondary antibody raised against the primary antigen-binding protein, e.g., antibody, host species. Indirect methods may include amplification steps to increase signal intensity. Commonly used labels for the visualization (i.e., detection) of antigen-binding protein-antigen (e.g., antibody-epitope) interactions include fluorophores and enzymes that convert soluble substrates into insoluble, chromogenic end products.

The term “detecting” is used herein in the broadest sense to include both qualitative and quantitative measurements of a target molecule. Detecting includes identifying the mere presence of the target molecule in a sample as well as determining whether the target molecule is present in the sample at detectable levels. Detecting may be direct or indirect.

Suitable detectable labels which may be conjugated to antigen-binding proteins, such as antibodies, are known in the art and include radioisotopes such as iodine-125, iodine-131, yttrium-90, indium-111 and technetium-99; fluorochromes, such as fluorescein, rhodamine, phycoerythrin, Texas Red and cyanine dye derivatives for example, Cy7, Alexa750 and Alexa Fluor 647; chromogenic dyes, such as diaminobenzidine; latex beads; enzyme labels such as horseradish peroxidase; 38ioinfor or laser dyes with spectrally isolated absorption or emission characteristics; electro-chemiluminescent labels, such as SULFO-TAG which may be detected via stimulation with electricity in an appropriate chemical environment; and chemical moieties, such as biotin, which may be detected via binding to a specific cognate detectable moiety, e.g., labelled avidin or streptavidin.

An antigen-binding protein, such as an antibody or fragment thereof, of the invention may be conjugated to the detectable label by means of any suitable covalent or non-covalent linkage, such as a disulphide or peptide bond. Suitable peptide linkers are known in the art and may be 5 to 25, 5 to 20, 5 to 15, 10 to 25, 10 to 20, or 10 to 15 amino acids in length.

The invention also provides a nucleic acid or set of nucleic acids encoding an antibody or antigen-binding fragment of the invention, as well as a vector comprising such a nucleic acid or set of nucleic acids. Where the nucleic acid encodes the VH and VL domain, or heavy and light chain, of an antibody molecule of the invention, the two domains or chains may be encoded on the same or on separate nucleic acid molecules.

An isolated nucleic acid molecule may be used to express an antibody molecule of the invention. The nucleic acid will generally be provided in the form of a recombinant vector for expression. Another aspect of the invention thus provides a vector comprising a nucleic acid as described above. Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the nucleic acid in a host cell. Vectors may be plasmids, viral e.g. phage, or phagemid, as appropriate.

A nucleic acid molecule or vector as described herein may be introduced into a host cell. Techniques for the introduction of nucleic acid or vectors into host cells are well established in the art and any suitable technique may be employed. A range of host cells suitable for the production of recombinant antibody molecules are known in the art, and include bacterial, yeast, insect or mammalian host cells. A preferred host cell is a mammalian cell, such as a CHO, NS0, or HEK cell, for example a HEK293 cell.

A recombinant host cell comprising a nucleic acid or the vector of the invention is also provided. Such a recombinant host cell may be used to produce an antigen-binding protein (e.g., antibody) of the invention. Thus, also provided is a method of producing an antigen-binding protein, e.g., antibody, of the invention, the method comprising culturing the recombinant host cell under conditions suitable for production of the antigen-binding protein, e.g., antibody. The method may further comprise a step of isolating and/or purifying the antigen-binding protein, e.g., antibody.

Thus the invention provides a method of producing an antigen-binding protein, e.g., antibody, of the invention comprising expressing a nucleic acid encoding the antigen-binding protein, e.g., antibody, in a host cell and optionally isolating and/or purifying the antigen-binding protein, e.g., antibody, thus produced. Methods for culturing host cells are well-known in the art. Techniques for the purification of recombinant antigen-binding proteins, e.g., antibodies, are well-known in the art and include, for example HPLC, FPLC or affinity chromatography, e.g., using Protein A or Protein L. In some embodiments, purification may be performed using an affinity tag on an antigen-binding protein, e.g., antibody. The method may also comprise formulating the antigen-binding protein, e.g., antibody, into a pharmaceutical composition, optionally with a pharmaceutically acceptable excipient or other substance as described below.

Antigen-binding proteins, e.g., antibodies, of the invention are expected to find application in therapeutic applications, in particular therapeutic applications in humans, for example in the treatment of cancer including but not limited to
(i) Acute Myeloid Leukemia (AML), Bladder Urothelial Carcinoma (BLCA), Brain Lower Grade Glioma (LGG), Breast invasive carcinoma (BRCA), Esophageal carcinoma (ESCA), Glioblastoma multiforme (GBM), Head and Neck squamous cell carcinoma (HNSC), Kidney renal clear cell carcinoma (KIRC), Kidney renal papillary cell carcinoma (KIRP), Liver hepatocellular carcinoma (LIHC), Lung adenocarcinoma (LUAD), Lung squamous cell carcinoma (LUSC), Pancreatic adenocarcinoma (PAAD), Sarcoma (SARC), Skin Cutaneous Melanoma (SKCM), Stomach adenocarcinoma (STAD), Testicular Germ Cell Tumours (TGCT), Thymoma (THYM), Thyroid carcinoma (THCA), Uterine Carcinosarcoma (UCS), Uterine Corpus Endometrial Carcinoma (UCEC), Uveal Melanoma (UVM), colorectal cancer, prostate cancer, pediatric cancers, lymphomas and leukemias such as DLBCL, NHL, multiple myeloma, Hodgkins lymphoma;
(ii) cancer positive for LILRB1 or LILRB2 or both LILRB1 and LILRB2;
(iii) cancer positive for immunosuppressive macrophages (as measured by CD163 or CD68 positivity) and/or tumour infiltrating T cells;
(iv) cancer with increased or decreased expression of classical or non-classical MHC Class I
(vi) cancer positive for one or more of LILRB1, LILRB2, LILRB3, LILRA1, LILRA3, LILRA4 and LILRA6.

Also provided is a composition, such as a pharmaceutical composition, comprising an antigen-binding protein, e.g., antibody, according to the invention and an excipient, such as a pharmaceutically acceptable excipient.

The invention further provides an antigen-binding protein, e.g., antibody, of the invention, for use in a method of treatment. Also provided is a method of treating a patient, wherein the method comprises administering to the patient a therapeutically-effective amount of an antigen-binding protein, e.g., antibody, according to the invention. Further provided is the use of an antigen-binding protein, e.g., antibody, according to the invention for use in the manufacture of a medicament. A patient, as referred to herein, is preferably a human patient.

The invention also provides an antigen-binding protein, e.g., antibody, of the invention, for use in a method of treating a cancer in a patient.

Also provided is a method of treating a cancer, such as breast cancer, in a patient, wherein the method comprises administering to the patient a therapeutically-effective amount of an antigen-binding protein, e.g., antibody, according to the invention.

Further provided is the use of an antigen-binding protein, e.g., antibody, according to the invention for use in the manufacture of a medicament for the treatment of a cancer, including but not limited to:
(i) Acute Myeloid Leukemia (LAML), Bladder Urothelial Carcinoma (BLCA), Brain Lower Grade Glioma (LGG), Breast invasive carcinoma (BRCA), Esophageal carcinoma (ESCA), Glioblastoma multiforme (GBM), Head and Neck squamous cell carcinoma (HNSC), Kidney renal clear cell carcinoma (KIRC), Kidney renal papillary cell carcinoma (KIRP), Liver hepatocellular carcinoma (LIHC), Lung adenocarcinoma (LUAD), Lung squamous cell carcinoma (LUSC), Pancreatic adenocarcinoma (PAAD), Sarcoma (SARC), Skin Cutaneous Melanoma (SKCM), Stomach adenocarcinoma (STAD), Testicular Gemm Cell Tumours (TGCT), Thymoma (THYM), Thyroid carcinoma (THCA), Uterine Carcinosarcoma (UCS), Uterine Corpus Endometrial Carcinoma (UCEC), Uveal Melanoma (UVM), colorectal cancer, prostate cancer, pediatric cancers, lymphomas and leukemias such as DLBCL, NHL, multiple myeloma, Hodgkin lymphoma;
(ii) cancer positive for LILRB1 or LILRB2 or both LILRB1 and LILRB2;
(iii) cancer positive for immunosuppressive macrophages (as measured by CD163 or CD68 positivity) and/or tumour infiltrating T cells;
(iv) cancer with increased or decreased expression of classical or non-classical MHC Class I;
(v) cancer positive for one or more of LILRB1, LILRB2, LILRB3, LILRA1, LILRA3, LILRA4 and LILRA6.

The treatment may further comprise administering to the patient a second therapy. The second therapy may be administered to the patient simultaneously, separately, or sequentially to the antigen-binding protein, e.g., antibody, of the invention.

In another aspect, the invention relates to an antigen-binding protein, e.g., antibody, of the invention for use in:
a) treating a cancer,
b) delaying progression of a cancer,
c) prolonging the survival of a patient suffering from a cancer,
d) reducing tumour immune evasion,
e. reducing cancer metastasis,
f) reducing resistance to a second therapy,
g) increasing response rate or overall survival following standard of care therapy,
h) decreasing tumour volume prior to surgical resection, and/or,
i) reducing tumour relapse following surgery or neoadjuvant therapy.

The antigen-binding protein, e.g., antibody, as described herein may thus be for use for therapeutic applications, in particular for the treatment of a cancer, including but not limited to:
(i) Acute Myeloid Leukemia (AML), Bladder Urothelial Carcinoma (BLCA), Brain Lower Grade Glioma (LGG), Breast invasive carcinoma (BRCA), Esophageal carcinoma (ESCA), Glioblastoma multiforme (GBM), Head and Neck squamous cell carcinoma (HNSC), Kidney renal clear cell carcinoma (KIRC), Kidney renal papillary cell carcinoma (KIRP), Liver hepatocellular carcinoma (LIHC), Lung adenocarcinoma (LUAD), Lung squamous cell carcinoma (LUSC), Pancreatic adenocarcinoma (PAAD), Sarcoma (SARC), Skin Cutaneous Melanoma (SKCM), Stomach adenocarcinoma (STAD), Testicular Germ Cell Tumours (TGCT), Thymoma (THYM), Thyroid carcinoma (THCA), Uterine Carcinosarcoma (UCS), Uterine Corpus Endometrial Carcinoma (UCEC), Uveal Melanoma (UVM), colorectal cancer, prostate cancer, pediatric cancers, lymphomas and leukemias such as DLBCL, NHL, multiple myeloma, Hodgkin lymphoma;
(ii) cancer positive for LILRB1 or LILRB2 or both LILRB1 and LILRB2;
(iii) cancer positive for immunosuppressive macrophages (as measured by CD163 or CD68 positivity) and/or tumour infiltrating T cells;
(iv) cancer with increased or decreased expression of classical or non-classical MHC Class I;
(v) cancer positive for one or more of LILRB1, LILRB2, LILRB3, LILRA1, LILRA3, LILRA4 and LILRA6.

An antigen-binding protein, e.g., antibody, as described herein may be used in a method of treatment of the human or animal body.

Related aspects of the invention provide;
(i) an antigen-binding protein, e.g., antibody, described herein for use as a medicament,
(ii) an antigen-binding protein, e.g., antibody, described herein for use in a method of treatment of a disease or disorder,
(iii) the use of an antigen-binding protein, e.g., antibody, described herein in the manufacture of a medicament for use in the treatment of a disease or disorder; and,
(iv) a method of treating a disease or disorder in an individual, wherein the method comprises administering to the individual a therapeutically effective amount of an antigen-binding protein, e.g., antibody, as described herein.

The individual may be a patient, preferably a human patient.

Treatment may be any treatment or therapy in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, ameliorating, delaying, abating or arresting one or more symptoms and/or signs of the condition or prolonging survival of an individual or patient beyond that expected in the absence of treatment.

Treatment as a prophylactic measure (i.e., prophylaxis) is also included. For example, an individual susceptible to or at risk of the occurrence of a cancer, such as breast cancer, may be treated as described herein. Such treatment may prevent or delay the occurrence or recurrence of the disease in the individual. A method of treatment as described may comprise administering at least one further treatment to the individual in addition to the antigen-binding protein, e.g., antibody. The antigen-binding protein, e.g., antibody, described herein may thus be administered to an individual alone or in combination with one or more other treatments. When the antigen-binding protein, e.g., antibody, is administered to the individual in combination with another treatment, the additional treatment may be administered to the individual concurrently with, sequentially to, or separately from the administration of the antigen-binding protein, e.g., antibody. Where the additional treatment is administered concurrently with the antigen-binding protein, e.g., antibody, the antigen-binding protein, e.g., antibody, and additional treatment may be administered to the individual as a combined preparation. For example, the additional therapy may be a known therapy or therapeutic agent for the disease to be treated.

Whilst an antigen-binding protein, e.g., antibody, may be administered alone, antigen-binding proteins, e.g., antibodies, will usually be administered in the form of a pharmaceutical composition, which may comprise at least one component in addition to the antigen-binding protein, e.g., antibody. Another aspect of the invention therefore provides a pharmaceutical composition comprising an antigen-binding protein, e.g., antibody, as described herein. A method comprising formulating an antigen-binding protein, e.g., antibody, into a pharmaceutical composition is also provided.

Pharmaceutical compositions may comprise, in addition to the antigen-binding protein, e.g., antibody, a pharmaceutically acceptable excipient, carrier, buffer, stabilizer or other materials well known to those skilled in the art. The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. The precise nature of the carrier or other material will depend on the route of administration, which may be by infusion, injection or any other suitable route, as discussed below.

For parenteral, for example subcutaneous or intravenous administration, e.g., by injection, the pharmaceutical composition comprising the antigen-binding protein, e.g., antibody, may be in the form of a parenterally acceptable aqueous solution which is pyrogen-free and has suitable pH, isotonicity and stability. Those of relevant skill in the art are able to prepare suitable solutions using, for example, isotonic vehicles, such as Sodium Chloride Injection, Ringer's Injection, or Lactated Ringer's Injection. Preservatives, stabilizers, buffers, antioxidants and/or other additives may be employed as required including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagines, histidine, arginine, or lysine; monosaccharides, disaccharides and other carbohydrates including glucose, mannose or dextrins; chelating agents, such as EDTA; sugars, such as sucrose, mannitol, trehalose or sorbitol; salt-forming counter-ions, such as sodium; metal complexes (e.g., Zn-protein complexes); and/or non-ionic surfactants, such as TWEEN™, PLURONICS™ or polyethylene glycol (PEG).

In some embodiments, antigen-binding proteins, e.g., antibodies may be provided in a lyophilised form for reconstitution prior to administration. For example, lyophilised antigen-binding proteins, e.g., antibodies may be reconstituted in sterile water or saline prior to administration to an individual.

Administration may be in a “therapeutically effective amount”, this being sufficient to show benefit to an individual. The actual amount administered, and rate and time-course of administration, will depend on the nature and severity of what is being treated, the particular individual being treated, the clinical condition of the individual, the cause of the disorder, the site of delivery of the composition, the type of antigen-binding protein, e.g., antibody, the method of administration, the scheduling of administration and other factors known to medical practitioners. Prescription of treatment, e.g., decisions on dosage etc., is within the responsibility of general practitioners and other medical doctors, and may depend on the severity of the symptoms and/or progression of a disease being treated. Appropriate doses of antigen-binding protein, e.g., antibodies, are well known in the art. A therapeutically effective amount or suitable dose of an antigen-binding protein, e.g., antibody, can be determined by comparing in vitro activity and in vivo activity in an animal model. Methods for extrapolation of effective dosages in mice and other test animals to humans are known. The precise dose will depend upon a number of factors, including whether the size and location of the area to be treated, and the precise nature of the antigen-binding protein, e.g., antibody.

A typical antibody dose is in the range 100 μg to 1 g for systemic applications, and 1 μg to 1 mg for topical applications. An initial higher loading dose, followed by one or more lower doses, may be administered. This is a dose for a single treatment of an adult individual, which may be proportionally adjusted for children and infants, and also adjusted for other antibody formats in proportion to molecular weight.

Treatments may be repeated at daily, twice-weekly, weekly or monthly intervals, at the discretion of the physician. The treatment schedule for an individual may be dependent on the pharmacokinetic and bioinformatics properties of the antibody composition, the route of administration and the nature of the condition being treated.

Treatment may be periodic, and the period between administrations may be about two weeks or more, e.g., about three weeks or more, about four weeks or more, about once a month or more, about five weeks or more, or about six weeks or more. For example, treatment may be every two to four weeks or every four to eight weeks. Suitable formulations and routes of administration are described above.

In a preferred embodiment, an antibody as described herein may be for use in a method of treating cancer.

### LIST OF FIGURES

FIG. 1. Increased LILRA3 mRNA expression in cancer tissues compared to normal tissue. Analysis performed using The Cancer Genome Atlas (TCGA). Cancer acronyms as used by TCGA data-base and are publicly available.

FIG. 2. LILRB1 and LILRB2 expression in immune cells across cancer types compared to normal tissue resident macrophages. UMAP analysis of Smart-seq2 datasets from Mulder et al. 2021 https://pubmed.ncbi.nlm.nih.gov/34331874/is shown, with immune cell type classification and LILRB1 and LILRB2 status. Top panel: normal healthy, bottom panel: cancer, A: LILRB1 expression, B: LILRB2 expression, C: LILRB1 and LILRB2 expression.

FIG. 3. Mixed LILRB1 and LILRB2 expression in TAMs from melanoma cancer patients. Analysis of LILRB1 and LILRB2 detection in macrophages in pre- and post-anti-PD-1 and/or anti-CTLA-4 therapy treated melanoma patients (n=33) is shown using Smart-seq2 data from Sade-Feldman et al. https://pubmed.ncbi.nlm.nih.gov/30633907/FIG. 4. Binding to domains of human LILRB1. Plates were coated with recombinant full length LILRB1 ectodomain (SEQ ID NO: 41) or a protein comprising domain 1 and 2 of human LILRB1 protein (SEQ ID NO: 42), and then incubated with 10 nM Clones 1-5, Reference Ab 1, and Isotype control. The bound antibodies were detected using HRP-labeled secondary antibodies and the results were plotted on the graph as absorbance measured at 450 nm.

FIG. 5. Binding to LILRB1 over-expressing cell line. HEK293 cells overexpressing full length human LILRB1 (SEQ ID NO: 45) were incubated with various concentrations of test antibodies (Antibody 1 to 5 and Reference Ab 1). The bound antibodies were detected using PE-labeled secondary antibodies and analyzed by flow cytometry. A) Plot showing mean fluorescence intensity (MFI), B) EC50 values calculated using data from panel A.

FIG. 6. Blocking of HLA-G tetramer binding to LILRB1 over-expressing cells. HEK293 cells overexpressing full length human LILRB1 (SEQ ID NO: 45) were incubated with PE-labeled HLA-G tetramers and 500 nM concentrations of test antibodies (Antibody 1 to 5, Reference Antibody 1, and Isotype control). The bound HLA-G was analyzed by flow cytometry and plotted as the mean fluorescence intensity (MFI).

FIG. 7. Binding to LILRB2 over-expressing cell line. HEK293 cells overexpressing full length human LILRB2 (SEQ ID NO: 46) were incubated with various concentrations of test antibodies (Antibody 1 to 5 and Reference Antibody 1). The bound antibodies were detected using PE-labeled secondary antibodies and analyzed by flow cytometry and the plot shows mean fluorescence intensity (MFI).

FIG. 8. Binding to LILRA1 (SEQ ID NO: 47), LILRA2 (SEQ ID NO: 48), and LILRA3 (SEQ ID NO: 49). Plates were coated with recombinant LILRB1, LILRA1, LILRA2, LILRA3, and the control protein, and then incubated with 10 nM of each of Antibody 1 to 5, Reference Ab 1 and Isotype control. The bound antibodies were detected using HRP-labeled secondary antibodies and plotted as absorbance measured at 450 nm.

FIG. 9. Binding to wild type and allelic variants of LILRB1. Plates were coated with recombinant wt and allelic mutants of LILRB1 (L68P-A93T (SEQ ID NO: 50) and I142T-S155I (SEQ ID NO: 51)), and then incubated with 10 nM of each of Antibody 1 to 5, Reference Antibody 1, and Isotype control. The bound antibodies were detected using HRP-labeled secondary antibodies and plotted as absorbance measured at 450 nm.

FIG. 10. Binding to non-human primate LILRB. Plates were coated with cynomolgus or rhesus LILRB protein and then incubated with 10 nM of each of Antibody 1 to 5, Reference Ab 1, and Isotype control. The bound antibodies were detected using HRP-labeled secondary antibodies and plotted as absorbance measured at 450 nm.

FIG. 11. Cytokine release assay. Human PBMCs were incubated with 20 ug/ml of either of test antibodies (Antibody 1-5, Reference and Isotype), or with 100 ng/ml of LPS, or with 10 ug/ml of PHA, or with 15 ug/ml of OCT3 (anti-CD3 antibody) for 24 h. The cytokines were measured in the media: A) INFg, B) IL-1b, C) IL-2, D) IL-6, E) IL-8, F) IL-10, G) IL-17A, H) MIP-1a, and I) TNFa, using Luminex technology.

FIG. 12. Antibody 1 to 5 binding on iPS derived macrophages. A) Graph shows the percentage of cells which are binding to the target compared to IgG4 isotype control. B) Graph shows the MFI (Mean fluorescence intensity) of the cells with the IgG4 background binding levels subtracted, delta fluorescent intensity (delta FI). Antibodies were tested at 10 ug/ml. “Binding” denotes that in this assay a signal obtained for LILRB1 antibodies was at least more than 2 fold higher than that observed for the IgG4 Isotype control.

FIG. 13. Antibody 1 to 5 binding on NK92 cell line. A) Graph shows the percentage of cells which are binding to the target compared to IgG4 isotype control. B) Graph shows the MFI of the cells with the IgG4 background binding levels subtracted delta fluorescent intensity (delta FI). Antibodies were tested at 10 ug/ml. “Binding” denotes that in this assay a signal obtained for LILRB1 antibodies was at least more than 2 fold higher than that observed for the IgG4 Isotype control.

FIG. 14. Antibodies 1 to 5 enhance cancer induced phagocytosis in iPS-derived macrophages. Induction of macrophage phagocytosis in the presence of A) MHC class I negative cell line DLD1, B) JURKAT HLA-ABC positive, HLA-G negative cell line, C) JURKAT HLA-ABC positive, HLA-G positive cell line. iPSC-DM were incubated in the presence of DLD1 and JURKAT cells. 10 μg/ml of our anti-LILRB1/2 or anti-LILRB1 antibodies (1 to 5), Isotype control (IgG4) or anti-LILRB1 Reference Antibody 1 (‘Reference’) were added to the cell culture medium. Next, phagocytosis was measured for 7 hours after treatment by an Incucyte s3 live-cell analysis system. Graphs shows the 6 hours of analysis for DLD1 and 7 hours of analysis for JURKAT. Total detected phagocytosis and percentage of active macrophage population were calculated as area under the curve over the time course (n=6 for 1, 2, 3, 5, reference and Iso) (n=3/4 for 4, Reference and Iso). Results are presented as mean±SD of the area under the curve. Kruskal-Wallis multiple comparison test was used, comparing the treatments versus IgG4. *p<0.05, **p<0.01, ***p<0.001.

FIG. 15. LILRB1 expression on iPS derived macrophages. LILRB1 expression on iPS derived macrophages determined by flow cytometry (Isotype control and LILRB1 stain). N=3.

FIG. 16. Antibodies 1 to 5 enhance GM-CSF and TNFa production after LPS stimulation in iPS-derived macrophages. iPSC-DM were pre-incubated for 1 hour with 10 μg/ml of our LILRB1 antibodies (1 to 5), Isotype control (IgG4) or the reference anti-LILRB1 antibody (Reference Antibody 1). Next, 25 (A) or 1 ng/ml (B, C) of LPS were directly added to the cells and supernatants were collected after 5 hours. A. Graphs show GM-CSF concentration (pg/ml) detected in the supernatant after each treatment, using 25 ng/ml LPS. B. Graphs show GM-CSF concentration (pg/ml) detected in the supernatant after each treatment, using 1 ng/ml LPS. C. Graphs show TNFα concentration (ug/ml) found in the supernatant after each treatment, using 1 ng/ml LPS. Supernatants were diluted 1:100 into the corresponding ELISA diluent for TNFα measurement. Results are presented as mean±SD. A two-way ANOVA with Kruskal-Wallis multiple comparison test comparing the treatments versus IgG4 was performed. *p<0.05, **p<0.01, ***p<0.001.

FIG. 17. Antibodies 1 to 5 do not enhance GM-CSF and TNFα production after LPS stimulation in iPS derived macrophages in the absence of co-stimulation. iPSC-DM were incubated for 6 hours with 10 μg/ml of anti-LILRB1 antibodies 1 to 5, Isotype control (IgG4) or anti-LILRB1 Reference Antibody 1. A. GM-CSF ELISA was performed on undiluted supernatants. Graphs show GM-CSF concentration (pg/ml) detected in the supernatant after each treatment. B. Graphs show TNFα concentration (pg/ml) found in the supernatant after each treatment. Supernatants were diluted 1:50 into the corresponding ELISA diluent for TNFα measurement. Concentrations interpolated from standard curve were multiplied by the dilution factor before plotting. Results are presented as mean±SD. A two-way ANOVA with Kruskal-Wallis multiple comparison test comparing the treatments versus IgG4 was performed. No statistically significant differences were found.

FIG. 18. Antibodies 1 to 5 enhance GM-CSF and TNFa production after LPS stimulation in primary monocyte-derived macrophages. Donor-derived (n=5) monocytes were differentiated into macrophages in vitro exposed to M-CSF. At the end of the culture period (day 8), cells were replated into 96-well plates and left overnight. The next day, the macrophages were exposed to anti-LILRB1 antibodies (1 to 5), isotype control (IgG4) and anti-LILRB1 reference antibody (Reference Antibody 1) for 1 hour and then exposed to LPS at 1 ng/ml (5 h). A. GM-CSF ELISA was performed on undiluted supernatants. Graphs show GM-CSF concentration (pg/ml) detected in the supernatant after each treatment. B. Graphs show TNFα concentration (pg/ml) found in the supernatant after each treatment. Supernatants were diluted 1:10 into the corresponding ELISA diluent for TNFα measurement. Concentrations interpolated from standard curve were multiplied by the dilution factor before plotting. Graph shows pg/mL or ug/mL values for each group. Data are presented as the mean±SD. A two-way ANOVA with Dunn's multiple comparison test comparing the treatments versus IgG4 was performed. *p<0.05.

FIG. 19. Sequence alignment of human LILRB1 (SEQ ID NO: 45), human LILRB2 (SEQ ID NO: 46) and human LILRA3 (SEQ ID NO: 49)

FIG. 20. VH, VL and CDR sequences (Kabat) of Antibody 1 to 5.

FIG. 21. Neutralization of HLA-A, HLA-E, and HLA-G binding to LILRB1 and LILRB2 receptors. Neutralising reference antibodies 1 to 3 (LILRB1 specific, LILRB2 specific, and LILRB1/2 specific, respectively) were used as positive controls.

FIG. 22. Secretion of TNFα by monocyte-derived macrophages under basal (CSF-1 alone) (A) and immune-suppressed, with CSF-1, IL-10 and TGFβ (B), conditions. Graph represents normalized to isotype control results from three independent experiments using MDMs derived from three different donors. Reference 1 is a LILRB1-specific antibody, reference 2 is a LILRB2-specific antibody, and reference 3 antibody has dual LILRB1/LILRB2 specificity.

FIG. 23. (A) Immunogenicity scores for antibody clones 1-5 obtained using Abzena's iTope-AI and TCED™ in silico methodology. (B) Comparison of the scores for Antibodies 1 to 5 (*) with those for other fully human therapeutic antibodies, humanized, chimeric, and murine Abs (●); and for non-human proteins (p).

FIG. 24. (A) Fluorescence intensities for binding to linear peptide microarrays spanning D3-D4 regions of human LILRB1, LILRB2, and LILRA3. (B) Fluorescence intensity for binding to “conformational” peptide microarrays spanning D3-D4 regions of human LILRB1, LILRB2, and LILRA3.

FIG. 25. MDM reprogramming F(ab)′2.

FIG. 26. MDM reprogramming Fab monomers.

FIG. 27. IFN gamma production. The y-axis shows fold change versus CTR. 1. IgG4 isotype, 2. OPDIVO, 3. Rat IgG2a, 4. Reference Antibody 1, 5. Reference Antibody 2; 6 Antibody 2 IgG4, 7, Isotype IgG4, 8. Antibody 1 IgG4, 9. Reference Antibody 3, 10. Antibody 3 IgG4, 11. Antibody 2 IgG1 LALA, 12. Antibody 5 IgG4, 13. Isotype IgG1, 14. Reference Antibody 4, 15. Antibody 2 IgG1, 16. Isotype IgG1 LALA, 17. Antibody 4 IgG4.

FIG. 28. CD4+ T cell proliferation (▴) and expansion (●). The y-axis shows fold change versus CTR. 1. IgG4 isotype, 2. OPDIVO, 3. Rat IgG2a, 4. Reference Antibody 1, 5. Reference Antibody 2; 6 Antibody 2 IgG4, 7, Isotype IgG4, 8. Antibody 1 IgG4, 9. Reference Antibody 3, 10. Antibody 3 IgG4, 11. Antibody 2 IgG1 LALA, 12. Antibody 5 IgG4, 13. Isotype IgG1, 14. Reference Antibody 4, 15. Antibody 2 IgG1, 16. Isotype IgG1 LALA, 17. Antibody 4 IgG4.

FIG. 29. A. Wild type Jurkat cells, anti-LILRB1/2 antibodies enhanced NKL cytolytic activity compared to the isotype control on wild type Jurkat cells. B. Jurkat HLA-G cells. The increase in cytolytic capacity was higher in the HLA-G over-expressing Jurkat co-cultures. Fold change is shown on the y-axis. 1. Untreated. 2. IgG4 isotype. 3. Antibody 1. 4. Antibody 2. 5. Antibody 3. 6. Antibody 4. 7. Antibody 5.

FIG. 30. Anti-LILRB1/2 and reference antibodies enhanced NKL cytolytic activity compared to the isotype control on wild type Jurkat cells. Fold change is shown on the y-axis. 1. Untreated. 2. IgG4 isotype. 3. Reference Antibody 3. 4. Reference Antibody 4. 5. Reference Antibody 5. 6. Reference Antibody 6. 7. Reference Antibody 1. 8. Reference Antibody 2. 9. Antibody 1. 10. Antibody 2. 11. Antibody 3. 12. Antibody 4. 13. Antibody 5.

FIG. 31. Binding of Antibodies 1 to 5 to all LILR family members tested by single point ELISA. Recombinant LILRB1-5 and LILRA1 (A), or LILRB1 and LILRA2-6 (B) were coated on the ELISA plates. The plates were then blocked with milk in PBST. Next, the plates were incubated with 1 nM of Clone 1-5, Reference 1-3, or IgG4 isotype control. Bound antibodies were detected using HRP-conjugated anti-human Fc antibodies and the chromogenic substrate. Antibodies 1 to 5 bound to LILRB1, LILRB2, LILRB3, LILRA1, LILRA3, LILRA4, and LILRA6.

FIG. 32. Binding of Clones 1-5 to selected LILR family members tested by multiple point ELISA. Recombinant LILRB1, LILRB2, LILRB3, LILRA1, LILRA3, LILRA4, and LILRA6, or irrelevant control protein were coated on the ELISA plates. The plates were then blocked with milk in PBST. Next, the plates were incubated with serial dilutions (12.5 nM to 0.02 nM) of Clones 1-5 (A-E). Bound antibodies were detected using HRP-conjugated anti-human Fc antibodies and the chromogenic substrate.

FIG. 33. Cross-competition between Antibody (Clone) 2 and other Abs in ELISA. Plates were coated with human LILRB1 and, after blocking, incubated with 5 nM of biotinylated Clone 2 and different concentrations of unlabelled Clones 1-5 and the Reference 1-3 Abs. Bound biotinylated Clone 2 was detected using HRP-conjugated streptavidin and a chromogenic substrate. Unlabelled Clone 2 competed its biotinylated counterpart with low nM IC₅₀, while Clones 1, 3, 4 and 5 each yielded IC₅₀ of about 10 nM. None of the Reference Abs, nor the isotype control, could compete with Antibody 2 up to 250 nM concentrations. These data indicate that Clone 2 binds in a unique epitope on LILRB1 that is distal from the epitopes of all the Reference Abs, but proximal to the epitopes of Antibodies (Clones) 1, 3, 4, and 5.

FIG. 34. Epitope mapping of Antibody (Clone) 2 on human LILRB1. The antibody was incubated with the recombinant human LILRB1-avi-his in D₂O 20 mM Na Phosphate, 150 mM NaCl, pH 7.4 for different periods of time. The protein mixture was then digested with proteases and analysed on the Leap HDX auto sampler and Waters Cyclic IMS MS machine. (A) The relative changes (lighter less, darker more) in deuterium uptake after 2, 10, and 60 mins (top to bottom bars under the sequence) in proteolytic peptides is indicated by the heatmap below the sequence of the LILRB1. The putative epitope for Clone 2 (sequence AEFPMGPVTSAHAGT (SEQ ID NO: 78)) is underlined with a solid line and the other region possibly involved in the Antibody (Clone) 2 binding (sequence LTHPSDPLEL (SEQ ID NO: 79)) is underlined with a dashed line. (B) Alignment of all 11 human LILR family members with the putative epitope identified by HDX outlined in solid line and the other region possibly involved in the Clone 2 binding is outlined with a dotted line. The LILR family members bound by Clones 1-5 are in dashed line box. (C) Aligned 3D structures (surface view) of human LILRB1 (light grey) and LILRB2 (dark grey) with the putative epitope in domain 4 highlighted black. (D) A ribbon view of LILRB1 (left) and LILRB2 (right) 3D structures with the putative Clone 2 epitope in domain 4 highlighted black

FIG. 35. Antibody (Clone) 2 enhances secretion of TNFα by human blood monocyte derived macrophages (MDMs) upon induction with various stimuli. M0 polarized MDMs were incubated for 1 h with 10 μg/ml of Clone 2 and then stimulated with various compounds: (A) IL-1β at 10 ng/ml, (B) TLR7/TLR8 agonist R848 at 0.5 μg/ml, (C) TLR3 ligand LMW Poly(I:C) at 10 μg/ml, (D) TLR3 ligand HMW Poly(I:C) at 10 μg/ml, (E) STING agonist c-di-AMP at 10 μg/ml, and (F) TRL4 agonistic HMGB1-derived peptide at 30 μg/ml. Data presented are background normalized.

FIG. 36. Antibody (Clone) 2 enhances secretion of TNFα by MDMs polarized to either M0 or M2 phenotype (with IL-10 and TGFβ) co-cultured with A375 melanoma cells upon induction with LPS. MDMs were co-cultured with A375 cells for 4.5h were incubated with 10 μg/ml of Clone 2 for 1 h and then stimulated with LPS (1 ng/ml and 10 ng/ml, for M0 and M2 MDMs, respectively) for 3.5h. Data presented are background normalized.

### EXAMPLES

### Generation of Anti-LILRB1 Antibodies

Immunizations. Fully human antibodies were generated by immunizing ATX transgenic mice Alloy Therapeutics) with human LILRB1 ectodomain (aa 24-459) fused to mouse IgG2a Fc (SEQ ID NO: 41). Animals with the highest antigen-specific serum native titres directed against human LILRB1 and rhesus LILRB proteins were used for hybridoma generation, immune single-chain Fab (scFab) phage library creation, and B-cell sorting. Lymphocytes were obtained from spleen and/or draining lymph nodes. Pooled lymphocytes (from each harvest) were dissociated from lymphoid tissue by grinding in a suitable medium (e.g., Dulbecco's Modified Eagle Medium (DMEM).

Hybridoma Generation and screening. B cells were selected and/or expanded using standard methods, and fused with a suitable fusion partner using techniques that were known in the art. Hybridoma supernatants with binding to human LILRB1 were then selected for further characterization.

Immune scFAb phage library generation and screening. B cells were selected, and variable regions of heavy and light Ab chains were used to construct single chain Fab libraries cloned into a phagemid vector using techniques that were known in the art. The libraries were then panned against human LILRB1-mIgG2a (SEQ ID NO: 41) and/or rhesus LILRB-mIgG2a (SEQ ID NO: 43) proteins. A periplasmic extract of phage clones producing scFab with binding to human LILRB1 were then selected for further characterization.

Antigen-specific B-cell sorting. B cells were selected and stained with human LILRB1-mIgG2a protein (SEQ ID NO: 41) followed by fluorescently-labelled anti-mouse IgG secondary antibodies. B-cells expressing antibodies binding to human LILRB1 were then individually sorted using techniques that were known in the art.

Recovery of anti-LILRB1 antibodies' sequences. Hybridoma and B-cells cell lysates were used to amplify the antibody heavy and light chain variable region (V) genes using cDNA synthesis via reverse transcription, followed by a polymerase chain reaction (RT-PCR). Phagemid DNA preps were used to amplify the antibody heavy and light chain variable region (V) genes using DNA synthesis via a polymerase chain reaction (PCR). Amino acid sequences were deduced from the corresponding nucleic acid sequences bioinformatically. The derived amino acid sequences were then analysed to determine the germline sequence origin of the antibodies and to identify deviations from the germline sequence. The amino acid sequences corresponding to complementary determining regions (CDRs) of the sequenced antibodies were aligned and these alignments were used to group the clones by similarity.

### Biochemical Characterization of Anti-LILRB1 Antibodies

Antibody expression. Antibodies were expressed in CHO suspension cells and purified using Protein-A affinity chromatography, followed by buffer exchange to a phosphate buffer pH7.4.

Binding to human LILRB1 and LILRB2. HEK293 cells stably expressing full length human LILRB1 (SEQ ID NO: 45), or LILRB2 (SEQ ID NO: 46), or wild-type cells were detached from the culture plates using methods known in art. The cells were then incubated with anti-LILRB1 Ab clones at different concentrations followed by incubation with fluorescently labelled secondary antibodies. The extent of antibody binding to cells stably expressing full-length human LILRB1, or full-length human LILRB2, was determined by flow cytometry and quantified using geometric mean of the fluorescent signal (GEOM).

Binding to allelic forms of LILRB1. There are 4 major allelic variants of human LILRB1 protein ectodomain. The variants of LILRB1 were expressed as recombinant proteins comprising a modified LILRB1 ectodomain and mouse IgG2 Fc region (SEQ ID NO: 50 and 51) in suspension CHO cells, and purified by using techniques that were known in the art. These recombinant proteins were then used in the direct enzyme-linked immunosorbent assay (ELISA) to determine the antibodies potential to bind different allelic forms of LILRB1. In short, Maxisorp (Nunc) or similar immune-assay plates were coated with solutions of target proteins in PBS, at 4° C., overnight. Plates were then washed with PBS, blocked with 5% milk in PBS, and washed again. Test antibodies diluted in PBS were then added to the plates and incubated at room temperature for at least 1h. Plates were subsequently washed with PBS/0.05% Tween-20 (PBST) and incubated with a solution of horse radish peroxidase (HRP) labelled secondary antibodies at room temperature for at least 1 h. After washing with PBST and PBS, bound antibodies were detected using the 3,3′,5,5;-tetramethylbenzidine (TMB) chromogenic solution. After 5 to 10 minutes, the reaction was stopped with 1% sulphuric acid and absorbance read at 450 nm.

Binding to non-human primate LILRB protein. Ectodomains of rhesus monkey and of cynomolgus monkey homologs of human LILRB1 and/or LILRB2 proteins were expressed as fusion to mouse IgG2a Fc (SEQ ID NO: 43 and SEQ ID NO: 44) and used to test cross-species reactivity of anti-LILRB1 antibodies in the direct ELISA, as described above.

Binding to LILRA and LILRB family members. To determine the antibodies potential to bind LILRA receptors, recombinant LILRA1 (SEQ ID NO: 47), LILRA2 (SEQ ID NO: 48), and LILRA3 (SEQ ID NO: 49) proteins from commercial sources were used in the direct ELISA method as described above.

Blocking of HLA binding to LILRB1 and to LILRB2. HEK293 cells expressing human LILRB1 receptor or LILRB2 receptor were incubated with fluorescently labelled HLA-A, or HLA-E, or HLA-G oligomers, in the presence or absence of the antibodies, before being analysed by flow cytometry, as described above.

Epitope binning. Cross-neutralization of binding of individual anti-LILRB1 antibodies to LILRB1 fused to mouse IgG2a Fc was investigated using techniques that were known in the art e.g. Surface Plasmon Resonance (SPR). Recombinant human LILRB1 ectodomain fused to mouse IgG2a Fc (SEQ ID NO: 41) was first immobilized on the SPR chip and then two different anti-LILRB1 antibodies were consecutively injected onto the chip. If injection of the second antibody did not lead to the increase of the SPR signal it was concluded that the two antibodies bind to the similar region on LILRB1 molecule.

Antibody Binding to iPSC-Derived Macrophages

iPSC-derived macrophages were detached using enzyme-free, PBS Cell Dissociation buffer (Life Technologies Cat #13151014) according to manufacturer's instructions. Cells were then blocked with MACS FcR Blocking Reagent (Cat #130-059-901) for 20 min on ice according to manufacturer instructions. Cells were washed with PBS 2% BSA, 5 mM EDTA DPBS (FACS Buffer). For each antibody test, 2×10⁵ cells were incubated for 30 min on ice with appropriate test antibodies or IgG4 Isotype control at a concentration of 10 ug/ml. Macrophages were then washed with FACS Buffer once and stained on ice for 30 min with secondary antibody (PE mouse Anti-Human IgG4-pFC′ Southern Biotech Cat #9190-09) at a concentration of 1 ug/ml. Macrophages were then washed once with FACS Buffer and re-suspended in 200 ul of FACS Buffer and DAPI (0.1 ug/ml). Cells were kept on ice prior to data acquisition using LSR Fortessa Analyser (BD). FCS Express was used as analysis software.

### NK92 Binding Assay.

### Procedure:

1. Prepare FACS buffer: 2% BSA, 5 mM EDTA-DPBS
2. Collect cells. If suspension cells are used, collect and re-suspend in FACs buffer for counting, and proceed to step 5.
3. Pipette up and down to detach the cells.
4. Count cells. 2×10⁵ per specimen are required.
5. Spin down cells at 200G for 3 min. If blocking is proceed to step 6, otherwise proceed to step 10.
6. Re-suspend each sample (up to 10⁷ cells in 80 μl of 2% BSA-PBS-EDTA) (Use 1.5 ml Tube).
7. Add 20 μL of FcR Blocking Reagent for <10⁷ total cells
8. Incubate 20 min on ice.
9. Add 1 ml of FACS buffer and spin down at 200G for 3 min.
10. Aspirate supernatant and add FACS buffer volume, so cells end up being 200,000 in 100 μl (2×10⁶ cells/ml).
11. Take a 96 U-Bottom well plate and label: a) Unstained b) Isotype Control c) Test Antibody.
12. Place 200,000 cells per well (100 μl).
13. Add corresponding primary antibody (fill in table below), and mix.
14. Incubate 30 min at 4° C. in the dark.
15. Add 100 μl of FACS buffer to each well.
16. Spin down cells 200G for 3 min and remove supernatant. If secondary antibody is used proceed to step
17, otherwise proceed to step 23.
17. Re-suspend cells in 100 μl of FACS Buffer.
18. Add corresponding secondary antibody (fill in table below), and mix. * Note, some secondary antibodies need to be mixed beforehand.
19. Incubate 30 min at 4° C. in the dark.
20. Add 100 μl of FACS buffer to each well.
21. Spin down cells 200 g for 3 min.
22. Remove supernatant.
23. Re-suspend in 200 μL of FACS Buffer.
24. Add DAPI (1:1000, 0.2 μl) to each well.
25. Move each sample to FACS Tube.
26. Bring to Flow-Cytometer.

TABLE 8
List of antibody concentrations for NK92 binding assay.

| | | Final | | ul to add | | | | |
|---|---|---|---|---|---|---|---|---|
| Antibody | Fluorochrome | concentration | | in 100 ul | Concentration | | Isotype | Notes |
| Secondary (Anti- | PE | 1 | ug/ml | 1 | 100 | ug/ml | N/A | mixed before |
| IgG4-FC) | | | | | | | | addition to cells |
| IgG4 Isotype | N/A | 10 | ug/ml | 1 | 1000 | ug/ml | N/A | |
| TEST antibody | N/A | 10 | ug/ml | 4 | 250 | ug/ml | IgG4 | |

iPSC Methodology for Production of Genetically Engineered Human iPSC-Derived Macrophages.

WT iPSC 55 line published protocol: https://www.jove.com/t/61038/production-characterization-human-macrophages-from-pluripotent-stern

Production and Characterization of Human Macrophages from Pluripotent Stem Cells

Lopez-Yrigoyen et al. (2020), M., May, A., Ventura, T., Taylor, H., Fidanza, A., Cassetta, L., Pollard, J. W., Forrester, L. M. Production and Characterization of Human Macrophages from Pluripotent Stem Cells. J. Vis. Exp. (158), e61038, doi: 10.3791/61038 (2020).

Generation of macrophages from human induced pluripotent stem cells, and methods for their subsequent characterization were performed as described in Lopez-Yrigoyen et al., (2020). Cell surface marker expression, gene expression, and functional assays were used to assess the phenotype and function of these iPSC-derived macrophages. The optimized protocol described in Lopez-Yrigoyen et al., (2020). allowed for the generation of macrophages from human induced pluripotent stem cells (iPSCs) in vitro. These iPSC-derived macrophages (iPSC-DMs) expressed human macrophage cell surface markers, including CD45, 25F9, CD163, and CD169, and a live-cell imaging functional assay demonstrated that they are capable of exhibiting robust phagocytic activity. Cultured iPSC-DMs can be activated to different macrophage states that display altered gene expression and phagocytic activity by the addition of LPS and IFNg, IL4, or IL10, providing a platform to generate human macrophages carrying genetic alterations that model specific human disease and a source of cells for drug screening or cell therapy to treat these diseases.

Serum- and feeder-free protocols for the maintenance, freezing, and thawing of human iPSCs, and for the differentiation of these iPSCs into functional macrophages are described below. The protocol is very similar to that described by Van Wilgenburg et al. ( ), with minor alterations including: 1) iPSC-maintenance media; 2) ROCK inhibitor was not used in the EB formation stage; 3) a mechanical approach rather than an enzymatic approach is used to generate uniform Ebs from iPSC colonies; 4) the method for EB harvest and plating down was different; 6) suspension cells were harvested 2× a week, rather than weekly; and 6) harvested suspension cells were cultured under CSF1 for macrophage maturation for 9 days rather than 7 days. The protocols used to characterize iPSC-derived macrophage phenotype and function included analyses for gene expression (qRT-PCR), cell surface marker expression (flow cytometry), and functional assays to assess phagocytosis and polarization.

### Protocol

NOTE: All reagents and equipment used in this protocol are listed in Table of Materials. Media should be at 37° C. for cell culture. Media and reagents used in the differentiation protocol must be sterile.

TABLE 9
Table of Materials

| | | Catalog | |
|---|---|---|---|
| Name | Company | Number | Comments |
| 2-Mercaptoethanol (50 mM) | Invitrogen | 31350010 | |
| Abtibody CD64-APC-CY7 | Biolegend | 305026 | Dilution factor: 1:100 |
| Antibody 25F9-EFLUOR 660 | Ebioscience | 15599866 | Dilution factor: 1:20 |
| Antibody CCR2-PE-Cy7 | Biolegend | 357212 | Dilution factor: 1:100 |
| Antibody CCR5 PE | Biolegend | 313707 | Dilution factor: 1:100 |
| Antibody CCR8 PE | Biolegend | 360603 | Dilution factor: 1:100 |
| Antibody CD11b-PE | Biolegend | 301305 | Dilution factor: 1:50 |
| Antibody CD14-APC | Ebioscience | 10669167 | Dilution factor: 1:20 |
| Antibody CD163-PE-CY7 | Biolegend | 333614 | Dilution factor: 1:25 |
| Antibody CD169-APC | Biolegend | 346007 | Dilution factor: 1:25 |
| Antibody CD206-PE | Biolegend | 321106 | Dilution factor: 1:100 |
| Antibody CD209-PE-CY7 | Biolegend | 3310114 | Dilution factor: 1:100 |
| Antibody CD274-PE-CY7 | Biolegend | 329718 | Dilution factor: 1:100 |
| Antibody CD43-PE | Ebioscience | 10854419 | Dilution factor: 1:100 |
| Antibody CD45-APC | Ebioscience | 15577936 | Dilution factor: 1:20 |
| Antibody CD86-APC | Biolegend | 305412 | Dilution factor: 1:100 |
| Antibody CD93-PE | Ebioscience | 10804637 | Dilution factor: 1:100 |
| Antibody CX3CR1-PE | Biolegend | 307650 | Dilution factor: 1:100 |
| Antibody HLA-DR-BV650 | Biolegend | 307650 | Dilution factor: 1:100 |
| Antiboy CD115-PE | Biolegend | 347308 | Dilution factor: 1:40 |
| Cell Dissociation Buffer, enzyme free | Thermofisher | 13151014 | |
| Cell Dissociation Buffer, enzyme-free, PBS | Gibco | 13151014 | |
| CellCarrier-96 Ultra Microplates, tissue culture | PerkinElmer | 6055302 | |
| treated, black, 96-well | | | |
| CellMask Deep Red Plasma Membrane Stain | Thermofisher | C10046 | Cryopreservation |
| | | | media |
| Cryostor CS10 | Sigma | C2874 | |
| CTS CELLstart Substrate | Invitrogen | A1014201 | Stem cell substrate |
| DAPI | Merck | D9542- | Final concentration 1 |
| | | 1MG | μg/mL |
| DPBS, calcium, magnesium (500 mL) | Thermofisher | 14040091 | |
| FcR Blocking Reagent, human | MACS Miltenyi | 130-059-901 | |
| | Biotec | | |
| FGF-Basic (AA 10-155) Recombinant Human Protein | Thermofisher | PHG0021 | |
| GlutaMAX Supplement | Thermofisher | 35050061 | |
| Human Recombinant IFNY | Thermofisher | 14-8319-80 | |
| Human Serum Albuminum | Irvine | 9988 | |
| | Scientific | | |
| Lipopolysaccharide (LPS) from E. Coli | Sigma | L2630 | |
| NucBlue (Hoechst33342) | Thermofisher | R37605 | |
| pHrodo Green Zymosan Bioparticles Conjugate for | Thermofisher | P35365 | |
| Phagocytosis | | | |
| Porcine Skin Gelatin | Sigma | G9136 | |
| Recombinant Human BMP4 Protein | R&D | 314-BP-010 | |
| Recombinant Human IL10 | Preprotech | 200-10 | |
| Recombinant Human IL3 | Preprotech | 200-03-10 | |
| Recombinant Human IL4 | Preprotech | 200-04 | |
| Recombinant Human MCSF (carrier-free) 100 μg | Biolegend | 574806 | |
| Recombinant Human VEGF Protein | R&D | 293-VE-010 | |
| Rock Inhibitor Y-27632 | Merck | SCM075 | |
| SCF (C-Kit Ligand) Recombinant Human Protein | Thermofisher | PHC2111 | |
| StemPro hESC SFM | Thermofisher | A1000701 | |
| StemPro EZPassage Disposable Stem Cell | Thermofisher | 23181010 | |
| Passaging Tool | | | |
| Ultralow attachment plates: Cell culture multi-well | Greiner | 657970 | |
| plate, 6 well, cell star cell repellent surface | | | |
| UltraPure 0.5M EDTA, pH 8.0 | Invitrogen | 15575020 | |
| X-Vivo 15 500 mL bottle | Lonza | BE02-060F | |

1. Human iPSC Line Thawing and Maintenance
1. Cell Maintenance Medium, Growth Factors, and Other Reagents were Prepared.

1. hESC-serum free media (hESC-SFM; see Table of Materials) was prepared by supplementing Dulbecco's modified Eagle medium-F12 (DMEM/F12) with hESC supplement, 1.8% w/v bovine serum albumin (BSA), and 0.1 mM 2-mercaptoethanol.

2. Human basic fibroblast growth factor (bFGF) stock solution (10 μg/mL) was prepared by dissolving bFGF in a sterile 0.1% human serum albumin (I)-phosphate buffered saline (PBS) solution. The stock solution was distributed as 200 μL aliquots in cryotubes. Stock solutions were stored at −20° C. up to 1 year. Once thawed, stock bFGF was stored at 4° C. for up to 7 days.

3. Rho kinase inhibitor (ROCK Inhibitor)-Y27632 stock solution (1 mg/mL) was prepared by dissolving it in sterile water. The stock solution was distributed as 50 μL aliquots in cryotubes. Stock solutions were stored at −20° C. up to 1 year. Once thawed, stock ROCK Inhibitor was stored at 4° C. for up to 7 days.

2. Stem cell substrate (see Table of Materials) was diluted 1:50 in Dulbecco's Phosphate Buffered Saline with calcium and magnesium.

3. The diluted stem cell substrate solution was placed on culture plates so the final volume per surface area was 78 μL/cm². To coat the well of a 6 well plate, 750 μL of solution was added.

4. The coated plate was incubated for 1 h in a humified atmosphere at 37° C. and 5% CO₂.

5. The stem cell substrate coating was aspirated and 1 mL of hESC supplemented with 20 ng/ml bFGF and 10 UM ROCK Inhibitor was added.

6. A vial of frozen human iPSC cells was thawed by incubating the vial at 37° C. until thawed and the cells were transfer into 5 mL hESC-SFM media.

7. Cells were centrifuged at 100×g for 3 min.

8. Cell pellets were resuspended in 0.5 mL hESC-SFM supplemented with 20 ng/ml bFGF and 10 μM ROCK Inhibitor. Cells were transferred to the coated well.

9. Cells were cultured for 24 h.

10. Media was changed to hESC-SFM supplemented with 20 ng/ml bFGF, but without ROCK inhibitor.

11. To maintain the cells, the medium was changed every day until the cells reached 80% confluency. Undifferentiated iPSCs usually took 3 to 4 days to reach 80% confluency.

12. Once the cells reached 80% confluency, the cells were passaged.

1. Spent culture medium was replaced with 1.5 mL of fresh hESC-SFM supplemented with 20 ng/ml bFGF (without ROCK inhibitor).

2. The culture vessel was held in one hand and a disposable cell passaging tool (see Table of Materials) was rolled across the plate in one direction (i.e., left to right). All blades in the roller were required to be touching the plate. Uniform pressure was maintained while rolling.

3. Rolling in the same direction was repeated until the whole well had been covered.

4. The culture vessel was rotated 90° and rolling repeated as described in steps 1.12.2 and 1.12.3.

5. The passaging tool was discarded after use.

6. With a sterile pipette, media in the well was used to dislodge cut colonies.

7. Cells were transferred at a 1:4 ratio onto pre-coated stem cell substrate wells (steps 1.2-1.5) to a final media volume (hE-C-SFM supplemented with 20 ng/ml bFGF) of 1.5 mL per well.

2. Human iPSC Line Freezing

1. To freeze iPSC cells, media of a 70%-80% confluent well of a 6 well plate was replaced with hESC-SFM supplemented with 20 ng/mL bFGF and 10 μM ROCK Inhibitor.

2. The well was incubated at 37° C. and 5% CO₂ for 1 h.

3. Colonies were cute using the cell passaging tool and dislodged colonies were placed into a centrifuge tube.

4. Cells were centrifuges at 100× g for 3 min.

5. The media was aspirated and the cells resuspended in 1 mL of cell cryopreservation media (see Table of Materials).

6. Cells were divided equally into two cryovials and these were placed into a pre-chilled cell cryopreservation container at 4° C.

7. Cells were stored at −80° C. for 24-48 h.

8. Vials were transferred to either a −135° C. freezer or to a liquid nitrogen tank.

3. Human iPSC Differentiation to Macrophages

### 1. Preparing Cell Differentiation Growth Factors and Other Reagents

1. hESC-SFM media was prepared (see previous section).

2. A 0.1% w/v solution of porcine gelatin was prepared by dissolving the gelatin into sterile water. Gelatin solution was stored at 4° C. for up to 2 years.

3. Human BMP4 stock solution (25 μg/mL) was prepared by dissolving BMP4 into a 4 mM hydrogen chloride (HCl)-0.2% BSA PBS solution. The stock solution was distributed as 50 μL aliquots in cryotubes. Stock solutions were stored at −20° C. for up to 1 year. Once thawed, stock BMP4 was stored at 4° C. for up to 5 days.

4. Human VEGF stock solution (100 μg/mL) was prepared by dissolving VEGF into a 0.2% BSA PBS solution. The stock solution was distributed as 10 μL aliquots in cryotubes. Stock solutions were stored at −20° C. for up to 1 year. Once thawed, stock VEGF was stored at 4° C. for up to 7 days.

5. Human SCF stock solution (100 μg/mL) was prepared by dissolving SCF into a 0.2% BSA PBS solution. The stock solution was distributed as 5 μL aliquots in cryotubes. Stock solutions were stored at −20° C. for up to 1 year. Once thawed, stock SCF was stored at 4° C. for up to 10 days.

6. Human IL3 stock solution (10 μg/mL) was prepared by dissolving IL3 into a 0.2% BSA PBS solution. The stock solution was distributed as 500 μL aliquots in cryotubes. Stock solutions were stored at −20° C. for up to 2 years. Once thawed, stock SCF was stored at 4° C. for up to 15 days.

7. Human CSF1 stock solution (10 μg/mL) was prepared by dissolving CSF1 into a 0.2% BSA PBS solution. The stock solution was distributed as 1 mL aliquots in cryotubes. Stock solutions were stored at −20° C. for up to 2 years. Once thawed, stock SCF was stored at 4° C. for up to 15 days.

8. Separate 10 μg/mL stock solutions of interferon-gamma (IFNg), interleukin 4 (IL4), and interleukin 10 (IL10) were prepared by dissolving into 0.2% BSA PBS solutions. Lipopolysaccharide (LPS) was prepared to a stock solution of (100 U/mL) by dissolving into a 0.2% BSA PBS solution. Each stock solution was distributed as 35 μL aliquots. These were stored at −80° C. for up to 2 years. Once thawed, stocks were stored at 4° C. for up to 7 days.

### 2. Stage 1: Generation of Embryoid Bodies (EB) (Day 0-Day 3)

1. On day 0, 2.25 mL of Stage 1 media (hESC-SFM supplemented with 50 ng/ml BMP4, 50 ng/ml VEGF, and 20 ng/mL SCF) was added into two wells of an ultralow attachment 6 well plate.

2. Maintenance media of one 80% confluent well of iPSCs in a 6 well plate was replaced with 1.5 mL of Stage 1 media.

3. Colonies were cut using the cell passaging tool and cut colonies were transferred with a pipette into the two wells of an ultralow attachment 6 well plate (see Table of Materials).

4. On day 2, cytokines were brought to a final concentration of 50 ng/ml BMP4, 50 ng/ml VEGF, and 20 ng/mL SCF using 0.5 mL of hESC-SFM media.

NOTE: IPSC colonies become EBs.

### 3. Stage 2: Emergence of Hematopoietic Cells in Suspension

1. On day 4, 4 wells of a 6 well tissue culture plate were coated with 0.1% w/v gelatin and incubated for at least 10 min.

2. Gelatin was removed and 2.5 mL of Stage 2 media (X-VIVO15 supplemented with 100 ng/ml CSF1, 25 ng/ml IL-3, 2 mM glutamax, 1% penicillin-streptomycin, and 0.055 mM 2-mercaptoethanol) was added.

3. Formed EBs were collected into a 50 mL centrifuge tube and allowed to settle at the bottom of the tube by gravity. The media was aspirated carefully.

4. EBs were resuspended in 2 mL of Stage 2 media.

5. 10-15 EBs (no more than 15) were transferred to a gelatin-coated well containing 2.5 mL of Stage 2 media.

6. EBs were incubated at 37° C. and 5% CO₂ air.

7. Media on plated EBs was changed every 3-4 days for 2-3 weeks.

8. After 2-3 weeks, the EBs started releasing non-adherent hematopoietic cells into suspension. This period of suspension cell release varies and is cell line dependent. Cells in this suspension were harvested and matured into macrophages (see Stage 3).

### 4. Stage 3: Terminal Macrophage Maturation

1. Suspension hematopoietic cells were collected and media replenished (Stage 2 media) on the EB plate.

2. Suspension cells were centrifuged at 200×g for 3 min.

3. Suspension cells were resuspended in Stage 3 media (X-VIVO15 supplemented with 100 ng/ml CSF1, 2 mM glutamax, and 1% penicillin-streptomycin).

4. Collected and spun cells were plated onto untreated plastic 10 cm bacteriological-grade plates (10 mL) or uncoated 6 well tissue culture plates (3 mL) at a density of 0.2×10⁶ cells/mL.

5. Cells were kept in Stage 3 media for 9 to 11 days, changing media every 5 days. Steps 3.4.1-3.4.5 from Stage 3 could be repeated every 3-4 days and suspension cells harvested from the original EB plate for up to 3 months.

### 5. Macrophage Polarization

1. To activate macrophages to an M (LPS+IFNg) phenotype, the cells are stimulated with LPS (final concentration: 100 ng/ml) and IFNg (final concentration: 10 U/mL) for 48 h. To activate cells to an M (IL4) phenotype, cells were stimulated with IL4 (final concentration: 20 ng/ml). To activate to an M (IL10) phenotype, macrophages were stimulated with IL10 (final concentration: 5 ng/ml).

4. iPSC-Derived Macrophages Quality Control Check

2. The number of hematopoietic suspension cells produced per 6 well plate of EBs was determined by counting them with a hematocytometer.

3. Macrophage morphology was assessed as previously described (e.g., using commercial kit staining as per Lopez-Yrigoyen, M., et al. (2019), Lopez-Yrigoyen, M., et al. (2018)).

4. Detection of the expression of macrophage specific markers and polarization markers using gene expression analyses and flow cytometry as previously described (Lopez-Yrigoyen, M., et al. (2019), Lopez-Yrigoyen, M., et al. (2018)).

1. For flow cytometry experiments on one well of a 6 well plate of macrophages, cells were harvested by aspirating their maturation media, washed with 2 mL of PBS, and incubated with 2 mL of enzyme-free cell dissociation buffer for 5 min at room temperature (RT). Macrophages were detached and harvested by pipetting up and down repeatedly.

2. Cells were counted with a hematocytometer and resuspended in 80 μL of a 2% BSA, 0.5 mM ethylenediaminetetraacetic acid (EDTA)-PBS solution.

3. 20 μL of MACS human Fc blocker was added.

4. Cells were incubated on ice for 20 min and protected from light.

5. An appropriate volume of 2% BSA, 0.5 mM EDTA PBS solution was added to bring the cell concentration to 1×10⁶ macrophages/mL.

6. 1×10⁵ cells were stained in 100 μL of 2% BSA, 0.5 mM EDTA PBS solution with corresponding antibody (see NOTE below) and incubated for 15 min at RT protected from light.

7. Celled were washed 1× with at least 100 μL of 2% BSA, 0.5 mM EDTA PBS.

8. The cells were resuspended in 200 μL of 2% BSA, 0.5 mM EDTA PBS'

9. 4′,6-diamidino-2-phenylindole (DAPI, diluted 1:1,000) was added as a live-dead dye and the suspension incubated for 3 min.

5. For flow cytometry analyses, cells were gated on the main population, then single cells, and then live cells. On the live cell population, macrophage-related marker expression was evident. Antibodies were carefully titrated for each cell line used to derive macrophages. The dilution factor for SFCi55-derived macrophages flow cytometry assays is also included

### 6. High Throughput Phagocytosis Assay

1. iPSC-derived macrophages (iPSC-DMs) were harvested by aspirating the media, adding ice cold enzyme free cell dissociation buffer, and incubating for 5 min. Macrophages were collected by pipetting repeatedly.

2. 8×10⁴ iPSC-DMs were plated in a well of an imaging tissue culture grade 96 well plates (e.g., Cellcarrier Ultra, Perkin Elmer) at least 2 days before high throughput imaging in 200 μL of Stage 3 media.

3. pHrodoGreen Zymosan-A Bioparticles were prepared by resuspending one vial in 2 mL of PBS (“Solution 1”). Vortex solution for 10 s.

4. 2 mL of PBS bead suspension was diluted 1:5 with more PBS (“Solution 2”).

5. Solution 2 was sonicated for 8 s and the solution was vortexed for 10 s, then kept at 4° C. This solution was used in step 5.11.

6. The media on plated iPSC-DMs was removed and they were washed with PBS.

7. iPSC-DMs were stained with a PBS solution containing Hoechst 33342 diluted 1:20. Incubation was for 20 min at 37° C.

8. Cells were washed with PBS.

9. Cells were stained with a PBS Solution containing deep red plasma membrane stain diluted 1:1,000 (see Table of Materials). Incubation was for 30 min at 37° C.

10. Cells were washed with PBS.

11. 100 μL of bead solution kept at 4° C. was added to each well of iPSC-DMs. The plates were now ready for imaging.

12. The plate was imaged using a high content imaging system and acquiring three or more fields across the well to obtain a good representation of the well.

13. Phagocytosis was quantified using Columbus software (High-Content Imaging analysis system software). A specific algorithm was developed for unambiguous image batch analysis:

1. Blue intensity was measured and it was defined in the software that blue signal indicates the nuclei.

2. Red intensity was measured and it was defined in the software that red signal indicates the cytoplasm.

3. It was defined that nucleus and cytoplasm together corresponded to a cell.

4. Green intensity was measured in the cells and a strict cut-off/threshold value was established to consider a cell as phagocytic.

5. The phagocytic cell fraction was quantified and the average phagocytic index per cell. Bead color intensity is proportional to the number of beads, thus phagocytic activity can be measured by the number of beads ingested.

6. The algorithm/pipeline was applied to all images within every field and at all time-points acquired, allowing a robust and unbiased batch approach to determine the phagocytic capabilities of cells.

NOTE: Columbus is a high content analysis software, which offered cell segmentation analysis for cell phenotyping and functional testing.

### Derivation of Macogreen 16 GFP Line

The MacoGreen 16 GFP line was derived using a lentiviral plasmid which encodes for a 0.7 kb sequence encompassing the CBX3 gene, the EF1alpha promoter, eGFP gene, a T2A, and the Puromycin resistance gene. 24 h before infection, 4 wells of a six-well plate of early passage WT iPSC 55 iPSC cells were treated with Rock Inhibitor (Merck Cat #SCM075) at a final concentration of 3.33 ug/ml. On the day of lentiviral infection, iPSC cells were detached by washing once with PBS, adding 0.5 ml of Stempro Accutase (Gibco Cat #A110501) and incubating for 3 min. 1 ml of Stempro hESC SFM media (Thermo Fisher Cat #A1000701) was added to the well and cells were detached by pipetting gently. iPSC cells were re-suspended in Stempro hESC media, supplemented with recombinant human FGF at a concentration of 10 ug/ml (Sigma Cat #PHG0021) and 3.33 ug/ml of Rock Inhibitor. 5×10⁶ iPSC cells in single cell suspension were plated in a 10 cm dish previously coated with CTS CellStart (Invitrogen Cat #A1014201). Lentiviral particles were added to the dish to reach an MOI of 0.5. 48 h after infection, 30-40% cells were GFP positive, and treatment started with puromycin at a concentration of 0.5 ug/ml. 8 days after Puromycin selection, 48 colonies were picked and transferred individually to a well of a 24 well plate. Colonies were fed every other day and when confluent, they were moved into a well of a 6-well plate. From a 6-well plate format, 23 clones were expanded, frozen and tested for GFP expression by flow cytometry. Clone 16 looked like a single clone population of GFP+ cells. Cells from this clone expanded well and differentiated to the macrophage lineage. The number of MacoGreen 16 macrophages produced was comparable to the number of macrophages produced from the SFCi55, and they were also a single pure population of GFP expressing cells. Furthermore, MacoGreen 16 macrophages expressed human macrophage cell surface markers, including CD45, 25F9, CD163, and CD169 (expression was comparable to SFCi55 macrophages); were able to phagocytose cancer cells and had the ability of changing their phenotype after the addition of LPS+IFNg, IL4, or IL10.

### Phagocytosis Assay

GFP expressing iPSC-derived macrophages were plated at a density of 2×10⁴ per well of an imaging TC Treated 96 well plate (CellCarrier-96 Ultra Microplates, Perkin Elmer Cat #6055300) in 100 ul of macrophage maturation media: X-VIVO15 media (Lonza, Cat #BE02-060F) supplemented with 100 ng/ml recombinant human CSF1 (Biolegend Cat #574808), 2 mM Glutamax (Invitrogen Cat #35050038) and 1% Penicillin-Streptomycin (Gibco Cat #15140-122).

On the day of the assay, 24 hr-48 hr post macrophage seeding, Jurkat WT cells or Jurkats-HLA-G over-expressing cells were stained with IncuCyte® pHrodo® Orange Cell Labelling Kit for Phagocytosis (Sartorius Cat #4766). Briefly, cells were collected, centrifuged for 4 minutes at 200 g, and washed with IncuCyte pHrodo Cell Wash Buffer (1×10⁶ cells/ml of wash buffer). Cells were spun down for 4 minutes at 200 g and pellet was then resuspended in IncuCyte pHrodo Cell Labelling Buffer to a density of 1×10⁶ cells/ml. Solubilized IncuCyte pHrodo Orange Cell Labelling Dye was added to the cell suspension at a final concentration of 600 ng/ml. Cell suspension was mixed and then incubated 1 hr at 37° C. (cells were mixed every 20 min of incubation time). To remove excess IncuCyte pHrodo Labeling Dye, cells were centrifuged at 1300 rpm for 7 minutes. Cell pellet was then re-suspended at a density of 1.2×10⁶ cells/ml in macrophage maintenance media. 50 ul of cell suspension (60,000 cells) were placed into each macrophage containing well (macrophage: target cell ratio is 1:3). Test antibodies or IgG4 Isotype control were added at a final concentration of 10 ug/ml in technical triplicates. Plate was immediately placed into the Incucyte S3 Live imaging system. Acquisition of 4 fields per well was carried out every 30 min for 7 hrs. Image Analyses were carried out using the Cell by Cell pipeline from Incucyte. Briefly, macrophages were segmented based on GFP expression and size. Total orange fluorescence in the macrophage population was the first output taken post cell by cell analysis. By setting an orange intensity threshold, the phagocytic macrophage fraction was determined, as well as the average orange intensity in the phagocytic macrophage population.

### Reprogramming Assay

### Cell Plating and Treatments

Macrophages were harvested by aspirating the media and adding 1.5 ml of Enzyme free cell dissociation buffer (Life technologies, Cat. 13151014) to each well (6 well plate). Cells were incubated for 4 minutes at room temperature, and pipetted vigorously to detach. Cell suspension was collected and centrifuged at 200 g for 3 minutes. Supernatant was aspirated and cells resuspended in Macrophage maturation media (X-VIVO15 (Lonza, Cat. BE02-060F) supplemented with 100 ng/ml CSF1 (BioLegend, Cat. 574808), 2 mM glutamax (Life Technologies, Cat. 35050038), and 1% penicillin/streptomycin (Life technologies, Cat. 15140122). Cells were plated onto uncoated 24-well tissue culture plates at a density of 1.25×10⁵ cells per well in a final volume of 375 ul per well. The macrophages were incubated overnight at 37° C. and 5% CO₂. Next morning, cell medium was refreshed and 10 ug/ml of each antibody/control was added in a final volume of 375 ul and incubated at 37° C. and 5% CO₂ for 1 hour. Subsequently, LPS diluted in Macrophage maturation media was added at a final concentration 1 ng/ml or 25 ng/ml and placed back into the incubator for 5 hours. After the incubation period, macrophage supernatant was collected and transferred to labelled 1.5 ml Eppendorf tubes, spun in a microcentrifuge at 200×g for 3 minutes and 350 ul of supernatant was carefully transferred to new, labelled Eppendorf tubes.

### ELISA for Cytokines Quantification

ELISAs were performed according to manufacturer's instructions. GM-CSF: Human GM-CSF DuoSet ELISA (R&D Systems. Cat. DY215-05). Undiluted supernatants are used for GM-CSF ELISA. TNFα: human TNFa ELISA MAX Deluxe Set (BioLegend. Cat. #430204). The following supernatant dilutions are used for TNFα ELISA: 1:200 for macrophages treated with 25 ng/ml of LPS, 1:100 for macrophages treated with 1 ng/ml of LPS and 1:50 for non-LPS treated macrophages.

### ELISA Results Calculation

The analysis of the results is performed using GraphPad Prism or custom R scripts using a four parameter logistic (4-PL) regression.

### Results.

### Heterogeneous Expression of MHC Class I Binders LILRB1, LILRB2, and LILRA3.

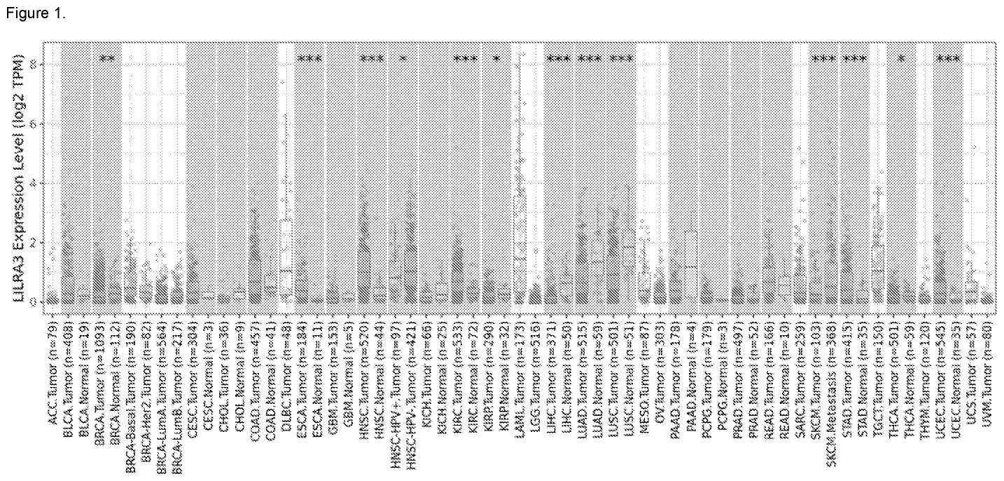

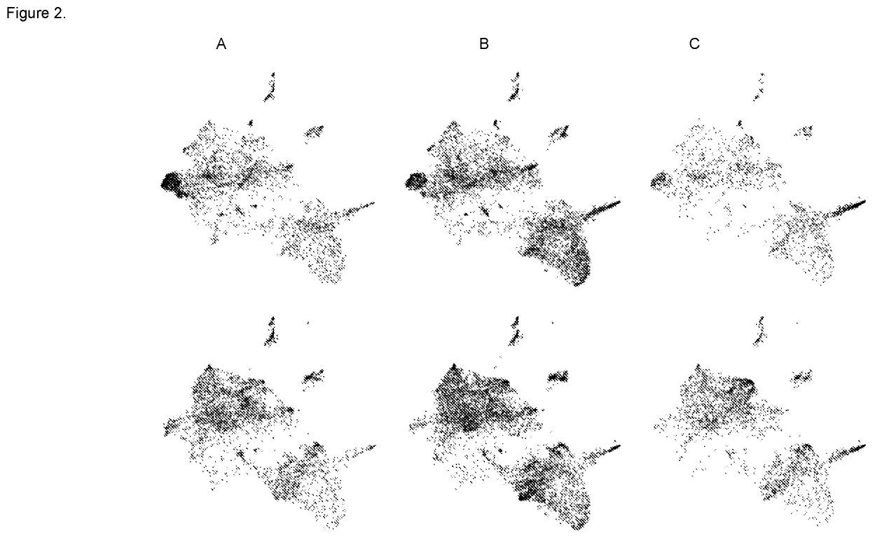

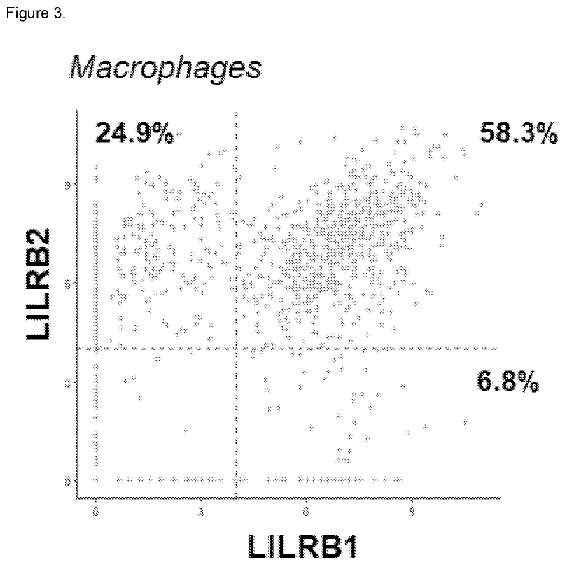

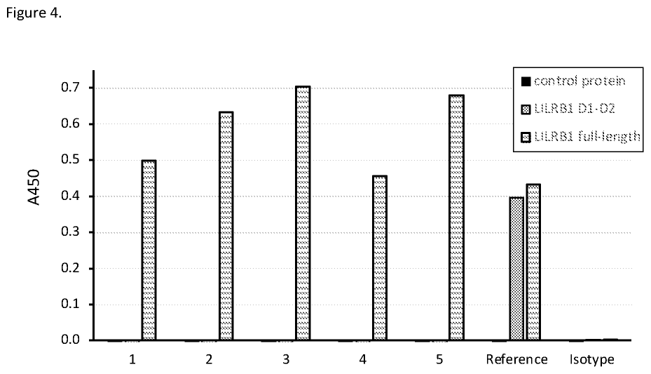

Members of the LILRB and LILRA families have both distinct and overlapping tissue expression patterns, ligands, and biological functions. For example, both LILRB1 and LILRB2 can bind HLA-G and HLA-A, whereas LILRB2 expression is restricted to the myeloid compartment and LILRB1 is not. Moreover, some family members have intracellular ITIM domains (for example LILRB1, LILRB2 and LILRB3) and some ITAM domains (for example, LILRA1). This raises the possibility of both redundant and non-redundant biology. Of particular interest are the MHC class I binders, a family of molecules expressed on tumours that are associated with immune regulation. To explore this further bioinformatic analysis of a single cell RNA sequencing dataset (Mulder et al. 2021) was performed, showing that LILRB1 and LILRB2 were both up-regulated in TAMs from multiple cancer types, but that expression was mixed with LILRB1, LILRB2, or LILRB2 and LILRB1 dual positive TAMs detected FIG. 2. As expected, LILRB1 was also expressed on NK cells, B-cells, and T-cells. Further bioinformatic analysis of RNA sequencing data from TAMs in melanoma cancer patients (Mulder et al. 2021) showed that in 58% of TAMs both LILRB1 and LILRB2 were detected, in 7% of TAMs LILRB1 alone was detected, and in 25% of TAMs LILRB2 alone was detected FIG. 3. Since LILRB1 and LILRB2 are both immunosuppressive this suggests that all three populations must be targeted to maximise therapeutic activity. Separately, to explore the role of LILRA3 in cancer biology, bioinformatics analysis comparing of LILRA3 RNA expression between multiple normal and cancer tissues was undertaken using the TCGA. This showed that LILRA3 was over-expressed (p<0.05) in head and neck squamous sarcoma, oesophageal carcinoma, renal cell carcinoma, stomach adenocarcinoma, thymus cancer, and endometrial carcinoma FIG. 1.

Therapeutic approaches targeting both LILRB1 and LILRB2 will have the advantage of increasing the percentage of macrophages activated in a tumour microenvironment where expression of these molecules is heterogeneous, and of reducing potential redundancy between molecules with high homology, overlapping expression and ligand binding. Human genetic data suggesting LILRA3 is anti-inflammatory, and increased expression of LILRA3 in tumours, suggests that targeting LILRA3 may also be advantageous.

### Example—MHCI Deregulation in Cancer and LILRB1/2 Antibodies in the Art

The ligands for LILRB1, LILRB2, and LILRA3 include classical and non-classical MHC class I molecules. HLA-G is an example of a non-classical MHC class I and HLA-A, B, C are examples of classical MHC class I molecules. Classical MHC class I down-regulation is a known tumour immune evasion mechanism, reducing tumour antigen presentation and T cell activation (https://pubmed.ncib.nlm.nih.gov/32630675/). For example, down-regulation of classical MHC class I is found in approximately 1 in 3 melanoma patients and is associated with TGFbeta signalling and innate and acquired resistance to T cell checkpoint therapies. In contrast, up-regulation of non-classical MHC class I, such as HLA-G, is also a known tumour immune evasion mechanism.

Antibodies that bind LILRB1, LILRB2 or LILRB1 and LILRB2 are described in the art (e.g. as described in the background to the invention and shown in Tables 1 to 4). However, these antibodies block the ligand interaction of LILRB1 and/or LILRB2, and blockade was used to identify antibodies with desirable properties. Furthermore, it is also reported that non-blocking antibodies do not have activity in functional macrophage assays. Ligand blocking has been determined through preventing interaction of ligand with receptor. “By “no blocking activity” or “non-blocking” or “not blocking”, it is meant that in an assay described herein the assay signal is more than 10% of the signal observed for the isotype control. The Isotype control is 100% of signal, blocking is less than 10% of the signal observed for the isotype control, non-blocking is more than 10% of the signal observed for the isotype control. Hitherto, there has been no description of a human antibody that binds human LILRB1, human LILRB2 and human LILRA3 and that does not block interaction of LILRB1 or LILRB2 with their respective ligands (e.g., LA-G/A/E).

### Example—LILRB1 Non-Blocking Antibodies Identified

### Results

Surprisingly, of the five most active antibodies in our assay cascade that bind human LILRB1, human LILRB2 and human LILRA3, all were non-ligand blockers, despite representation of ligand blocking and non-ligand blocking antibodies in modes of action in our larger antibody panel.

### Identification of LILRB1 Non-Ligand Blocking Antibodies.

LILRB1 binds to its ligands, classical and non-classical HLA molecules, and by that can transmit the immunosuppressive signal to tumour associated macrophages, and other immune cells. This signalling occurs via the ITIM domains present in various immune-regulatory receptors expressed on immune cells, such as Fc receptors, PD-1, TIGIT, and PECAM-1. Although, generally considered an inhibitory domain, ITIM domains can also transmit activatory signals under some circumstances (Coxon et al. Blood (2017) 129 (26): 3407-3418). Antibodies whose activity can positively modulate immune cells in the immune-suppressive tumour micro-environment independent of the target receptor-ligand interactions are preferred. Furthermore, as LILRB1 is widely expressed on TAMs in multiple types of cancer, of which only a subset will be engaged in the direct interaction with the cells expressing their targets, the antibodies which could exert biological activity in target cells without inhibiting the ligand induced signalling are preferred.

Binding of HLAs to LILRB1 occurs via the domain 1 (D1) and domain 2 (D2), amino acids 24-224 of human LILRB1 ectodomain (SEQ ID NO: 41).

Antibodies obtained by immunization of humanized mice with the human LILRB1 protein were tested in an ELISA for their ability to bind to the full-length LILRB1 ectodomain (SEQ ID NO: 41) and to the truncated version of the human LILRB1 protein containing domain 1 and 2 (SEQ ID NO: 42). Five antibodies (Antibody 1, 2, 3, 4 and 5) were selected that were capable of binding to a full length LILRB1 ectodomain of LILRB1 (SEQ ID NO: 41), but were not able to bind to the truncated (D1-D2) version of the LILRB1 ecto-domain (SEQ ID NO: 42). The 5 selected clones, unlike the reference Ab (Reference Antibody 1), bind only to the full length LILRB1 protein, and not to its truncated form (FIG. 4).

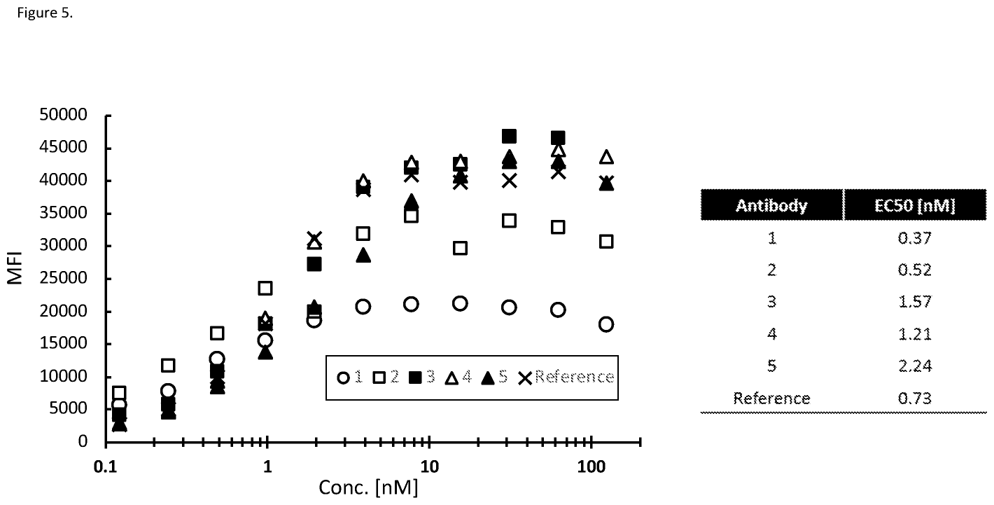

The ability of selected anti-LILRB-1 antibodies to bind to the LILRB1 receptor in its native context of the cell membrane was investigated by flow cytometry. HEK293 cells over-expressing full length human LILRB1 (SEQ ID NO: 45) were incubated with the increasing amounts of the antibodies and the cell bound antibodies were detected using PE-labelled secondary antibodies using a flow-cytometer. As shown in (FIG. 5), all 5 clones showed strong binding to the cell membrane embedded LILRB1 with calculated EC50 ranging from 0.37 nM to 2.24 nM; the reference antibody (Reference Antibody 1) showed intermediate EC50 of 0.73 nM. “LILRB1 binding” denotes that in this assay EC50 was lower than 100 nM.

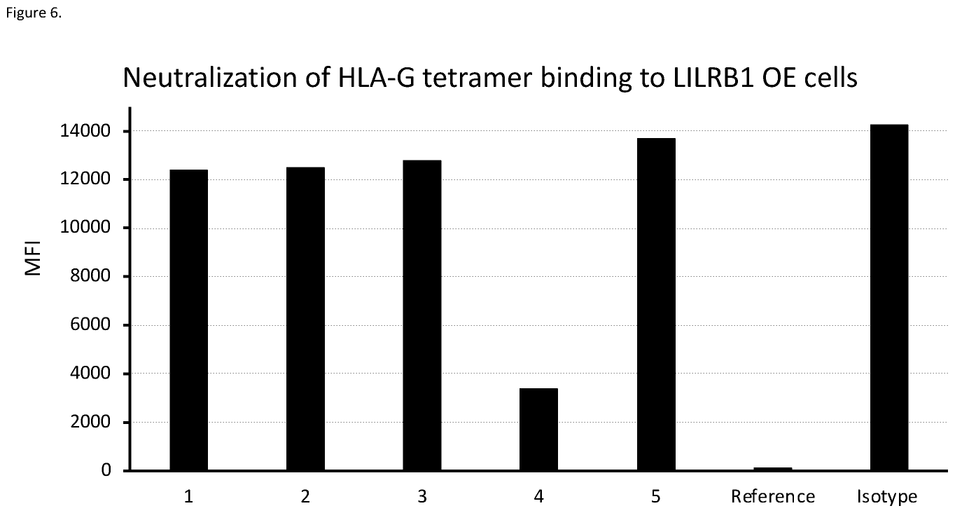

HLA-G is a main ligand of LILRB1 found to be over-expressed in various tumours. We therefor tested the ability of the selected anti-LILRB1 Abs to block binding of the receptor to its ligand. For that, HEK293 cells over-expressing human LILIRB1 receptor were incubated with human HLA-G PE-labelled tetramers, in the absence or presence of 500 nM test antibodies. Cell bound HLA-G was quantified by flow cytometry. As shown in FIG. 6, Antibody 1, 2, 3, 4, and 5 showed no blocking activity at this high concentration. In contrast, the presence of the reference antibody (Reference Antibody 1) blocked binding of the HLA-G to the cells. “No blocking activity” denotes that in this assay signal was more than 10% of the signal observed for the isotype control.

### Binding to LILR Family Members.

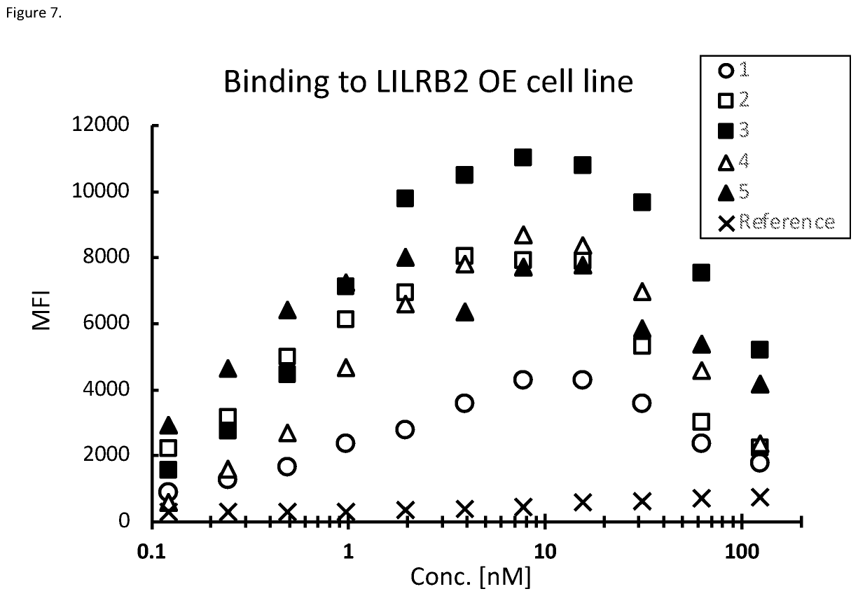

The ability of the selected antibodies to bind human LILRB2 was investigated by flow cytometry. HEK293 cells over-expressing full length human LILRB2 receptor (SEQ ID NO: 46) were incubated with the increasing amounts of the anti-LILRB1 clones, and the bound antibodies detected by flow cytometry as described below in “Identification of LILRB1 non-neutralizing antibodies”. Unlike the Reference Antibody 1, all 5 selected antibodies of the invention bind LILRB2 with low nM affinities (FIG. 7).

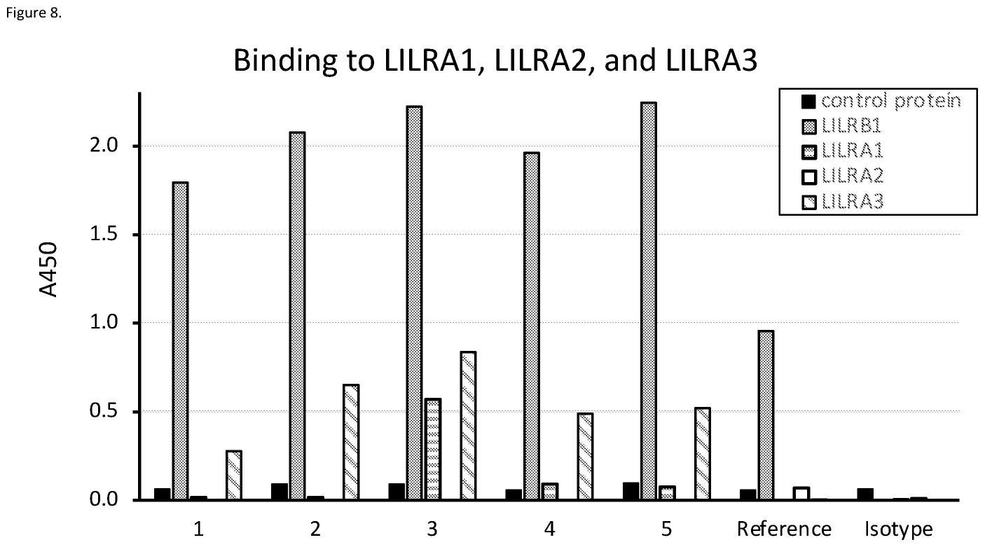

Binding to the most similar LILRA family members was tested by ELISA using recombinant, full-length ectodomains of LILRA1 (SEQ ID NO: 47), LILRA2 (SEQ ID NO: 48), LILRA3 (SEQ ID NO: 48). As shown in FIG. 8, unlike the Reference Antibody 1, Antibodies 1-5 bind to LILRA3. In this preliminary experiment only Antibody 3 was observed to bind to LILRA1, however subsequent ELISA demonstrated that Antibodies 1-5 bind LILRA1. None of antibodies 1 to 5 bind to LILRA2. In this context, “binding” denotes that in this assay a signal obtained for LILR protein was at least more than 3 fold higher than that observed for the control protein. To better understand the binding of Clones 1-5 to individual family members, multi point ELISA was performed and EC₅₀s for the binding were determined.

### Binding to LILRB1 Allelic Forms.

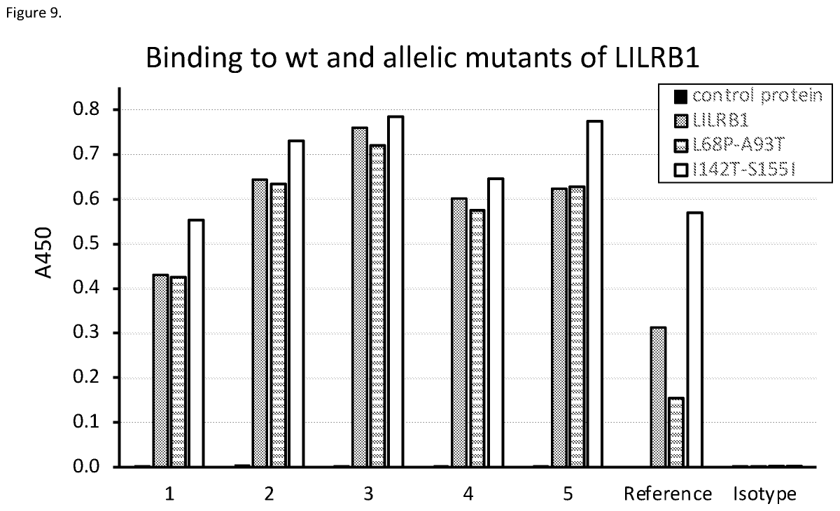

There are four major allelic variants within the ecto-domain of LILRB1 in human population [Human Molecular Genetics, 2005, Vol. 14, 2469-2480]; namely c.203T>C (L68P), c.277G>A (A93T), c.425T>C (I142T), and c.464G>T (S155I). Although they are localized in domain 1 and 2, they still could affect the overall tertiary structure of the proteins, and by that reduce the target population for the therapeutic. For that, the antibodies which bind all major allelic forms equally well are preferred. To investigate the ability of the selected anti-LILRB1 antibodies to bind the allelic forms of human LILRB1. Recombinant protein variants of human LIRB1 ectodomain, with the amino acid substitutions corresponding to the combined first two allelic forms (SEQ ID NO: 50), or to the combined third and fourth allelic form (SEQ ID NO: 51) fused to mouse IgG2a Fc were expressed in mammalian cells and purified by Protein A chromatography. These proteins were then used in ELISA. As shown in FIG. 9, unlike the Reference Antibody 1, the binding of Antibodies 1 to 5 to the allelic variants of human LILRB1 was similar to that of wild-type “wt” protein. In this context, “binding” denotes that in this assay a signal obtained for LILRB1 variant was at least more than 3 fold higher than that observed for the control protein.

### Binding to Non-Human Primate LILRB Proteins.

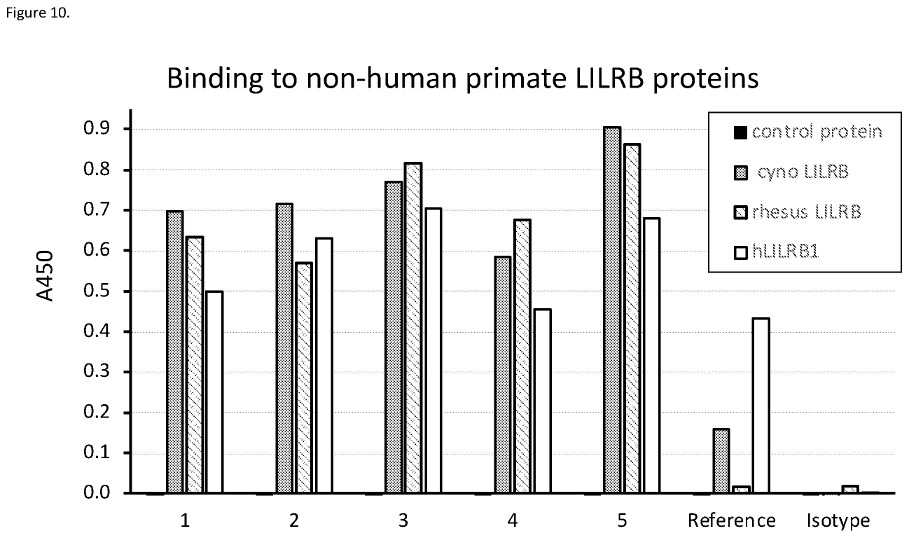

There is a single LILRB1 homolog in rhesus (Macaca mulatta) monkey (SEQ ID NO: 52) and in cynomolgus (Macaca fascicularis) (SEQ ID NO: 53), respectively. Antibodies cross-reactive to non-human primate homolog proteins are preferred as they could be used in these species to investigate the pharmacological properties of the antibodies. Recombinant rhesus and cynomolgus LILRB1 homologs were produced as fusions to mouse IgG2a (SEQ ID NO: 43 and SEQ ID NO: 44, respectively) and these proteins were used in ELISA. As shown in FIG. 10, unlike Reference Antibody 1, Antibodies 1 to 5 bind rhesus and cynomolgus LILRB proteins and human LILRB1 equally well. In this context, “binding” denotes that in this assay a signal obtained for LILRB protein was at least more than 3 fold higher than that observed for the control protein.

### Human PBMC Cytokine Release Assay

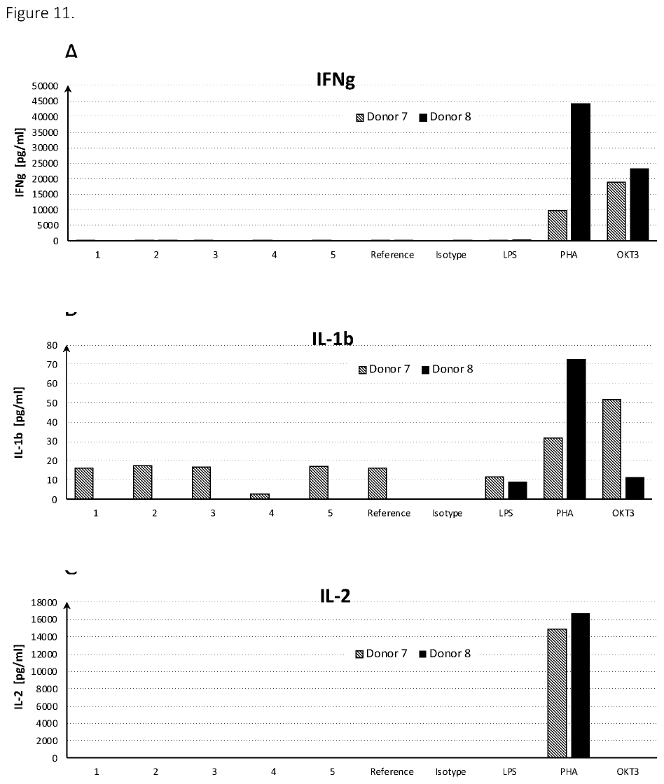

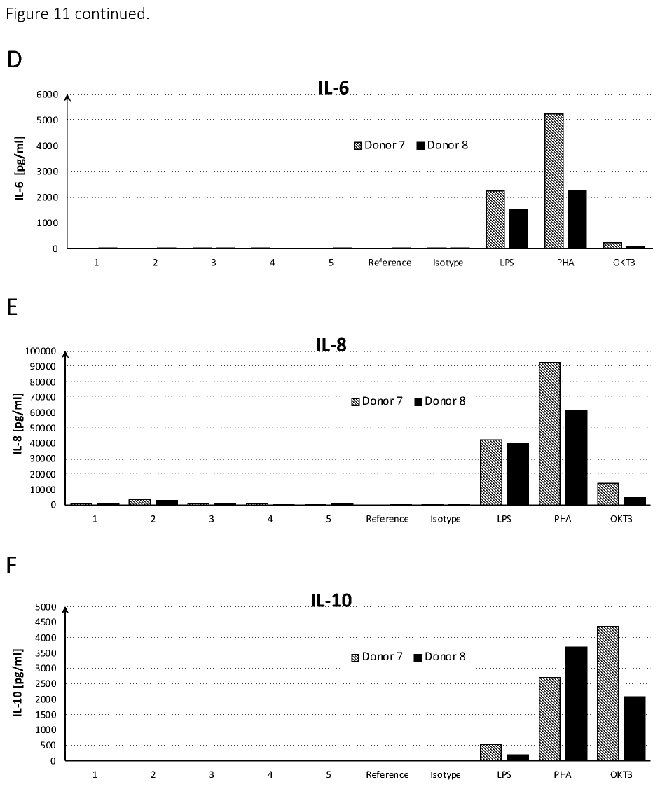

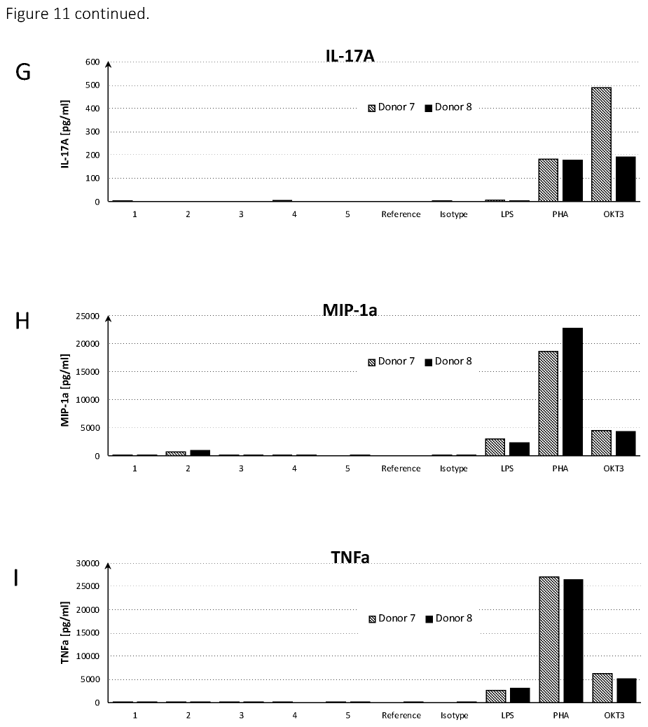

In rare cases, injection of therapeutic antibodies to human can result in the uncontrolled production of cytokines from the immune cells in blood, so called cytokine-release syndrome (CRS). Thus, antibodies that do not cause the spurious activation of immune cells resulting in the cytokine release are preferred. Clones 1-5 were tested in the standard cytokine-release storm assay in which PBMCs from two donors were incubated with 20 ug/ml of test antibodies for 24 h. After that, various cytokines were measured in the media from the treated cells. As shown in FIG. 11, none of Clones 1-5 induced the release of significant amounts cytokines, indicating they would be safe to use in human.

Antibodies 1 to 5 Bind iPS-Derived Macrophages and NK92 Cells

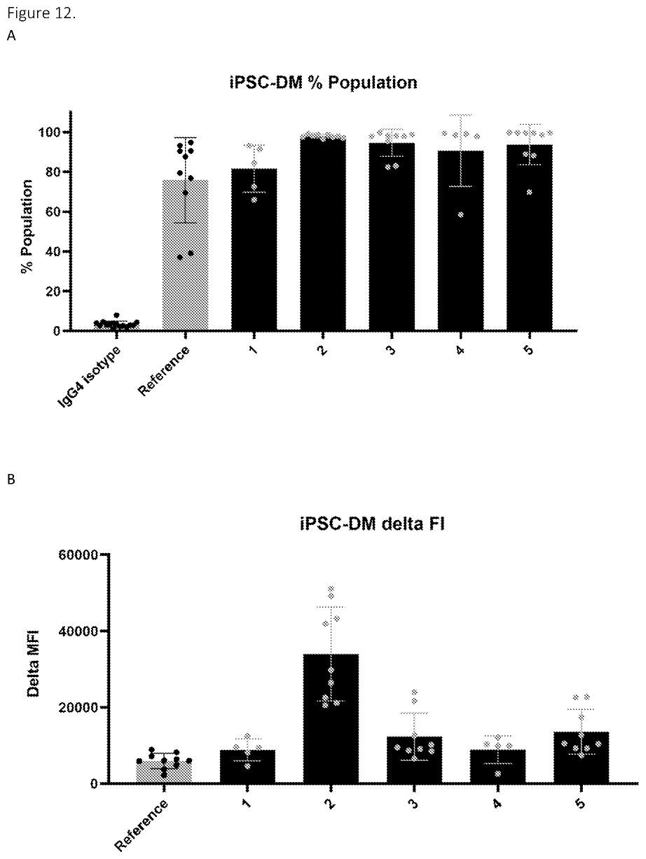

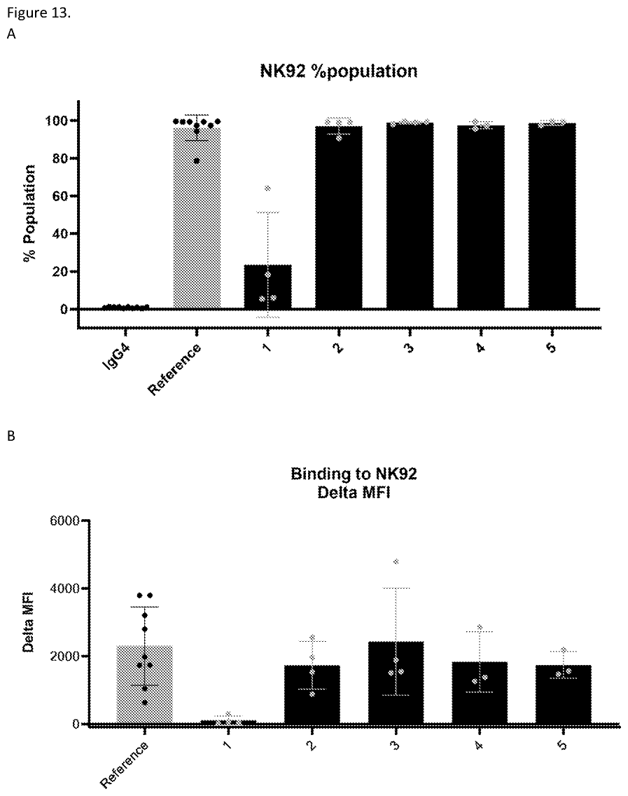

Antibodies 1 to 5 were able to bind iPS-derived macrophages (FIG. 12) and Antibodies 2 to 5 were able to bind NK92 cells (FIG. 13).

### LILRB1 Non-Blocking Antibodies Enhance Cancer Cell Phagocytosis in Macrophages.

Antibodies that were ligand blockers and also those that were ligand non-blockers were progressed through a functional assay cascade to determine the biological performance of the antibodies in therapeutically-relevant assays, namely macrophage phagocytosis and reprogramming.

We performed phagocytosis assays using MHCI deficient cell lines (DLD1), MHCI (HLA-ABC) positive (JURKAT) and HLAG overexpressing lines (JURKAT HLAG).

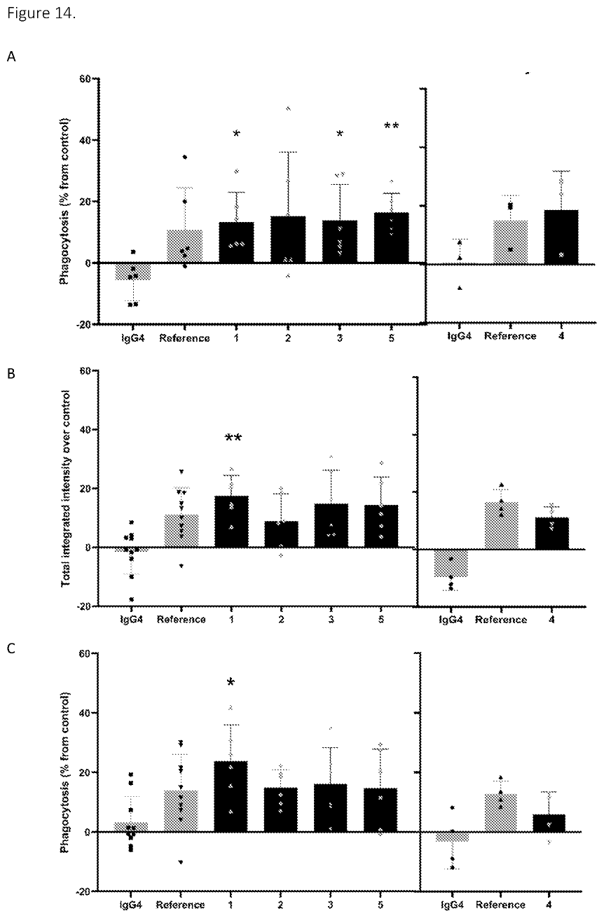

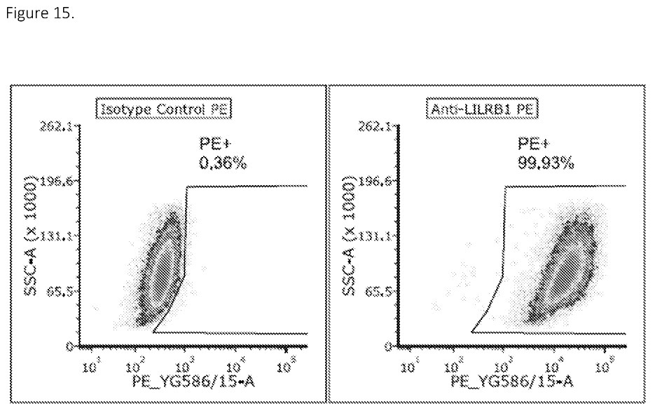

Surprisingly we observed that Antibodies 1 to 5 were superior to Reference Antibody 1, an anti-LILRB1 ligand blocking antibody, for inducing higher levels of cancer cell phagocytosis. As can be seen in FIG. 14 the ligand blocking Reference Antibody 1 did not induce significant increases in DLD colorectal cancer cell phagocytosis by macrophages, a cell line that is negative for MHC class I expression whereas 3 of the 5 of our non-ligand blocking antibodies (Antibody 1, 3 and 5) were able to induce phagocytosis in the absence of MHC Class I ligands.

Changes in MHC class I ligand expression are common in cancer, including in immunotherapy resistant disease where high unmet need remains; the non-ligand blocking antibodies of the invention are not limited by MHC-1 expression in cancer and may provide new treatment options for patients.

Our antibodies were also able to induce phagocytosis in a superior (Antibody 1) or comparable (Antibody 2 to 5) way to the Reference Antibody 1 in MHCI positive cells (FIG. 14).

Of note, antibodies produced by clones 1 to 5 were able to enhance high levels of phagocytosis in macrophages without the need for any additional co-treatment, such as anti-CD47 antibodies or an anti-EGFR antibody, which has been reported as being necessary by others (Barkal et al. ibid.; WO2021222544 (NGM)).

### Antibodies 1 to 5 Enhance Macrophage Reprogramming

Some studies reported that blocking LILRB2 on macrophages reprograms macrophages to an anti-tumoural phenotype; LILRB2 antagonism rendered macrophages resistant to humoral cytokine-dependent STAT6 activation by IL-4, relieved the suppressive effect of macrophages on T cell proliferation, and reprogrammed human macrophages from A549 lung tumour models and primary human non-small cell lung carcinoma. Furthermore, LILRB2 blockade changed the tumour microenvironment and promoted anti-tumour immunity when used in conjunction with anti-PD-L1 (Chen et al JCI 2018).

LILRB1 blockade has been linked to increased phagocytosis on macrophages (Barkal et al.) and NK increased cytotoxicity (Chen et al, JITC, 2020), and changes in macrophage activation markers following differentiation from monocytes in the presence of LILRB1 antibody.

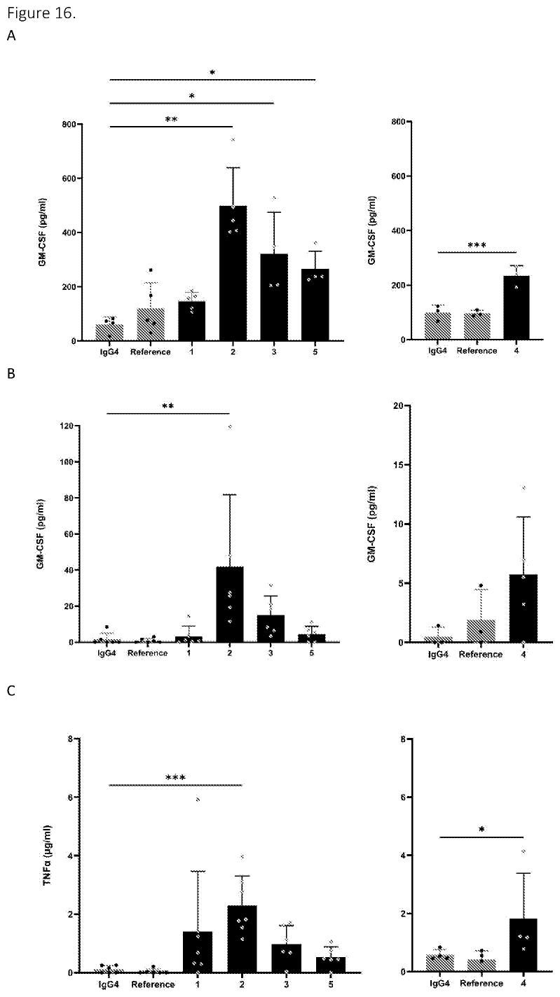

As shown in FIG. 16, Antibodies 1 to 5, but not the anti-LILBR1 Reference Antibody 1, were able to strongly induce GM-CSF release (a marker of a macrophage reprogramming) in macrophages upon LPS stimulation; among all the antibodies tested, Antibody 2 enhanced GM-CSF production by almost 5-fold compared to IgG4 isotype control.

Antibodies 2 and 4 were also able to induce significantly higher levels of the pro-inflammatory cytokine TNFa in iPS-derived macrophages upon LPS stimulation (FIG. 16).

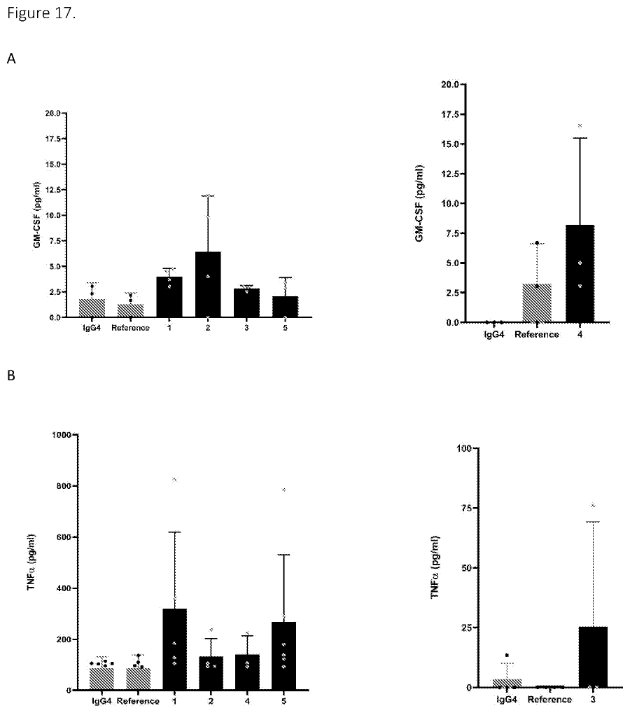

No reprogramming effects were observed when macrophages were incubated with Antibodies 1 to 5 in the absence of LPS stimulation (FIG. 17).

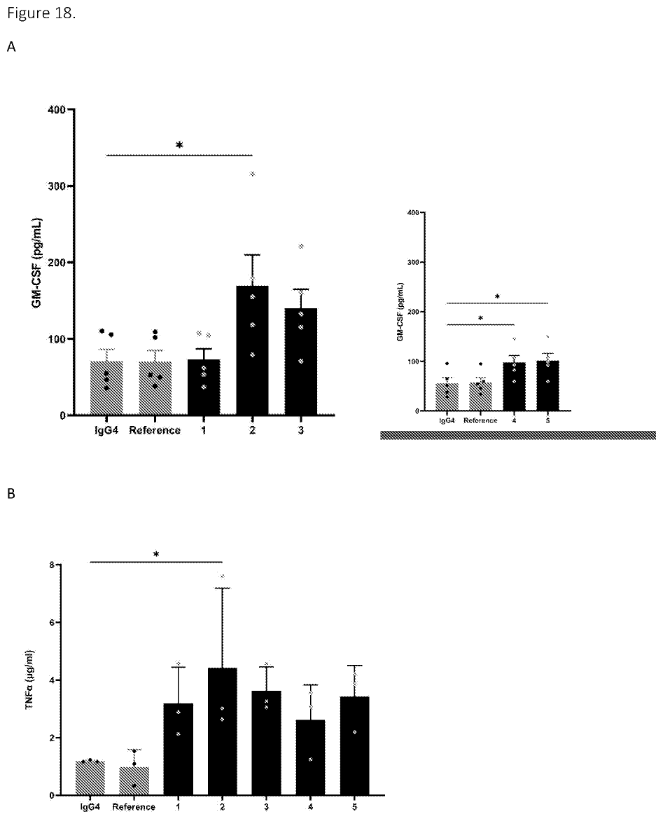

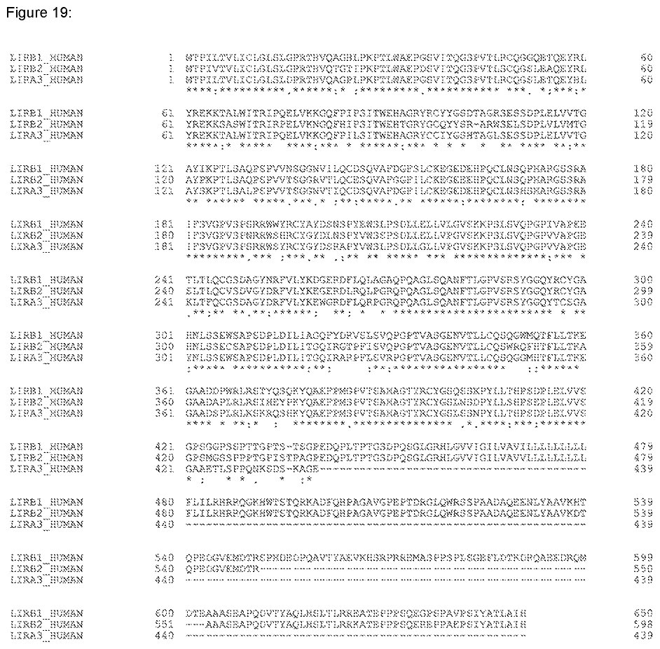

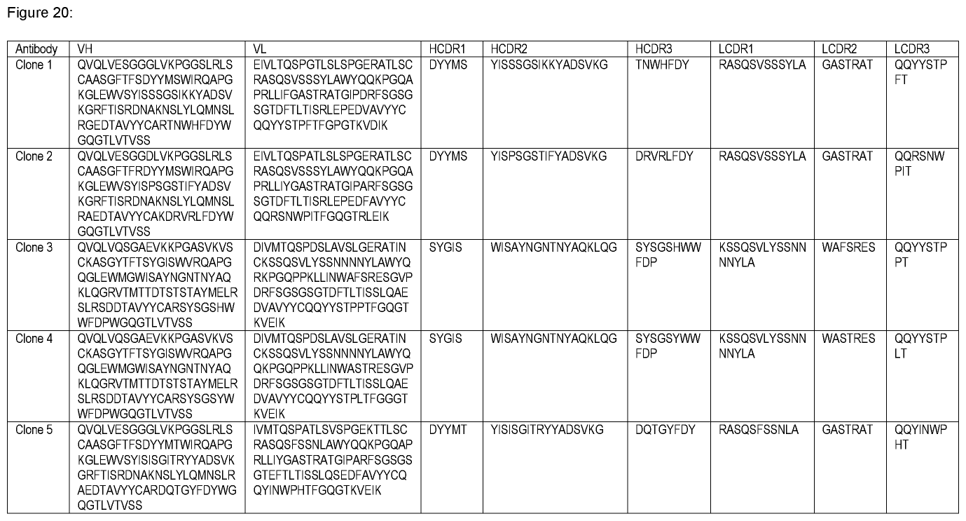

The reprograming effects were also confirmed using primary monocyte derived macrophages as shown in FIG. 18.

The non-blocking Antibodies 1 to 5 were able to induce phagocytosis of MHCI negative and positive cancer cell lines and to induce reprogramming of macrophages.

### Discussion

Antibodies 1 to 5 were identified, each of which binds specifically to LILRB1, LILRB2, LILRB3, LILRA1, LILRA3, LILRA4 and LILRA6 on human macrophages. LILRB1 and LILRB2 are key signaling receptors employed by tumour-associated macrophages to mediate immune suppression in solid tumours. LILRB1 and LILRB2 share ligands and both receptors transmit inhibitory signals to immune cells via their ITIM motifs. In addition, the inhibitory function of LILRB3 on monocytes and macrophages was recently demonstrated (Yeboah et al. JCI 2020). Hence, the approach to target three inhibitory receptors simultaneously may offer the advantage of avoiding redundant escape mechanisms in the tumour microenvironment. Moreover, a dual binding antibody provides broader coverage of the TAM population, as TAMs differ in LILRB1 and LILRB2 expression patterns: our analysis (FIG. 2) of several solid tumour types demonstrates that albeit the majority of TAMs co express LILRB1 and LILRB2, there are TAMs detectable which express only LILRB1 or LILRB2. Therefore, the dual LILRB1/2 binding properties of Antibodies 1 to 5 allow full coverage of single LILRB1-, single LILRB2- or dual LILRB1- and LILRB2-expressing immune cells.

LILRA3 is a soluble factor with anti-inflammatory activity. Unlike LILRB1 and LILRB2, LILRA3 is biologically active in its soluble form. The antibodies in this invention bind to LILRA3 and may derive benefit from targeting additional receptors in the community of LILR family members.

Prior art attributes specific mechanisms required to achieve anti-tumoural phagocytosis activity and macrophage reprogramming. In particular, LILRB1 inhibition of its interaction with MHCI, mainly HLA-G and ß2M is associated with increased phagocytotic activity by macrophages. To achieve such activity, it has been postulated that blocking of the formation of the HLAG-ß2M-LILRB1 complex is required.

Here we report a novel mechanism of increased phagocytic activity by non-ligand-blocking anti-LILRB1 antibodies. These antibodies increase phagocytic activity in the absence of MHCI, as demonstrated using the MHCI-deficient DLD1 cell line.

### CONCLUSION

In conclusion we report a novel class of human LILRB1/LILRB2/LILRB3/LILRA1/LILRA3/LILRA4/LILRA6 targeting antibodies which show strong reprogramming activities without blocking ligand-receptor interaction.

### Neutralization of HLAs Binding to LILRB1 and LILRB2

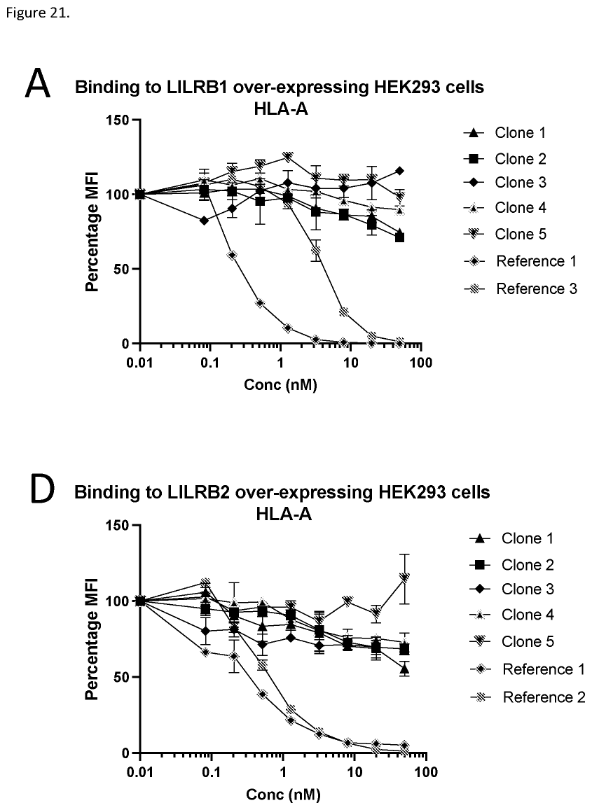

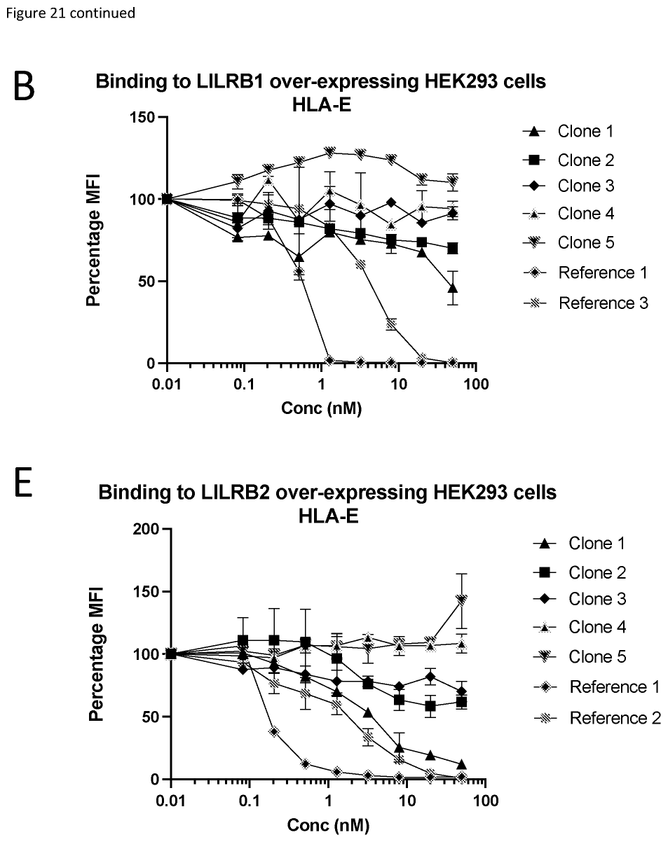

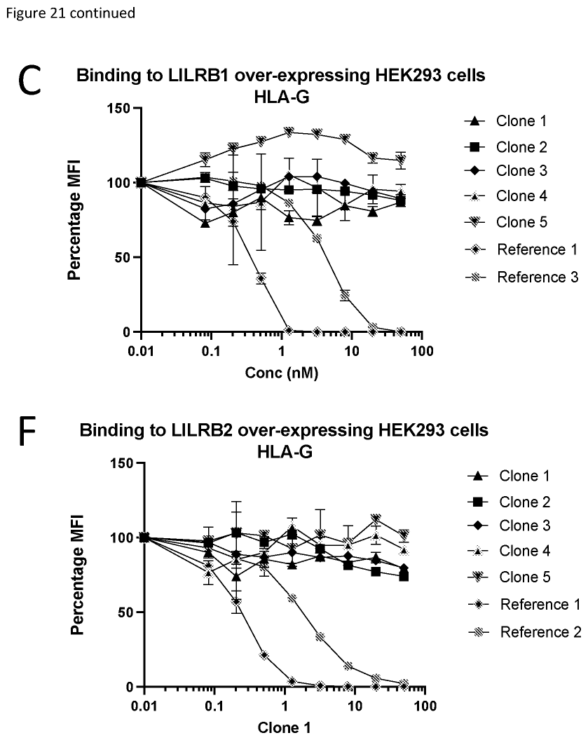

As demonstrated above, even at high concentrations, unlike the reference antibodies, Antibodies 1 to 5 did not efficiently neutralize binding of HLA-G to LILRB1. To investigate if the Antibodies 1 to 5 could neutralize binding of other HLA-As to LILRB1 or to LILRB2, HEK293 cells overexpressing the receptors were incubated with constant concentrations of PE-labelled HLA-A2, HLA-E, and HLA-G multimers and increasing concentrations of Antibodies 1 to 5 or the reference antibodies. The ability to neutralize binding of HLA to the cells was determined by flow cytometry (FIG. 21). None of Antibodies 1 to 5 neutralized binding of HLA-A (A), HLA-E (B), and HLA-E (C) to LILRB1 protein, up to the concentrations of 100 nM, whereas Reference 1 antibody (LILRB1 specific) and Reference 3 antibody (LILRB1/2 specific) neutralized the binding with an IC₅₀ in the sub- or low-nM range. Similarly, none of Antibodies 1 to 5 neutralised efficiently the binding of HLA-A (E) and HLA-G (G). Antibody 1 neutralised binding of HLA-E (F) to LILRB2, but antibody clones 2 to 5 did not neutralise binding of HLA-E (F) to LILRB2. Reference Antibody 2 (LILRB2 specific) and Reference Antibody 3 (LILRB1/2 specific), inhibited the binding for all HLAs, with IC₅₀ with sub- or low-nM range.

### Reprogramming of Immunosuppressed MDMs

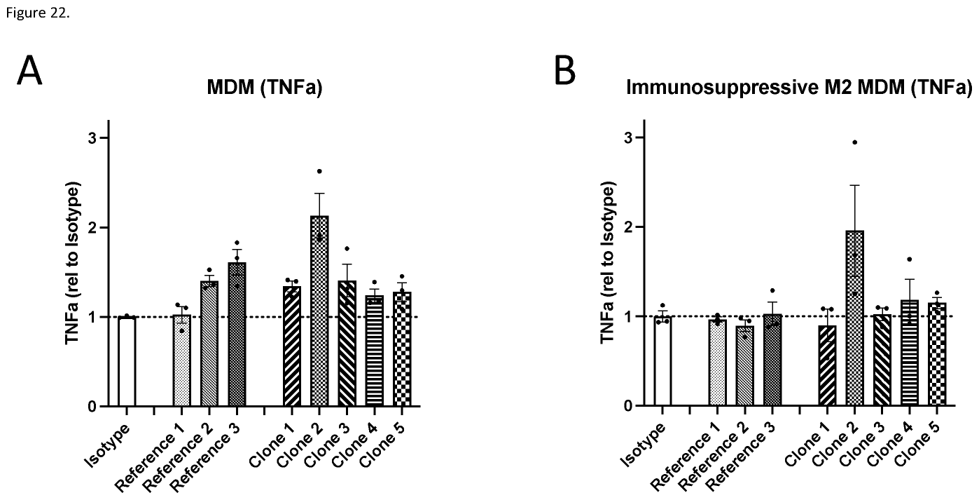

Human normal (M0) or immunosuppressed (M2) (grown in the presence of 50 ng/ml IL-10 and 50 ng/ml TGFβ) monocyte-derived macrophages were incubated with 10 ug/ml of Antibodies 1 to 5, or of isotype control, or of Reference Antibodies 1 to 3 for 1h, and then stimulated with 1 ng/ml or 10 ng/ml, for M0 and M2 conditions respectively, of LPS for 5h. Levels of TNFα were measured by ELISA (FIG. 22). As shown before, normal MDMs in the presence of CSF-1 (A) could be reprogrammed with Reference Antibody 2 (LILRB2 specific Ab), with Reference Antibody 3 (dual LILRB1/LILRB2 specific Ab), and with Antibodies 1-5, but not with isotype control or Reference Antibody 1 (LILRB1 specific Ab). In contrast, in presence of CSF-1 and IL-10 and TGFβ, two of the major immunosuppressive cytokines (B), none of the reference Ab was able to induce additional secretion of TNFα, whereas Clone 2, and Clones 4-5, yielded increased TNFα levels. This indicates that these clones could reprogram highly immunosuppressed macrophages, which are a model for TAMs present in the tumour micro-environment.

### Immunogenicity

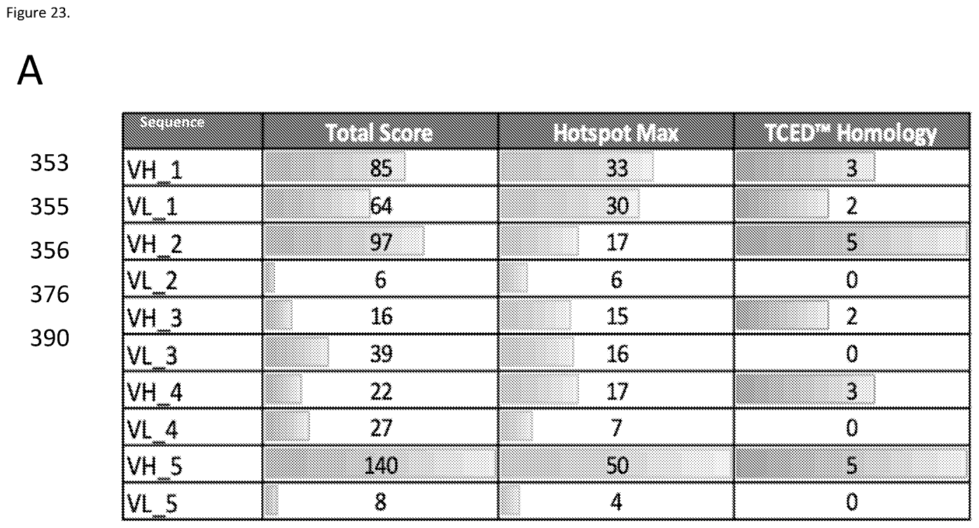

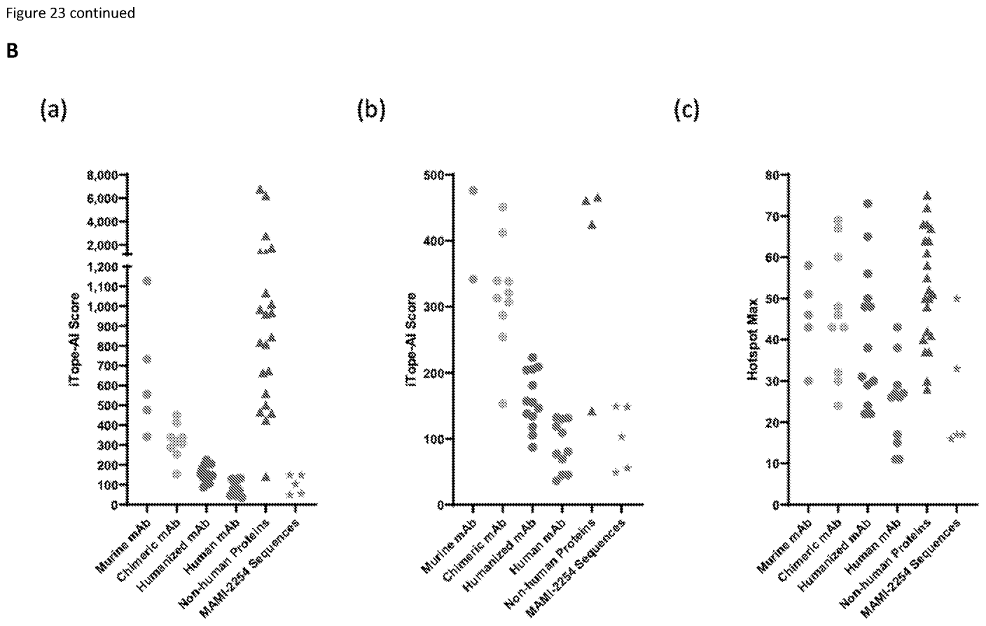

Immunogenicity of VH and VL sequences of Antibodies 1 to 5 was assessed by a CRO; Abzena; using their proprietary in silico technologies: the iTope-AI and TCED™ (FIG. 23). The two methods estimated Antibodies 1 to 5 to have an average risk of inducing immunogenicity in humans, comparable to other fully human therapeutic Abs, and lower than mouse, chimeric, or humanized therapeutic antibodies.

### Epitope Mapping

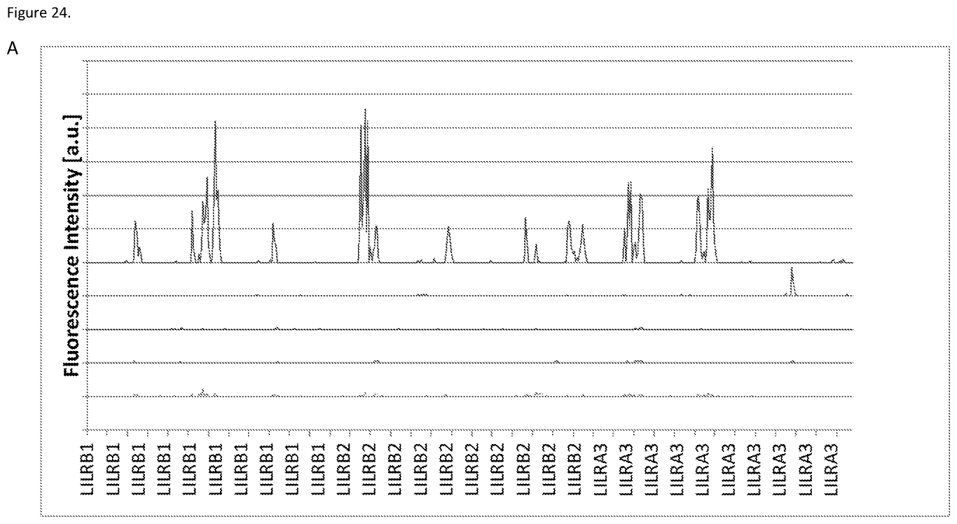

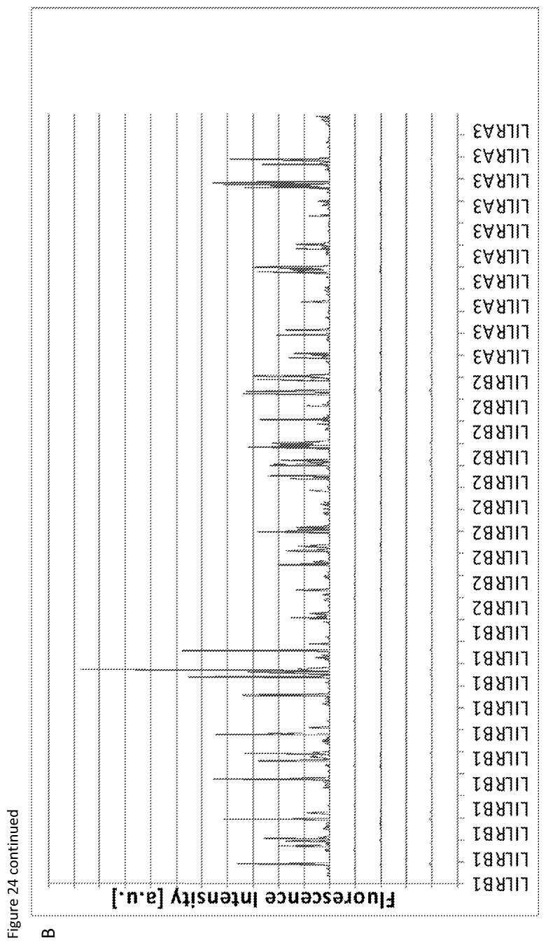

Putative epitopes on D3-D4 regions of human LILRB1, LILRB2, and LILRA3 molecules for Antibodies 1 to 5 were mapped by a CRO; PEPperPRINT; using their proprietary PEPperCHIP® linear and “conformational” peptide microarrays (FIG. 24). Strong binding to peptides microarrays was observed only for Clone 2, suggesting it binds to relatively unstructured epitopes. Two putative epitopes present in all three target proteins corresponded to peptides: Epitope 1 (sequence FVLYKDGERDF (SEQ ID NO: 80) in LILRB1, sequence GYDRFVLYKEGERD (SEQ ID NO: 81) in LILRB2, and sequence YDRFVLYKEWGRD (SEQ ID NO: 82) in LILRA3) and Epitope 2 (sequence SSEWSAPSDPLD (SEQ ID NO: 83) in LILRB1, sequence ECSAPSDPLDI (SEQ ID NO: 84) in LILRB2, and sequence SEWSAPSDPLD (SEQ ID NO: 85) in LILRA3). Binding was also observed to additional putative epitopes: Epitope 3 (sequence LQCVSDVGYD (SEQ ID NO: 86) in LILRB2 and sequence FQCGSDAGYDRF (SEQ ID NO: 87) in LILRA3), and Epitope 4 (sequence FLLTKEGAADDPW (SEQ ID NO: 88) in LILRB1 and sequence AADAPLRLRSIHEY (SEQ ID NO: 89) in LILRB2). Although with a weaker signal, binding to similar peptides was observed for Antibody 1. Antibody 4 was found binding to two putative epitopes; Epitope 5 (sequence RSYGGQYR (SEQ ID NO: 90) in LILRB1 and sequence PVSRSYGGQYRC (SEQ ID NO: 91) in LILRB2).

Weak binding to peptide arrays was observed for Antibody 5 suggesting one putative epitope; Epitope 6 sequence LDILIAGQFYD (SEQ ID NO: 92) in LILRB1, sequence APSDPLDILI (SEQ ID NO: 93) in LILRB2, and sequence PSDPLDILI (SEQ ID NO: 94) in LILRA3). No significant binding to peptide arrays was observed for Antibody 3.

### MDM Reprogramming Using F(Ab′)₂ Dimers and Fab Monomers

F(ab′)2 dimers and Fab monomers were prepared from IgG4P and IgG1 variants of Antibody 2, using FabRICATOR and FabALACTICA kits, respectively, according to the manufacturer's protocols. Purified Ab fragments were used in macrophage re-programming assays as described earlier.

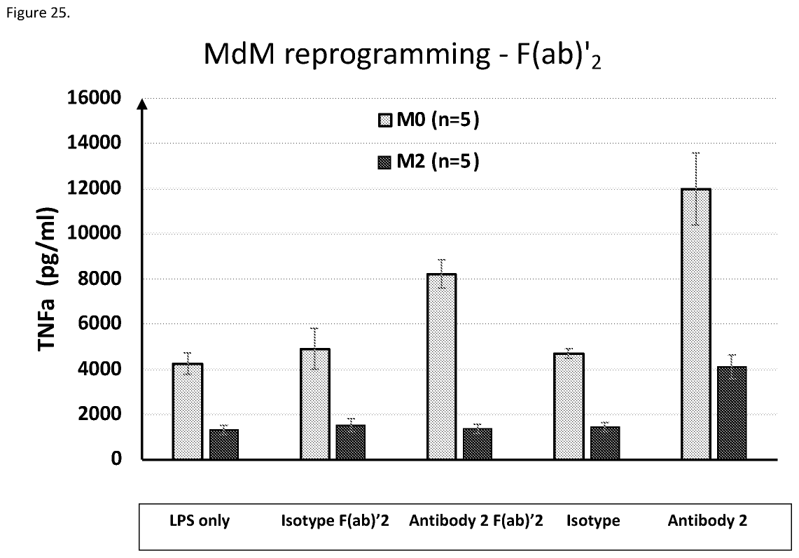

Treatment of M0 macrophages with F(ab′)2 fragment of Antibody 2 for 6h resulted in a partial activation of the cells, as compared to the effects of the intact Antibody 3 Ab and the isotype control, activation of the cells was measured by the release of TNFa. A similar treatment of M2 macrophages did not result in the activation of the cells over that observed with the isotype control. (FIG. 25).

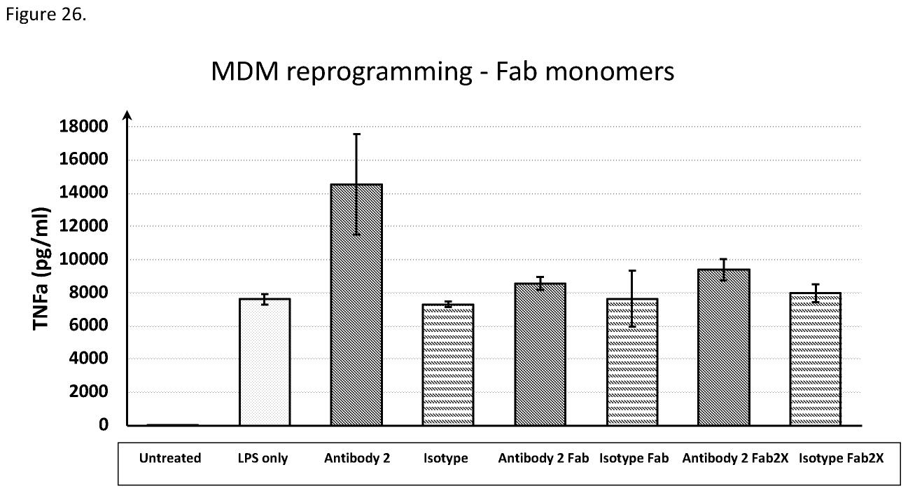

Treatment of M0 macrophages with Fab fragments of Antibody 3 for 6h did not yield enhanced activation (assessed as production of TNFa) when compared to the isotype control production of TNFa. (FIG. 26).

These results indicate that for the optimal re-programming activity, especially under the immunosuppressive M2 conditions, Antibody 2 requires bi-valent binding and the Fc receptor engagement on the macrophage membrane.

In the M2-like Suppression Assay, monocytes were isolated from 3 cryopreserved PBMC donors and cultured with M-CSF and a specific cytokine cocktail (IL-4, IL-10 and TGF-β) to obtain M2-like macrophages. The M2-like macrophages were activated with LPS in the final 4 hours of polarization. At the end of the polarization/activation, the expression of CD163, CD209, CD206, CD86, LILRB1 and LILRB2 was evaluated by flow cytometry. The macrophages were then washed and seeded in 5-plicates in 96-well plates. After overnight resting, CD4+ T cells were added to the plate in a 1:5 macrophage: CD4+ T cell ratio. The T cells in this co-culture were then activated by addition of CD3/CD28 ImmunoCult™ (STEMCELL Technologies) in the presence of the test antibodies at single concentration (10 μg/ml), OPDIVO and a human IgG4 isotype control and reference antibodies and the corresponding isotype control at 1 concentration (10 μg/ml). On day 5 of the co-culture, supernatants were harvested and secreted IFN gamma was evaluated by ELISA. T cell proliferation was assessed by flow cytometry (proliferation dye dilution).

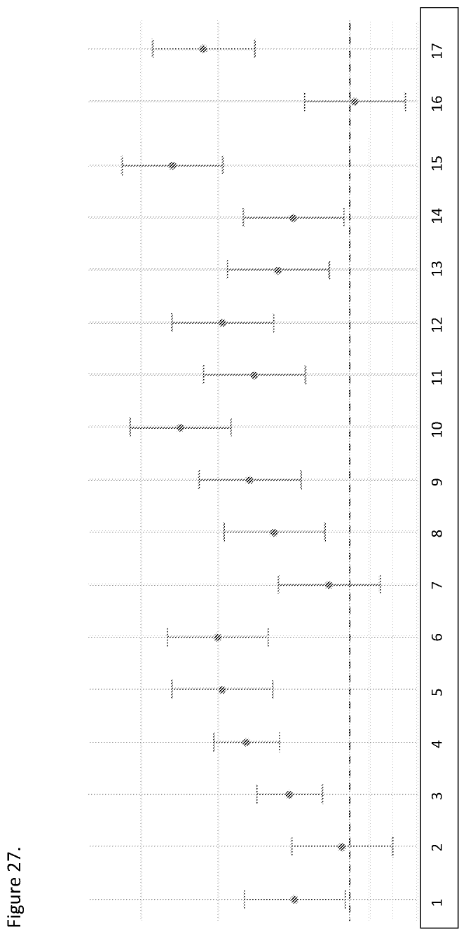

The supernatants were tested by ELISA to measure IFN gamma release. As reported in FIG. 27 the isotype controls show a small significant increase, while we observed significant IFN gamma release using Antibody 2, Antibody 3 and Antibody 4; interestingly enough the IFN gamma production observed with these three antibodies was superior to the reference antibodies tested. Antibody 2 was tested in different IgG versions (IgG1, IgG4, LALA), no significant differences were observed between IgG1 and IgG4, while the LALA format showed a decrease in IFN gamma production.

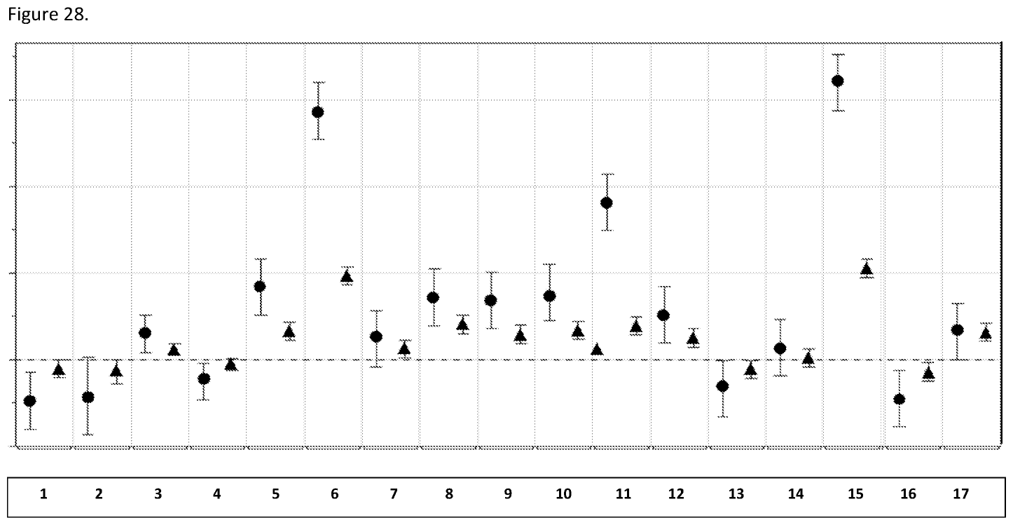

T cell proliferation and expansion was checked by flow cytometry. For T cell proliferation, T cells undergoing proliferation at the start of the assay were assessed. For expansion, a total proliferation index was assessed, this included T cells which were not already proliferating at the start of the assay. The T cell expansion results (FIG. 28) showed that Antibody 2 significantly outperformed the rest of the antibodies tested. Antibody 2 was tested in different IgG versions (IgG1, IgG4, LALA), no significant differences were observed between IgG1 and IgG4 formats, while the LALA format showed a decrease in T cell expansion.

### NKL Killing Assay

NKL is a human natural killer (NK) cell line established from the peripheral blood of a patient with CD3-, CD16+, CD56+, large granular lymphocyte (LGL), kindly provided by Professor Werner Held (University of Lausanne, Switzerland).

Jurkat and Jurkat HLA-G overexpressing cells were used as targets in cytolytic cell assays. The target cells were labeled with CellTracker Deep Red (ThermoFisher) to distinguish them from NKL cells and resuspended at 5×10⁵ cells/ml in assay media

NKL cells were suspended at 1×10⁶ cells/ml in assay media (RPMI 1640 with GlutaMAX, 10% human AB serum, 1% penicillin/streptomycin, 1 mM sodium pyruvate, 1000U/ml of recombinant human IL-2 (rhIL-2) and 100 μl of the NKL cell suspension were added to each well of a 96 well plate. Anti-LILRB1 and/or anti-LILRB2 antibodies and control isotype control were added to the corresponding wells at the same time at a final concentration of 10 μg/ml for a final volume of 200 ul. NKL cells were incubated with the different treatments for 1 hour at 37° C. and subsequently, 100 μl of the target cells were added to the corresponding wells resulting in a NKL to target ratio of 2:1. Plates were cultured for 3 hours at 37° C. followed by centrifugation at 200×g for 3 minutes at room temperature and removal of media.

Each well was resuspended in 100 μl of FACS buffer (2% BSA, 5 mM EDTA-DPBS) and CD56-FITC antibody was added for NKL identification by flow cytometry and incubated at 4° C. for 30 minutes. After antibody incubation, 100 μl FACS buffer were added per well to wash antibody, spin down at 200G for 3 minutes and supernatant was removed. Cells were then resuspended in FACS buffer containing a 1:1,000 dilution of Sytox Blue (ThermoFisher) in order stain cells with compromised cell membranes allowing live cells to be distinguished from dead or damaged cells.

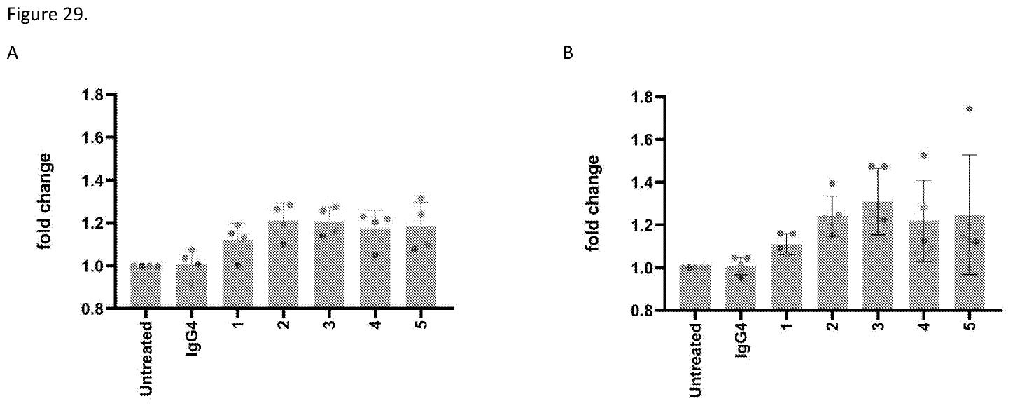

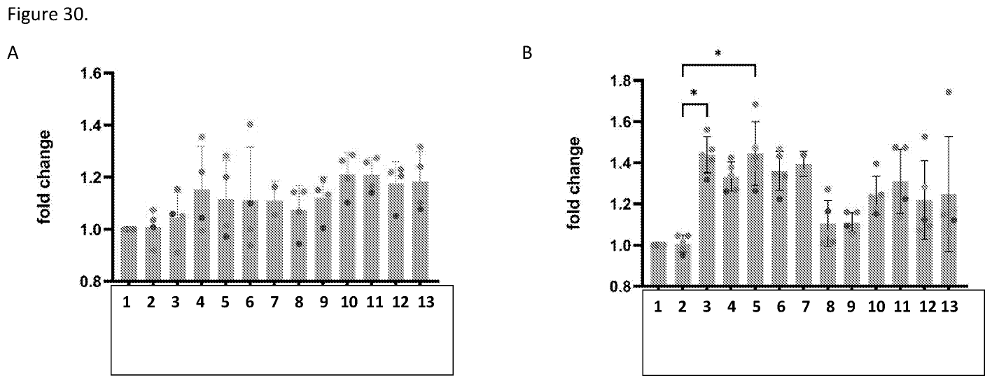

As shown in FIG. 29 and FIG. 30, anti-LILRB1 and/or anti-LILRB2 antibodies of the invention enhanced NKL cytolytic activity compared to the isotype control on wild type Jurkat cells. However, this increase in cytolytic capacity was observed to be higher in the HLA-G over-expressing Jurkat co-cultures.

These results suggest that anti-LILRB1 and anti-LILRB1/LILRB2 antibodies are able to block the interaction between LILRB1 on NKL cells and MHC I molecules on the surface of target cells resulting in the enhancement of NKL killing capacity.

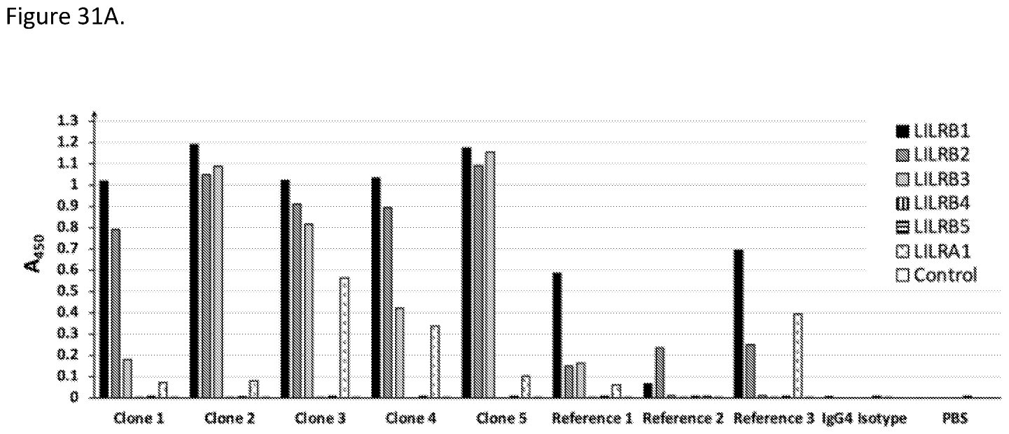

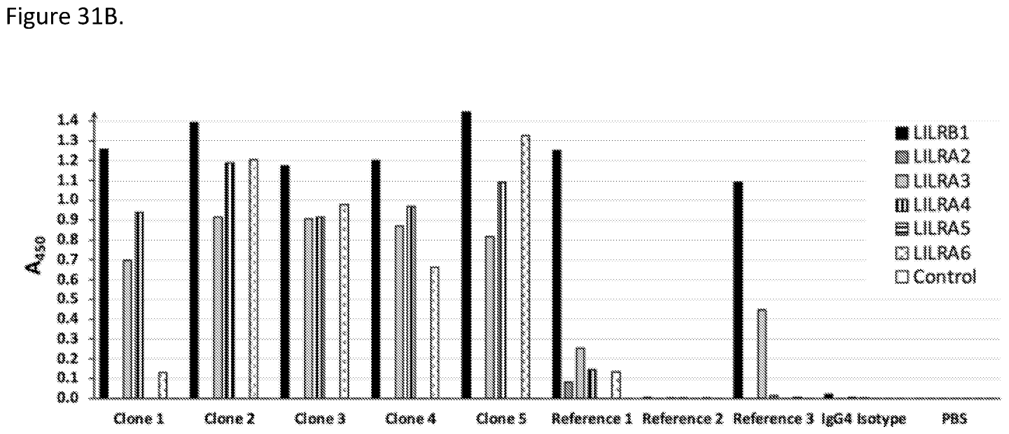

### Binding of Clones 1-5 to all LILR Family Members Tested by Single Point ELISA (FIG. 31).

Binding to the other LILR family members was tested by ELISA using recombinant, full-length ectodomains of LILRA1 (SEQ ID NO: 47), LILRA2 (SEQ ID NO: 48), LILRA3 (SEQ ID NO: 48), LILRA4 (SEQ ID NO: 95), LILRA5 (SEQ ID NO: 96), LILRA6 (SEQ ID NO: 97), LILRB1 (SEQ ID NO: 98), LILRB2 (SEQ ID NO: 99), LILRB3 (SEQ ID NO: 100), LILRB4 (SEQ ID NO: 101), and LILRB5 (SEQ ID NO: 102).

Recombinant LILRB1-5 and LILRA1 (A), or LILRB1 and LILRA2-6 (B) were coated on the ELISA plates. The plates were then blocked with milk in PBST. Next, the plates were incubated with 1 nM of Clone 1-5, Reference 1-3, or IgG4 isotype control. Bound antibodies were detected using HRP-conjugated anti-human Fc antibodies and the chromogenic substrate.

Clones 1-5 bound to LILRB1, LILRB2, LILRB3, LILRA1, LILRA3, LILRA4, and LILRA6.

TABLE 10
Summary of binding characteristics and biological activity reported
for antibodies of the invention (Y = Yes, N = No).

| Antibody | LILRB1 | LILRB2 | LILRB3 | LILRB4 | LILRB5 | LILRA1 | LILRA2 | LILRA3 | LILRA4 | LILRA5 |
|---|---|---|---|---|---|---|---|---|---|---|
| ID | binding | binding | binding | binding | binding | binding | binding | binding | binding | binding |
| 1. | Y | Y | Y | N | N | Y | N | Y | Y | N |
| 2. | Y | Y | Y | N | N | Y | N | Y | Y | N |
| 3. | Y | Y | Y | N | N | Y | N | Y | Y | N |
| 4. | Y | Y | Y | N | N | Y | N | Y | Y | N |
| 5. | Y | Y | Y | N | N | Y | N | Y | Y | N |

| | | | | | | | Binding |
|---|---|---|---|---|---|---|---|
| | | Blocker | | | | LILRB1 | to |
| | | of | | | | D1-D2 | LILRB1 |
| | | HLA--G | Rhesus | | | binding | D1-D4 |
| | | oligomer | & | | | (SEQ | (SEQ |
| | | binding | Cyno | Reprogramming | | ID | ID |
| Antibody | LILRA6 | to | LILRB1/2 | of | | NO: | NO: |
| ID | binding | LILRB1 | binding | macrophage | Phagocytosis | 42) | 41) |
| 1. | Y | N | Y | Y | Y | N | Y |
| 2. | Y | N | Y | Y | N | N | Y |
| 3. | Y | N | Y | Y | Y | N | Y |
| 4. | Y | N | Y | Y | N | N | Y |
| 5. | Y | N | Y | Y | Y | N | Y |

As shown in FIGS. 31 A & B, Antibody Clones 1 to 5, each bound to LILRB1, LILRB2, LILRB3, LILRA1, LILRA3, LILRA4 and LILRA6. At the same time Reference Antibodies 1-3 showed more limited binding specificity. In this context, “binding” denotes that in this assay a signal obtained for LILR protein was at least more than 3 fold higher than that observed for the control protein. To better understand the binding of Antibodies 1-5 to individual family members, Multi-point ELISA was performed and EC₅₀s for the binding were determined.

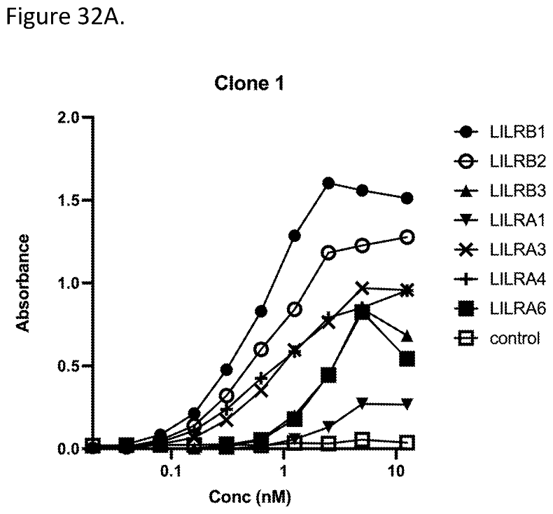

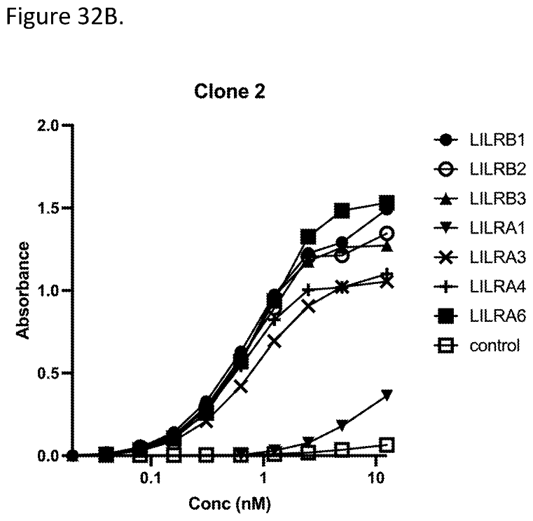

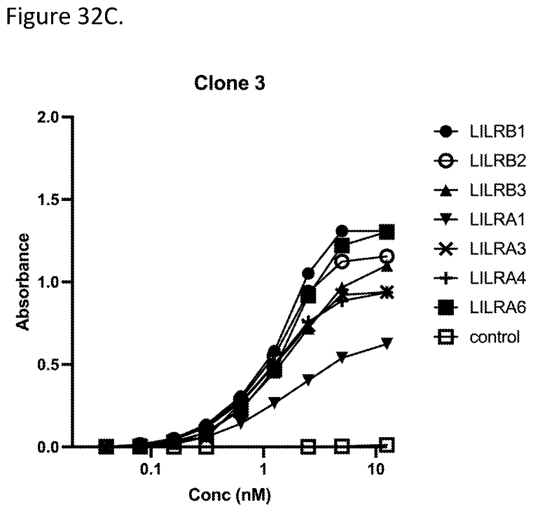

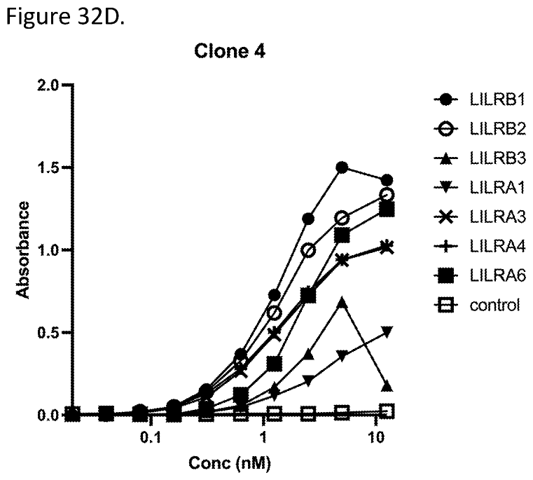

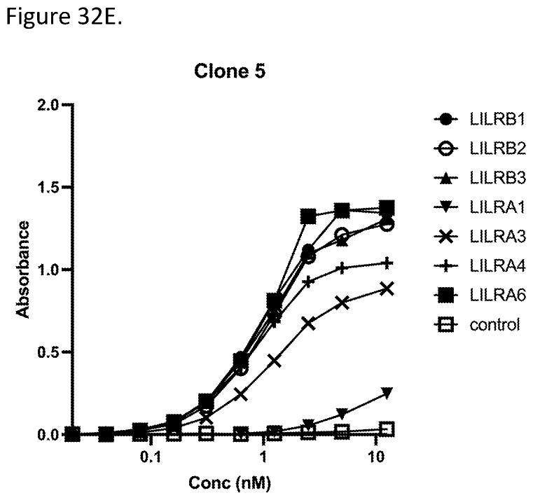

Binding of Clones 1-5 to selected LILR family members tested by multiple point ELISA. (FIG. 32) Recombinant LILRB1, LILRB2, LILRB3, LILRA1, LILRA3, LILRA4, LILRA6, or irrelevant control protein were coated on the ELISA plates. The plates were then blocked with milk in PBST. Next, the plates were incubated with serial dilutions (12.5 nM to 0.02 nM) of Clone 1-5 (A-E). Bound antibodies were detected using HRP-conjugated anti-human Fc antibodies and the chromogenic substrate. The calculated apparent EC₅₀ in nM for each of Antibodies 1 to 5 is shown in Table 11.

TABLE 11
Calculated apparent EC₅₀ in nM for Antibodies 1 to 5.

| Antigen | Antibody 1 | Antibody 2 | Antibody 3 | Antibody 4 | Antibody 5 |
|---|---|---|---|---|---|
| LILRB1 | 0.55 | 0.78 | 1.38 | 1.24 | 0.99 |
| LILRB2 | 0.73 | 0.74 | 1.28 | 1.39 | 1.04 |
| LILRB3 | 2.00 | 0.69 | 1.75 | 1.38 | 0.99 |
| LILRA1 | 2.38 | 8.37 | 1.76 | 4.10 | 9.07 |
| LILRA3 | 0.97 | 0.84 | 1.20 | 1.42 | 1.26 |
| LILRA4 | 0.81 | 0.62 | 1.15 | 1.38 | 0.84 |
| LILRA6 | 1.81 | 0.92 | 1.69 | 2.20 | 0.97 |

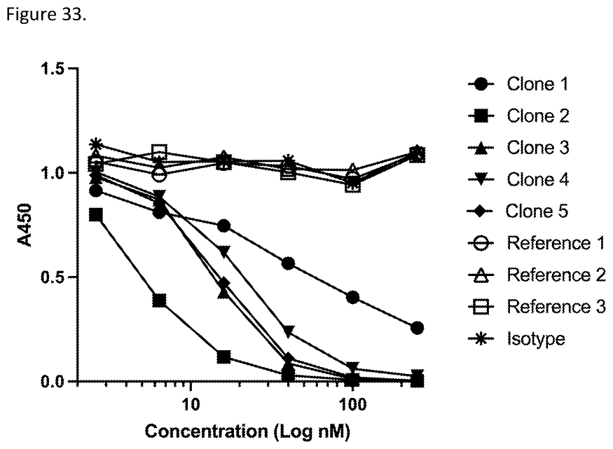

### Competition Between Antibody 2 and Other Antibodies in ELISA. (FIG. 33)

Antibodies 1 to 5 were all found to bind within the D3-D4 fragment of human LILRB1. To investigate if Antibody 2 binds to an epitope similar to or different from the epitope of the other Antibodies (and Reference Antibodies) a competitive ELISA was performed. Plates were coated with human LILRB1 and after blocking incubated with 5 nM of biotinylated Antibody 2 and different concentrations of each unlabelled Antibody 1-5 and the Reference Antibodies 1 to 3. Bound biotinylated Antibody 2 was detected using HRP-conjugated streptavidin and a chromogenic substrate. Unlabelled Antibody 2 competed its biotinylated counterpart with low nM IC₅₀, while Antibodies 1, 3, 4 and 5 yielded IC₅₀ about 10 nM. None of the Reference Abs nor the isotype control could compete with Antibody 2 up to 250 nM concentrations. These data indicate that Antibody 2 binds a unique epitope on LILRB1 that is distal from the epitopes of all of the Reference Abs, but proximal to the epitopes of Antibody 1, 3, 4 and 5.

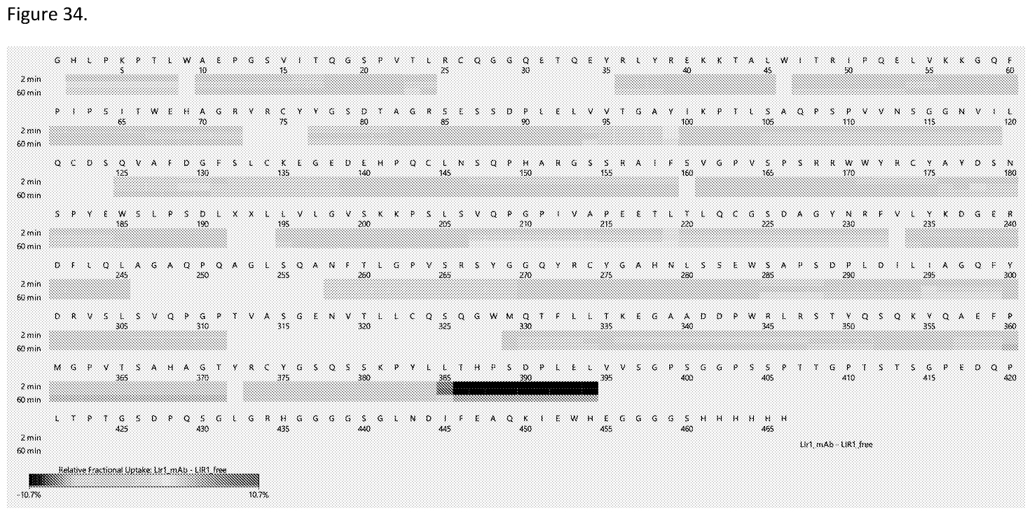

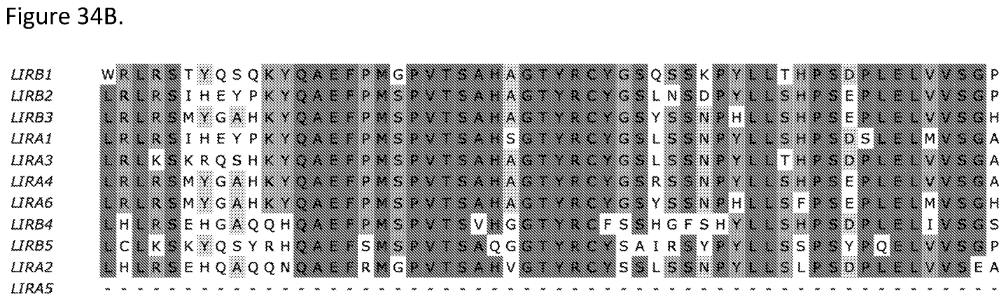

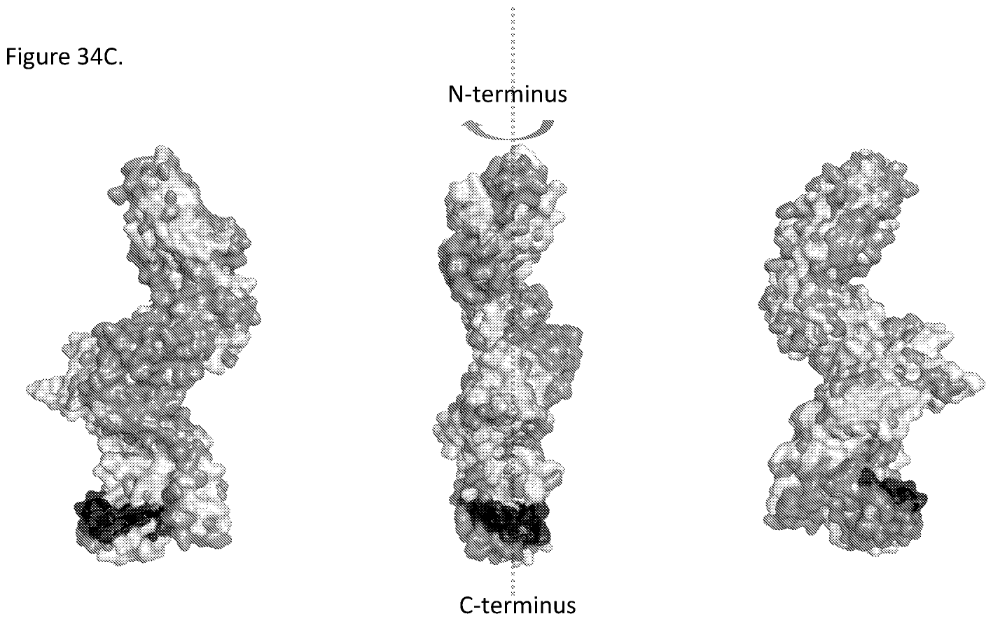

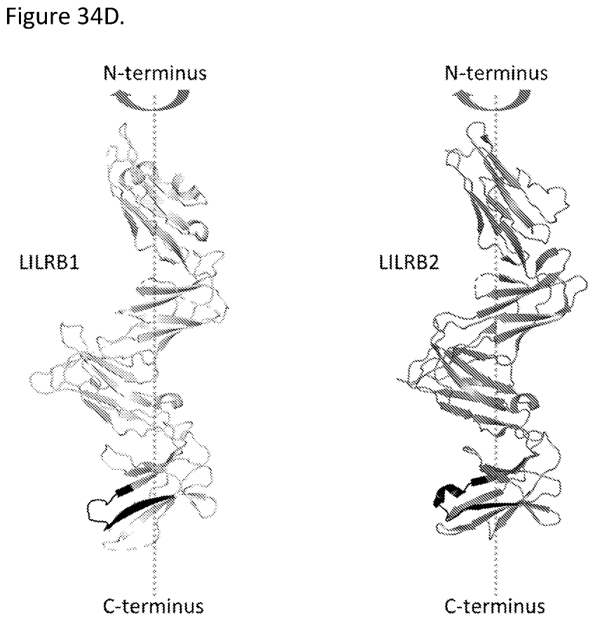

### Epitope Mapping of Antibody 2 on Human LILRB1 by the HDX Method. (FIG. 34)

Experiments were performed using a Trajan automation platform and Waters Cyclic IMS MS. For peptide mapping, recombinant LILRB1-avi-his was diluted to 10 UM in H₂O-based buffer before experiments. For the peptide mapping experiments, 8.5 μL of the protein solution was mixed with 41.5 μL of H₂O based buffer. At the end of the reaction, 45 μl of the sample was mixed with the 45 μL of pre-dispensed quench buffer. The sample was further diluted with 90 μL of quench dilution solution. After dilution, 85 μl of the sample was injected into the sample loop. Samples were run through a Nap2/pepsin or Pep/protease XIII column for 210 seconds at a flow rate of 0.1 mL/min before trapping and desalting, followed by separation through an analytical C18 column at 0.035 mL/min.

For labelling experiments, three different time points were used (2, 10, and 60 minutes) in both the free and bound states. Both the free and the bound state LILRB-avi-his samples were prepared at 10 uM.

PLGS was used to prepare a peptide library, and DynamX 3.0 was used for deuterium uptake analysis.
H₂O Buffer: 20 mM phosphate buffer, 150 mM NaCl, pH 7.4
D₂O/Exchange Buffer: 20 mM phosphate buffer, 150 mM NaCl, pD 7.0
Quench Buffer: 7.6 M Gnd.HCl, 100 mM phosphate buffer, 500 mM TCEP, pH 2.3

The antibody was incubated with the recombinant human LILRB1-avi-his in D₂O 20 mM Sodium Phosphate, 150 mM NaCl, pH 7.4 for different periods of time. The protein mixture was then digested with proteases and analysed on the Leap HDX auto sampler and Waters Cyclic IMS MS machine. (A) The relative changes in deuterium uptake (lighter indicating less uptake, darker indicating more uptake) after for 2, 10, and 60 minutes (top to bottom bars under the sequence) in proteolytic peptides is indicated by the heatmap below the sequence of the LILRB1 (FIG. 34). The putative epitope for Antibody 2 (sequence AEFPMGPVTSAHAGT (SEQ ID NO: 78)) is underlined with a solid line and the other region possibly involved in the Antibody 2 binding (sequence LTHPSDPLEL (SEQ ID NO: 79)) is underlined with a dashed line. (B) Alignment of all 11 human LILR family members with the putative epitope identified by HDX outlined in solid line and the other region possibly involved in the Antibody 2 binding is outlined with a dotted line. The LILR family members bound by Antibodies 1-5 are in dashed line box. (C) Aligned 3D structures (surface view) of human LILRB1 (light grey) and LILRB2 (dark grey) with the putative epitope in domain 4 highlighted black. (D) A ribbon view of LILRB1 (left) and LILRB2 (right) 3D structures with the putative Antibody 2 epitope in domain 4 highlighted black.

### MDM Methods

Monocytes for monocyte-derived macrophage (MDM) differentiation were either isolated from fresh PBMCs by CD14 positive selection using the MACS isolation system (Miltenyi Biotec 130-050-201) or sourced commercially as cryopreserved peripheral blood monocytes (StemCell Technologies 70034/200-0166).

MDMs were differentiated by plating at 1-2*10⁶ monocytes per well in 6 well UpCell plates (Thermo Scientific 174901) in MDM media (RPMI 1640 with Glutamax (Gibco 61870010), supplemented with 10% heat-inactivated fetal bovine serum (Life Technologies A3840402), 100 U/ml Penicillin/Streptomycin (Gibco 15140-122) and 100 ng/ml recombinant human M-CSF (Biolegend 574806)) for 7-10 days. To polarize MDMs to an immunosuppressive phenotype, 50 ng/ml each of recombinant human IL-10 (Peprotech 200-10) and TGF-β1 (Peprotech 100-21) were added at the point of seeding and for the whole duration of the differentiation and subsequent assays.

For MDM reprogramming assays, MDMs were re-plated in MDM media after 6-12 days of differentiation at a density of 31,000-94,000 cells/cm². Immunosuppressed MDMs were supplemented with 50 ng/ml IL-10 and TGF-β1 as before. Before adding activating stimuli, MDMs were pre-treated with 10 μg/ml of the monoclonal antibodies for 1 hour. Antibodies tested were Antibody 2, Reference Antibody 1, Reference Antibody 2, Reference Antibody 3 and Reference Antibody 7. Antibody 7 is an LILRB2-specific antibody. Human anti-HEL (hen egg lysozyme) IgG4P was used as isotype control. LPS was then added at 1 ng/ml (resting MDMs, M0) or 10 ng/ml (immunosuppressed MDMs, M2), recombinant human IL-1ß (Peprotech 200-01B) was added at 10 ng/ml, HMGB1 peptide (FKDPNAPKRLPSAFFLFCSE (SEQ ID NO: 105), produced by GenScript) was added at 30 μg/ml, c-di-AMP (Invivogen tlrl-nacda2r) was added at 10 μg/ml, poly(I:C) (Invivogen, LMW-tlrl-picw, HMW-tlrl-pic) was added at 10 μg/ml and R848/resiquimod (Invivogen tlrl-r848) was added at 0.5 μg/ml. Media was collected 5 hours after LPS addition or 24 hours after addition of all other stimuli. TNFα and GM-CSF release was measured by Duoset ELISA (R&D Systems).

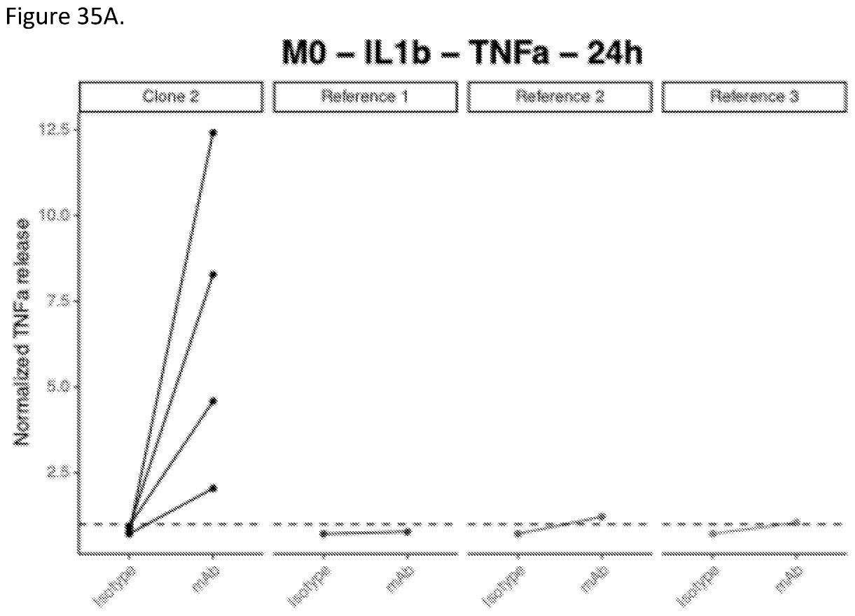

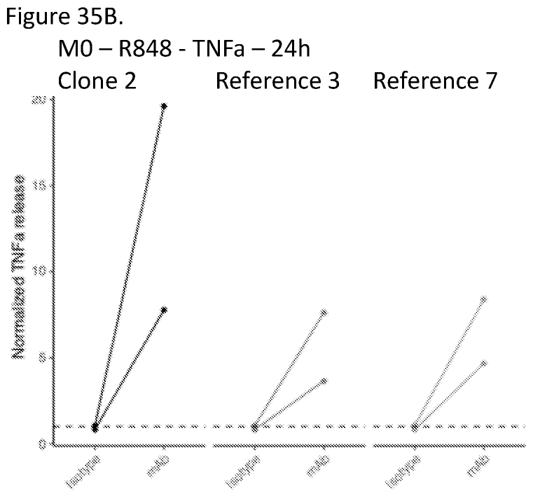

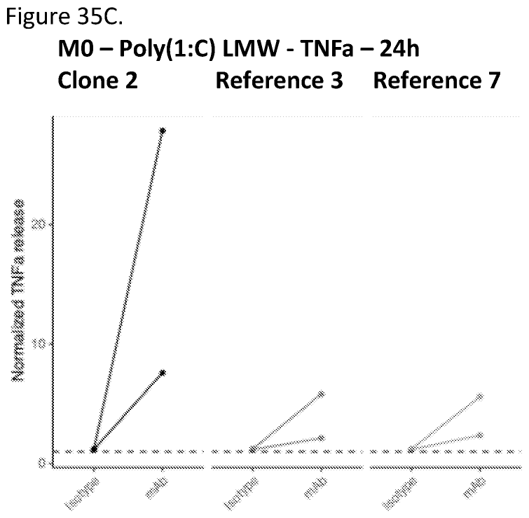

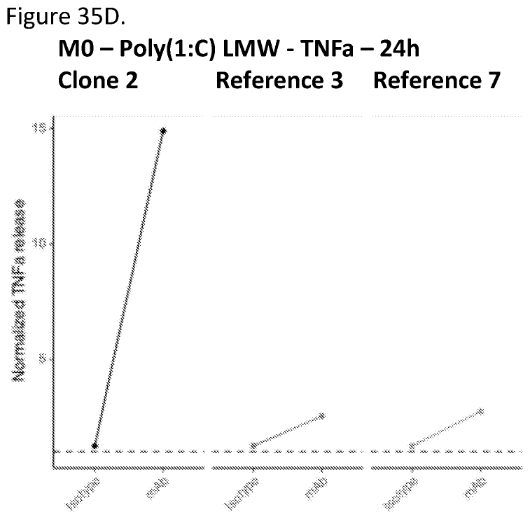

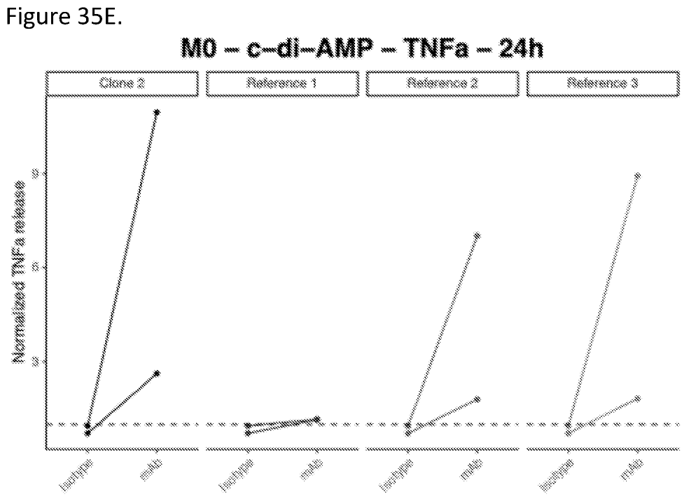

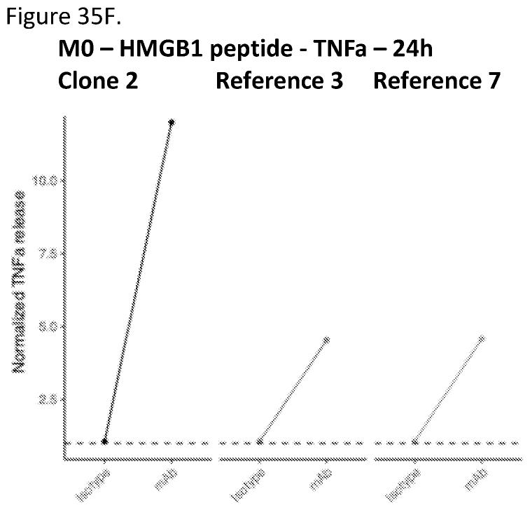

Antibody 2 Enhances Secretion of TNFα by Human Blood Monocyte Derived Macrophages (MDMs) Upon Induction with Various Stimuli. (FIG. 35)

M0 polarized MDMs were incubated for 1 h with 10 μg/ml of Antibody 2 and then stimulated with various compounds: (A) IL-1β at 10 ng/ml, (B) TLR7/TLR8 agonist R848 at 0.5 g/ml, (C) TLR3 ligand LMW Poly(I:C) at 10 μg/ml, (D) TLR3 ligand HMW Poly(I:C) at 10 g/ml, (E) STING agonist c-di-AMP at, and (F) TRL4 agonistic HMGB1-derived peptide at 30 μg/ml. Data presented are background normalized.

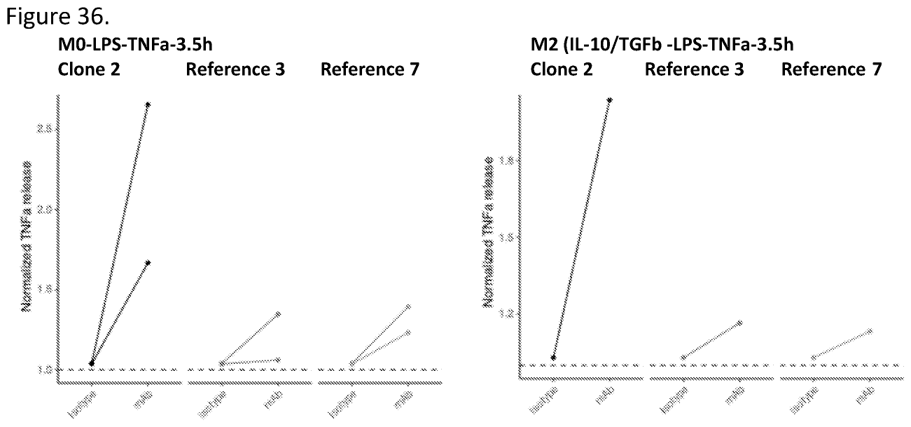

Antibody 2 Enhances Secretion of TNFα by MDMs Polarized to Either M0 or M2 Phenotype (with IL-10 and TGFβ) Co-Cultured with A375 Melanoma Cells Upon Induction with LPS. (FIG. 36)

MDMs were co-cultured with A375 cells for 4.5 hours were incubated with 10 μg/ml of Antibody 2 for 1 hour and then stimulated with LPS (1 ng/ml and 10 ng/ml, for M0 and M2 MDMs, respectively) for 3.5 hours. Data presented are background normalized.

### REFERENCES

Barkal et al., “Engagement of MHC class I by the inhibitory receptor LILRB1 suppresses macrophages and is a target of cancer immunotherapy”, Nat. Immunol. (2018) January; 19 (I): 76-84.
Chen et al JCI (2018) J Clin Invest. 2018; 128 (12): 5647-5662. https://doi.org/10.1172/JCI97570.
Chen et al, JITC, (2020) Chen H, Chen Y, Deng M, et al. Antagonistic anti-LILRB1 monoclonal antibody regulates antitumor functions of natural killer cells. Journal for Immuno Therapy of Cancer 2020; 8: e000515. doi: 10.1136/jitc-2019-000515.
Coxon et al. Blood (2017) 129 (26): 3407-3418.
Kontermann (2012) Mabs 4 (2): 182-97.
Kuroki et al. Human Molecular Genetics, 2005, Vol. 14, 2469-2480.
Lopez-Yrigoyen, M., May, A., Ventura, T., Taylor, H., Fidanza, A., Cassetta, L., Pollard, J. W., Forrester, L. M. Production and Characterization of Human Macrophages from Pluripotent Stem Cells. J. Vis. Exp. (158), e61038, doi: 10.3791/61038 (2020).
Lopez-Yrigoyen, M., et al. A human iPSC line capable of differentiating into functional macrophages expressing ZsGreen: A tool for the study and in vivo tracking of therapeutic cells. Philosophical Transactions of the Royal Society B: Biological Sciences. 373, (1750), (2018).
Lopez-Yrigoyen, M., et al. Genetic programming of macrophages generates an in vitro model for the human erythroid island niche. Nature Communications. 10, (1), 881 (2019).
Mulder et al. Immunity. 2021 Aug. 10; 54 (8): 1883-1900.e5. doi: 10.1016/j.immuni.2021.07.007. Epub 2021 Jul. 30.
Spiess et al. (2015) Mol Immunol 67:95-106.
van Wilgenburg, B., Browne, C., Vowles, J., Cowley, S. A. Efficient, long term production of monocyte-derived macrophages from human pluripotent stem cells under partly-defined and fully-defined conditions. PLOS One. 8, (8), 71098 (2013).
Wang et al. (2020) Cell Mol Immunol. 2020 September; 17 (9): 966-975. doi: 10.1038/s41423-019-0258-5. Epub 2019 Jul. 4.
Yeboah et al. JCI 2020 JCI Insight. 2020 Sep. 1; 5 (18): e141593. doi: 10.1172/jci.insight.141593.

Sequence Listing Information

SEQ ID NO: 1 Antibody 1 HCDR1 DYYMS
SEQ ID NO: 2 Antibody 1 HCDR2 YISSSGSIKKYADSVKG
SEQ ID NO: 3 Antibody 1 HCDR3 TNWHFDY
SEQ ID NO: 4 Antibody 1 LCDR1 RASQSVSSSYLA
SEQ ID NO: 5 Antibody 1 LCDR2 GASTRAT
SEQ ID NO: 6 Antibody 1 LCDR3 QQYYSTPFT
SEQ ID NO: 7 Antibody 1 VH
QVQLVESGGGLVKPGGSLRLSCAASGFTFSDYYMSWIRQAPGKGLEWVSYISSSGSIKKYADSVKGRFTI
SRDNAKNSLYLQMNSLRGEDTAVYYCARTNWHFDYWGQGTLVTVSS
SEQ ID NO: 8 Antibody 1 VL
EIVLTQSPGTLSLSPGERATLSCRASQSVSSSYLAWYQQKPGQAPRLLIFGASTRATGIPDRFSGSGSGT
DFTLTISRLEPEDVAVYYCQQYYSTPFTFGPGTKVDIK
SEQ ID NO: 9 Antibody 2 HCDR1 DYYMS
SEQ ID NO: 10 Antibody 2 HCDR2 YISPSGSTIFYADSVKG
SEQ ID NO: 11 Antibody 2 HCDR3 DRVRLFDY
SEQ ID NO: 12 Antibody 2 LCDR1 RASQSVSSSYLA
SEQ ID NO: 13 Antibody 2 LCDR2 GASTRAT
SEQ ID NO: 14 Antibody 2 LCDR3 QQRSNWPIT
SEQ ID NO: 15 Antibody 2 VH
QVQLVESGGDLVKPGGSLRLSCAASGFTFRDYYMSWIRQAPGKGLEWVSYISPSGSTIFYADSVKGRFTI
SRDNAKNSLYLQMNSLRAEDTAVYYCAKDRVRLFDYWGQGTLVTVSS
SEQ ID NO: 16 Antibody 2 VL
EIVLTQSPATLSLSPGERATLSCRASQSVSSSYLAWYQQKPGQAPRLLIYGASTRATGIPARFSGSGSGT
DFTLTISRLEPEDFAVYYCQQRSNWPITFGQGTRLEIK
SEQ ID NO: 17 Antibody 3 HCDR1 SYGIS
SEQ ID NO: 18 Antibody 3 HCDR2 WISAYNGNTNYAQKLQG
SEQ ID NO: 19 Antibody 3 HCDR3 SYSGSHWWFDP
SEQ ID NO: 20 Antibody 3 LCDR1 KSSQSVLYSSNNNNYLA
SEQ ID NO: 21 Antibody 3 LCDR2 WAFSRES
SEQ ID NO: 22 Antibody 3 LCDR3 QQYYSTPPT
SEQ ID NO: 23 Antibody 3 VH
QVQLVQSGAEVKKPGASVKVSCKASGYTFTSYGISWVRQAPGQGLEWMGWISAYNGNTNYAQKLQGR
VTMTTDTSTSTAYMELRSLRSDDTAVYYCARSYSGSHWWFDPWGQGTLVTVSS
SEQ ID NO: 24 Antibody 3 VL
DIVMTQSPDSLAVSLGERATINCKSSQSVLYSSNNNNYLAWYQRKPGQPPKLLINWAFSRESGVPDRFS
GSGSGTDFTLTISSLQAEDVAVYYCQQYYSTPPTFGQGTKVEIK
SEQ ID NO: 25 Antibody 4 HCDR1 SYGIS
SEQ ID NO: 26 Antibody 4 HCDR2 WISAYNGNTNYAQKLQG
SEQ ID NO: 27 Antibody 4 HCDR3 SYSGSYWWFDP
SEQ ID NO: 28 Antibody 4 LCDR1
KSSQSVLYSSNNNNYLA
SEQ ID NO: 29 Antibody 4 LCDR2
WASTRES
SEQ ID NO: 30 Antibody 4 LCDR3
QQYYSTPLT
SEQ ID NO: 31 Antibody 4 VH
QVQLVQSGAEVKKPGASVKVSCKASGYTFTSYGISWVRQAPGQGLEWMGWISAYNGNTNYAQKLQGR
VTMTTDTSTSTAYMELRSLRSDDTAVYYCARSYSGSYWWFDPWGQGTLVTVSS
SEQ ID NO: 32 Antibody 4 VL
DIVMTQSPDSLAVSLGERATINCKSSQSVLYSSNNNNYLAWYQQKPGQPPKLLINWASTRESGVPDRFS
GSGSGTDFTLTISSLQAEDVAVYYCQQYYSTPLTFGGGTKVEIK
SEQ ID NO: 33 Antibody 5 HCDR1 DYYMT
SEQ ID NO: 34 Antibody 5 HCDR2 YISISGITRYYADSVKG
SEQ ID NO: 35 Antibody 5 HCDR3 DQTGYFDY
SEQ ID NO: 36 Antibody 5 LCDR1 RASQSFSSNLA
SEQ ID NO: 37 Antibody 5 LCDR2 GASTRAT
SEQ ID NO: 38 Antibody 5 LCDR3 QQYINWPHT
SEQ ID NO: 39 Antibody 5 VH
QVQLVESGGGLVKPGGSLRLSCAASGFTFSDYYMTWIRQAPGKGLEWVSYISISGITRYYADSVKGRFTI
SRDNAKNSLYLQMNSLRAEDTAVYYCARDQTGYFDYWGQGTLVTVSS
SEQ ID NO: 40 Antibody 5 VL
DIVMTQSPATLSVSPGEKTTLSCRASQSFSSNLAWYQQKPGQAPRLLIYGASTRATGIPARFSGSGSGTE
FTLTISSLQSEDFAVYYCQQYINWPHTFGQGTKVEIK
SEQ ID NO: 41 (human LILRB1 ectodomain fused to mIgG2a Fc)
GHLPKPTLWAEPGSVITQGSPVTLRCQGGQETQEYRLYREKKTALWITRIPQELVKKGQFPIPSITWEHA
GRYRCYYGSDTAGRSESSDPLELVVTGAYIKPTLSAQPSPVVNSGGNVILQCDSQVAFDGFSLCKEGED
EHPQCLNSQPHARGSSRAIFSVGPVSPSRRWWYRCYAYDSNSPYEWSLPSDLLELLVLGVSKKPSLSV
QPGPIVAPEETLTLQCGSDAGYNRFVLYKDGERDFLQLAGAQPQAGLSQANFTLGPVSRSYGGQYRCY
GAHNLSSEWSAPSDPLDILIAGQFYDRVSLSVQPGPTVASGENVTLLCQSQGWMQTFLLTKEGAADDPW
RLRSTYQSQKYQAEFPMGPVTSAHAGTYRCYGSQSSKPYLLTHPSDPLELVVSGPSGGPSSPTTGPTST
SGPEDQPLTPTGSDPQSGLGRHGGGGSEPRGPTIKPCPPCKCPAPNLLGGPSVFIFPPKIKDVLMISLSPI
VTCVVVDVSEDDPDVQISWFVNNVEVHTAQTQTHREDYNSTLRVVSALPIQHQDWMSGKEFKCKVNNK
DLPAPIERTISKPKGSVRAPQVYVLPPPEEEMTKKQVTLTCMVTDFMPEDIYVEWTNNGKTELNYKNTEP
VLDSDGSYFMYSKLRVEKKNWVERNSYSCSVVHEGLHNHHTTKSFSRTPGK
SEQ ID NO: 42 Human LILRB1 (D1-D2) fused to mouse IgG2a Fc
PKPTLWAEPGS VITQGSPVTL RCQGGQETQE YRLYREKKTA LWITRIPQEL
VKKGQFPIPS ITWEHAGRYR CYYGSDTAGR SESSDPLELV VTGAYIKPTL
SAQPSPVVNS GGNVILQCDS QVAFDGFSLC KEGEDEHPQC LNSQPHARGS
SRAIFSVGPV SPSRRWWYRC YAYDSNSPYE WSLPSDLLEL LVLGVSGGGG
SEPRGPTIKP CPPCKCPAPN LLGGPSVFIF PPKIKDVLMI SLSPIVTCVV
VDVSEDDPDV QISWFVNNVE VHTAQTQTHR EDYNSTLRVV SALPIQHQDW
MSGKEFKCKV NNKDLPAPIE RTISKPKGSV RAPQVYVLPP PEEEMTKKQV
TLTCMVTDFM PEDIYVEWTN NGKTELNYKN TEPVLDSDGS YFMYSKLRVE
KKNWVERNSY SCSVVHEGLH NHHTTKSFSR TPGK
SEQ ID NO: 43 Rhesus monkey (Macaca mulatta) LILRB ectodomain fused to mIgG2a Fc
SRTRVQAGTFPKPTLWAEPGSMISKGSPVTLRCQGSLPVQDYRLQREKKTASWVRRIQQELVKKGYFPI
ASITSEHAGQYRCQYYSHSWWSEPSDPLELVVTGAYSKPTLSALPSPVVASGGNVTLQCDSQVAGGFVL
CKEGEDEHPQCLNSQPHTRGSSRAVFSVGPVSPSRRWSYRCYGYDSRSPYVWSLPSDLLELLVPGVSK
KPSLSVQPGPVVAPGDKLTLQCGSDAGYNRFALYKEGERDFLQRPGRQPQAGLSQANFLLDPVRRSHG
GQYRCSGAHNLSSEWSAPSDPLDILIAGQIRGRPSLLVQPGPTVVSGENVTLLCQSSWQFHVFLLTQAG
AADAHLHLRSMYKYPKYQAEFPMSPVTSAHAGTYRCYGSHSSDSYLLSIPSDPLELVVSGPGGGGGSEP
RGPTIKPCPPCKCPAPNLLGGPSVFIFPPKIKDVLMISLSPIVTCVVVDVSEDDPDVQISWFVNNVEVHTAQ
TQTHREDYNSTLRVVSALPIQHQDWMSGKEFKCKVNNKDLPAPIERTISKPKGSVRAPQVYVLPPPEEEM
TKKQVTLTCMVTDFMPEDIYVEWTNNGKTELNYKNTEPVLDSDGSYFMYSKLRVEKKNWVERNSYSCS
VVHEGLHNHHTTKSFSRTPGK
SEQ ID NO: 44 Cynomolgus monkey (Macaca fascicularis) LILRB ectodomain fused to mIgG2a
MGWSCIILFLVATATGVHSGILPKPMLWAEPDRVITQGSPVTLRCQGNLEALGYHLYRERKSASWITLIRP
ELVKKGQFPIPSITWEDAGRYRCQYYSHSWWSEPSDPLELVVTGAYRKPTLSALPSPVVASGGNVTLQC
DSQVPFDGFILCKEGEDEHPQRLNCQSHARGWSRAVFSVGPVSPSRRWSYRCYGYDSRSPYVWSLPS
DLLELLVPGVSKKPSLSVQPGPVVAPGEKLTLQCGSDAGYDRFALYKEGEGEFLQRPGRQPQAGLSQA
NFPLGPVSRSHGGQYRCSGAHNLSSEWSAPSDPLDILISGQFHDVVSLSVQPGPTVASGENVTLLCQSR
AQFHTFLLTREGAAHPPLHLRSEHQAQQYQAEFPMSPVTSAHAGTYRCYGSLSSNPYLLTHPSEPLELV
VSGPSRSPSPPLTGPTPTPAGPEDQSLNPTGSDPQSGLGRHLGVGGGGSEPRGPTIKPCPPCKCPAPN
LLGGPSVFIFPPKIKDVLMISLSPIVTCVVVDVSEDDPDVQISWFVNNVEVHTAQTQTHREDYNSTLRVVS
ALPIQHQDWMSGKEFKCKVNNKDLPAPIERTISKPKGSVRAPQVYVLPPPEEEMTKKQVTLTCMVTDFM
PEDIYVEWTNNGKTELNYKNTEPVLDSDGSYFMYSKLRVEKKNWVERNSYSCSVVHEGLHNHHTTKSFS
RTPGK
SEQ ID NO: 45 Human full length LILRB1
MTPILTVLICLGLSLGPRTHVQAGHLPKPTLWAEPGSVITQGSPVTLRCQGGQETQEYRLYREKKTALWIT
RIPQELVKKGQFPIPSITWEHAGRYRCYYGSDTAGRSESSDPLELVVTGAYIKPTLSAQPSPVVNSGGNVI
LQCDSQVAFDGFSLCKEGEDEHPQCLNSQPHARGSSRAIFSVGPVSPSRRWWYRCYAYDSNSPYEWS
LPSDLLELLVLGVSKKPSLSVQPGPIVAPEETLTLQCGSDAGYNRFVLYKDGERDFLQLAGAQPQAGLSQ
ANFTLGPVSRSYGGQYRCYGAHNLSSEWSAPSDPLDILIAGQFYDRVSLSVQPGPTVASGENVTLLCQS
QGWMQTFLLTKEGAADDPWRLRSTYQSQKYQAEFPMGPVTSAHAGTYRCYGSQSSKPYLLTHPSDPL
ELVVSGPSGGPSSPTTGPTSTSGPEDQPLTPTGSDPQSGLGRHLGVVIGILVAVILLLLLLLLLFLILRHRR
QGKHWTSTQRKADFQHPAGAVGPEPTDRGLQWRSSPAADAQEENLYAAVKHTQPEDGVEMDTRSPH
DEDPQAVTYAEVKHSRPRREMASPPSPLSGEFLDTKDRQAEEDRQMDTEAAASEAPQDVTYAQLHSLT
LRREATEPPPSQEGPSPAVPSIYATLAIH
SEQ ID NO: 46 Human full length LILRB2
MTPIVTVLICLGLSLGPRTHVQTGTIPKPTLWAEPDSVITQGSPVTLSCQGSLEAQEYRLYREKKSASWIT
RIRPELVKNGQFHIPSITWEHTGRYGCQYYSRARWSELSDPLVLVMTGAYPKPTLSAQPSPVVTSGGRV
TLQCESQVAFGGFILCKEGEEEHPQCLNSQPHARGSSRAIFSVGPVSPNRRWSHRCYGYDLNSPYVWS
SPSDLLELLVPGVSKKPSLSVQPGPVVAPGESLTLQCVSDVGYDRFVLYKEGERDLRQLPGRQPQAGLS
QANFTLGPVSRSYGGQYRCYGAHNLSSECSAPSDPLDILITGQIRGTPFISVQPGPTVASGENVTLLCQS
WRQFHTFLLTKAGAADAPLRLRSIHEYPKYQAEFPMSPVTSAHAGTYRCYGSLNSDPYLLSHPSEPLELV
VSGPSMGSSPPPTGPISTPAGPEDQPLTPTGSDPQSGLGRHLGVVIGILVAVVLLLLLLLLLFLILRHRRQG
KHWTSTQRKADFQHPAGAVGPEPTDRGLQWRSSPAADAQEENLYAAVKDTQPEDGVEMDTRAAASEA
PQDVTYAQLHSLTLRRKATEPPPSQEREPPAEPSIYATLAIH
SEQ ID NO: 47 Human LILRA1 ectodomain
PRTHVQAGTLPKPTLWAEPGSVITQGSPVTLWCQGILETQEYRLYREKKTAPWITRIPQEIVKKGQFPIPSI
TWEHTGRYRCFYGSHTAGWSEPSDPLELVVTGAYIKPTLSALPSPVVTSGGNVTLHCVSQVAFGSFILCK
EGEDEHPQCLNSQPRTHGWSRAIFSVGPVSPSRRWSYRCYAYDSNSPHVWSLPSDLLELLVLGVSKKP
SLSVQPGPIVAPGESLTLQCVSDVSYDRFVLYKEGERDFLQLPGPQPQAGLSQANFTLGPVSRSYGGQY
RCSGAYNLSSEWSAPSDPLDILIAGQFRGRPFISVHPGPTVASGENVTLLCQSWGPFHTFLLTKAGAADA
PLRLRSIHEYPKYQAEFPMSPVTSAHSGTYRCYGSLSSNPYLLSHPSDSLELMVSGAAETLSPPQNKSDS
KAGAANTLSPSQNKTASHPQDYTVEN
SEQ ID NO: 48 Human LILRA2 ectodomain
GHLPKPTLWAEPGSVIIQGSPVTLRCQGSLQAEEYHLYRENKSASWVRRIQEPGKNGQFPIPSITWEHAG
RYHCQYYSHNHSSEYSDPLELVVTGAYSKPTLSALPSPVVTLGGNVTLQCVSQVAFDGFILCKEGEDEHP
QRLNSHSHARGWSWAIFSVGPVSPSRRWSYRCYAYDSNSPYVWSLPSDLLELLVPGVSKKPSLSVQPG
PMVAPGESLTLQCVSDVGYDRFVLYKEGERDFLQRPGWQPQAGLSQANFTLGPVSPSHGGQYRCYSA
HNLSSEWSAPSDPLDILITGQFYDRPSLSVQPVPTVAPGKNVTLLCQSRGQFHTFLLTKEGAGHPPLHLR
SEHQAQQNQAEFRMGPVTSAHVGTYRCYSSLSSNPYLLSLPSDPLELVVSEAAETLSPSQNKTDSTTTS
LGQHPQDYTVEN
SEQ ID NO: 49 Human full length LILRA3
GPLPKPTLWAEPGSVITQGSPVTLRCQGSLETQEYHLYREKKTALWITRIPQELVKKGQFPILSITWEHAG
RYCCIYGSHTVGLSESSDPLELVVTGAYSKPTLSALPSPVVTSGGNVTIQCDSQVAFDGFILCKEGEDEHP
QCLNSHSHARGSSRAIFSVGPVSPSRRWSYRCYGYDSRAPYVWSLPSDLLGLLVPGVSKKPSLSVQPG
PVVAPGEKLTFQCGSDAGYDRFVLYKEWGRDFLQRPGRQPQAGLSQANFTLGPVSRSYGGQYTCSGA
YNLSSEWSAPSDPLDILITGQIRARPFLSVRPGPTVASGENVTLLCQSQGGMHTFLLTKEGAADSPLRLKS
KRQSHKYQAEFPMSPVTSAHAGTYRCYGSLSSNPYLLTHPSDPLELVVSGAAETLSPPQNKSDSKAGE
SEQ ID NO: 50 Human LILRB1(L68P/A93T) ectodomain fused to mIgG2aFc
GHLPKPTLWAEPGSVITQGSPVTLRCQGGQETQEYRLYREKKTAPWITRIPQELVKKGQFPIPSITWEHT
GRYRCYYGSDTAGRSESSDPLELVVTGAYIKPTLSAQPSPVVNSGGNVILQCDSQVAFDGFSLCKEGED
EHPQCLNSQPHARGSSRAIFSVGPVSPSRRWWYRCYAYDSNSPYEWSLPSDLLELLVLGVSKKPSLSV
QPGPIVAPEETLTLQCGSDAGYNRFVLYKDGERDFLQLAGAQPQAGLSQANFTLGPVSRSYGGQYRCY
GAHNLSSEWSAPSDPLDILIAGQFYDRVSLSVQPGPTVASGENVTLLCQSQGWMQTFLLTKEGAADDPW
RLRSTYQSQKYQAEFPMGPVTSAHAGTYRCYGSQSSKPYLLTHPSDPLELVVSGPSGGPSSPTTGPTST
SGPEDQPLTPTGSDPQSGLGRHGGGGSEPRGPTIKPCPPCKCPAPNLLGGPSVFIFPPKIKDVLMISLSPI
VTCVVVDVSEDDPDVQISWFVNNVEVHTAQTQTHREDYNSTLRVVSALPIQHQDWMSGKEFKCKVNNK
DLPAPIERTISKPKGSVRAPQVYVLPPPEEEMTKKQVTLTCMVTDFMPEDIYVEWTNNGKTELNYKNTEP
VLDSDGSYFMYSKLRVEKKNWVERNSYSCSVVHEGLHNHHTTKSFSRTPGK
SEQ ID NO: 51 Human LILRB1(I142T/S155I) ectodomain fused to mIgG2aFc
GHLPKPTLWAEPGSVITQGSPVTLRCQGGQETQEYRLYREKKTALWITRIPQELVKKGQFPIPSITWEHA
GRYRCYYGSDTAGRSESSDPLELVVTGAYIKPTLSAQPSPVVNSGGNVTLQCDSQVAFDGFILCKEGED
EHPQCLNSQPHARGSSRAIFSVGPVSPSRRWWYRCYAYDSNSPYEWSLPSDLLELLVLGVSKKPSLSV
QPGPIVAPEETLTLQCGSDAGYNRFVLYKDGERDFLQLAGAQPQAGLSQANFTLGPVSRSYGGQYRCY
GAHNLSSEWSAPSDPLDILIAGQFYDRVSLSVQPGPTVASGENVTLLCQSQGWMQTFLLTKEGAADDPW
RLRSTYQSQKYQAEFPMGPVTSAHAGTYRCYGSQSSKPYLLTHPSDPLELVVSGPSGGPSSPTTGPTST
SGPEDQPLTPTGSDPQSGLGRHGGGGSEPRGPTIKPCPPCKCPAPNLLGGPSVFIFPPKIKDVLMISLSPI
VTCVVVDVSEDDPDVQISWFVNNVEVHTAQTQTHREDYNSTLRVVSALPIQHQDWMSGKEFKCKVNNK
DLPAPIERTISKPKGSVRAPQVYVLPPPEEEMTKKQVTLTCMVTDFMPEDIYVEWTNNGKTELNYKNTEP
VLDSDGSYFMYSKLRVEKKNWVERNSYSCSVVHEGLHNHHTTKSFSRTPGK
SEQ ID NO: 52 Rhesus monkey (Macaca mulatta) full length LILRB
MTPILMVLICLGLSLGSRTRVQAGTFPKPTLWAEPGSMISKGSPVTLRCQGSLPVQDYRLQREKKTASWV
RRIQQELVKKGYFPIASITSEHAGQYRCQYYSHSWWSEPSDPLELVVTGAYSKPTLSALPSPVVASGGNV
TLQCDSQVAGGFVLCKEGEDEHPQCLNSQPHTRGSSRAVFSVGPVSPSRRWSYRCYGYDSRSPYVWS
LPSDLLELLVPGVSKKPSLSVQPGPVVAPGDKLTLQCGSDAGYNRFALYKEGERDFLQRPGRQPQAGLS
QANFLLDPVRRSHGGQYRCSGAHNLSSEWSAPSDPLDILIAGQIRGRPSLLVQPGPTVVSGENVTLLCQS
SWQFHVFLLTQAGAADAHLHLRSMYKYPKYQAEFPMSPVTSAHAGTYRCYGSHSSDSYLLSIPSDPLEL
VVSGPSGGPSSPTTGPTSTCGPEDQPLTPTGSDPQSGLGRHLGVVTGVLVAFVLLLFLLLLLFLVLRHRR
QGKRWTSAQRKADFQHPAGAVEPEPRDRGLQRRSSPAANTQEENLYAAVKDTQPEDGVELDSRSPHD
EDPQAVTYARVKHSRPRREMASPPSPLSEEFLDTKDTQAAASEDPQDVTYAQLQSLTLRRETTEPPPSQ
EREPPVESSIYATLTIH
SEQ ID NO: 53 Cynomolgus monkey (Macaca fascicularis) full length LILRB
MHRGLLHPQS RAVGGDAMTP ILTVLICLGL SLGPRTHVQA GILPKPMLWA EPDRVITQGS
PVTLRCQGNL EALGYHLYRE RKSASWITSI RPELVRKGQF PIPSITWEDA GRYRCQYYSH
SWWSEHSDPL ELVVTGAYSK PTLSALPSPV VASGGNVTLQ CDSRVAFDGF ILCKEGEDEH
SQCLNSQPRT RGSSRAVFSV GPVSPSRRWS YRCYGYDSSF PYVWSLPSDL LELLVSGVSK
KPSLSVQPGP VVAPGDKVTL QCGSDAGYDR FVLYKEGERE FLQRPGRQPQ AGLSQANFTL
GPVSHSHGGQ YRCCGAHNLS SEWSAPSDPL DILISGQTHA RPSLSVQPGP TVASGENVTL
LCQSQGWMDT FFLTKEGAAD APLHLKSKRR SHIYQAEFPM GPVTSAHAGT YRCYGSFSSN
PYLLTHPSEP LELVVSGPSG SPSPPLTGPT PTPAGPEDQP LTPTGSAPQS GLGRHLGVVT
GILVAFVLLL FLLLLLFLVL RHQRQGKHWT SAQRKADFQH PAGAVEPEPR DRGFQRRSSP
AADTQEENLY AAVKDTQPED RVELDSRQSP HDEDPQAVTY AQVKHSGPRR EMTSPPSPLS
EEFLDTKDTQ AEEDRQRDTE AAASEDPQDV TYAQLQSLTL RREATEPPPT QEREPPAEPS
VYATLAIH
Reference Antibody 1 (anti-human LILRB1 Ab)
SEQ ID NO: 54 Reference Antibody 1 VH
DVQLQGSGPGLVKPSETLSLTCSVTGYSITSGYYWNWIRQFPGKKLEWMGYISYDGSNNYNPSLKNRITI
SRDTSKNQFSLKLNSVTAADTATYYCAHGYSYYYAMDAWGQGTSVTVSS
SEQ ID NO: 55 Reference Antibody 1 VL
DIQMTQSPSSLSASVGDRVTITCRTSQDISNYLNWYQQKPGKAVKLLISYTSRLHSGVPSRFSGSGSGTD
YTLTISSLQPEDFATYYCQQGNTLPTFGQGTKLEIK
Reference antibody 2 (anti-human LILRB2 Ab)
SEQ ID NO: 56 Reference antibody 2 Heavy chain including VH
EVQLQQWGAGLLKPSETLSLTCAVYGGSFSGYYWSWIRQPPGKGLEWIGEINHAGSTNYNPSLKSRVTI
SVDTSKNQFSLKLSSVTAADTAVYYCARLPTRWVTTRYFDLWGRGTLVTVSSASTKGPSVFPLAPCSRS
TSESTAALGCLVKDYFPEPVTVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDGSFFLYSRLTVDK
SRWQEGNVFSCSVMHEALHNHYTQKSLSLSLGK
SEQ ID NO: 57 Reference antibody 2 VL
ESVLTQPPSVSGAPGQRVTISCTGSSSNIGAGYDVHWYQQLPGTAPKLLIYGDSNRPSGVPDRFSVSKS
GASASLAITGLQAEDEADYYCQSFDNSLSAYVFGGGTQLTVLGQPKAAPSVTLFPPSSEELQANKATLVC
LISDFYPGAVTVAWKADSSPVKAGVETTTPSKQSNNKYAASSYLSLTPEQWKSHRSYSCQVTHEGSTVE
KTVAPTECS
Uniprot Reference sequences human LILRB1 to B5 and human LILRA1 to A6
SEQ ID NO: 59 Human LILRB1 Uniprot D9IDM8
MTPILTVLIC LGLSLGPRTH VQAGHLPKPT LWAEPGSVIT QGSPVTLRCQ GGQETQEYRL
YREKKTAPWI TRIPQELVKK GQFPIPSITW EHAGRYRCYY GSDTAGRSES SDPLELVVTG
AYIKPTLSAQ PSPVVNSGGN VTLQCDSQVA FDGFILCKEG EDEHPQCLNS QPHARGSSRA
IFSVGPVSPS RRWWYRCYAY DSNSPYEWSL PSDLLELLVL GVSKKPSLSV QPGPIVAPEE
TLTLQCGSDA GYNRFVLYKD GERDFLQLAG AQPQAGLSQA NFTLGPVSRS YGGQYRCYGA
HNLSSEWSAP SDPLDILIAG QFYDRVSLSV QPGPTVASGE NVTLLCQSQG WMQTFLLTKE
GAADDPWRLR STYQSQKYQA EFPMGPVTSA HAGTYRCYGS QSSKPYLLTH PSDPLELVVS
GPSGGPSSPT TGPTSTSGPE DQPLTPTGSD PQSGLGRHLG VVIGILVAVI LLLLLLLLLF LILRHRRQGK
HWTSTQRKAD FQHPAGAVGP EPTDRGLQWR SSPAADAQEE NLYAAVKHTQ PEDGVEMDTR
QSPHDEDPQA VTYAEVKHSR PRREMASPPS PLSGEFLDTK DRQAEEDRQM DTEAAASEAP
QDVTYAQLHS LTLRREATEP PPSQEGPSPA VPSIYATLAI H
SEQ ID NO: 60 Human LILRB2 Uniprot Q8N423-2
MTPIVTVLIC LGLSLGPRTR VQTGTIPKPT LWAEPDSVIT QGSPVTLSCQ GSLEAQEYRL YREKKSASWI
TRIRPELVKN GQFHIPSITW EHTGRYGCQY YSRARWSELS DPLVLVMTGA YPKPTLSAQP
SPVVTSGGRV TLQCESQVAF GGFILCKEGE DEHPQCLNSQ PHARGSSRAI FSVGPVSPNR
RWSHRCYGYD LNSPYVWSSP SDLLELLVPG VSKKPSLSVQ PGPVMAPGES LTLQCVSDVG
YDRFVLYKEG ERDLRQLPGR QPQAGLSQAN FTLGPVSRSY GGQYRCYGAH NLSSECSAPS
DPLDILITGQ IRGTPFISVQ PGPTVASGEN VTLLCQSWRQ FHTFLLTKAG AADAPLRLRS IHEYPKYQAE
FPMSPVTSAH AGTYRCYGSL NSDPYLLSHP SEPLELVVSG PSMGSSPPPT GPISTPGPED
QPLTPTGSDP QSGLGRHLGV VIGILVAVVL LLLLLLLLFL ILRHRRQGKH WTSTQRKADF
QHPAGAVGPE PTDRGLQWRS SPAADAQEEN LYAAVKDTQP EDGVEMDTRA AASEAPQDVT
YAQLHSLTLR RKATEPPPSQ EREPPAEPSI YATLAIH
SEQ ID NO: 61 Human LILRA3 Uniprot Q8N6C8-1
MTPILTVLIC LGLSLDPRTH VQAGPLPKPT LWAEPGSVIT QGSPVTLRCQ GSLETQEYHL
YREKKTALWI TRIPQELVKK GQFPILSITW EHAGRYCCIY GSHTAGLSES SDPLELVVTG AYSKPTLSAL
PSPVVTSGGN VTIQCDSQVA FDGFILCKEG EDEHPQCLNS HSHARGSSRA IFSVGPVSPS
RRWSYRCYGY DSRAPYVWSL PSDLLGLLVP GVSKKPSLSV QPGPVVAPGE KLTFQCGSDA
GYDRFVLYKE WGRDFLQRPG RQPQAGLSQA NFTLGPVSRS YGGQYTCSGA YNLSSEWSAP
SDPLDILITG QIRARPFLSV RPGPTVASGE NVTLLCQSQG GMHTFLLTKE GAADSPLRLK
SKRQSHKYQA EFPMSPVTSA HAGTYRCYGS LSSNPYLLTH PSDPLELVVS GAAETLSPPQ
NKSDSKAGE
SEQ ID NO: 62 Human LILRA1 Uniprot O75019-1
MTPIVTVLIC LRLSLGPRTH VQAGTLPKPT LWAEPGSVIT QGSPVTLWCQ GILETQEYRL
YREKKTAPWI TRIPQEIVKK GQFPIPSITW EHTGRYRCFY GSHTAGWSEP SDPLELVVTG AYIKPTLSAL
PSPVVTSGGN VTLHCVSQVA FGSFILCKEG EDEHPQCLNS QPRTHGWSRA IFSVGPVSPS
RRWSYRCYAY DSNSPHVWSL PSDLLELLVL GVSKKPSLSV QPGPIVAPGE SLTLQCVSDV
SYDRFVLYKE GERDFLQLPG PQPQAGLSQA NFTLGPVSRS YGGQYRCSGA YNLSSEWSAP
SDPLDILIAG QFRGRPFISV HPGPTVASGE NVTLLCQSWG PFHTFLLTKA GAADAPLRLR
SIHEYPKYQA EFPMSPVTSA HSGTYRCYGS LSSNPYLLSH PSDSLELMVS GAAETLSPPQ
NKSDSKAGAA NTLSPSQNKT ASHPQDYTVE NLIRMGIAGL VLVVLGILLF EAQHSQRSL
SEQ ID NO: 63 Human LILRA2 Uniprot Q8N149-1
MTPILTVLIC LGLSLGPRTH VQAGHLPKPT LWAEPGSVII QGSPVTLRCQ GSLQAEEYHL
YRENKSASWV RRIQEPGKNG QFPIPSITWE HAGRYHCQYY SHNHSSEYSD PLELVVTGAY
SKPTLSALPS PVVTLGGNVT LQCVSQVAFD GFILCKEGED EHPQRLNSHS HARGWSWAIF
SVGPVSPSRR WSYRCYAYDS NSPYVWSLPS DLLELLVPGV SKKPSLSVQP GPMVAPGESL
TLQCVSDVGY DRFVLYKEGE RDFLQRPGWQ PQAGLSQANF TLGPVSPSHG GQYRCYSAHN
LSSEWSAPSD PLDILITGQF YDRPSLSVQP VPTVAPGKNV TLLCQSRGQF HTFLLTKEGA
GHPPLHLRSE HQAQQNQAEF RMGPVTSAHV GTYRCYSSLS SNPYLLSLPS DPLELVVSEA
AETLSPSQNK TDSTTTSLGQ HPQDYTVENL IRMGVAGLVL VVLGILLFEA QHSQRSLQDA AGR
SEQ ID NO: 64 Human LILRB3 Uniprot O75022-1
MTPALTALLC LGLSLGPRTR VQAGPFPKPT LWAEPGSVIS WGSPVTIWCQ GSQEAQEYRL
HKEGSPEPLD RNNPLEPKNK ARFSIPSMTE HHAGRYRCHY YSSAGWSEPS DPLEMVMTGA
YSKPTLSALP SPVVASGGNM TLRCGSQKGY HHFVLMKEGE HQLPRTLDSQ QLHSRGFQAL
FPVGPVTPSH RWRFTCYYYY TNTPWVWSHP SDPLEILPSG VSRKPSLLTL QGPVLAPGQS
LTLQCGSDVG YNRFVLYKEG ERDFLQRPGQ QPQAGLSQAN FTLGPVSPSN GGQYRCYGAH
NLSSEWSAPS DPLNILMAGQ IYDTVSLSAQ PGPTVASGEN VTLLCQSWWQ FDTFLLTKEG
AAHPPLRLRS MYGAHKYQAE FPMSPVTSAH AGTYRCYGSY SSNPHLLSHP SEPLELVVSG
HSGGSSLPPT GPPSTPGLGR YLEVLIGVSV AFVLLLFLLL FLLLRRQRHS KHRTSDQRKT
DFQRPAGAAE TEPKDRGLLR RSSPAADVQE ENLYAAVKDT QSEDRVELDS QSPHDEDPQA
VTYAPVKHSS PRREMASPPS SLSGEFLDTK DRQVEEDRQM DTEAAASEAS QDVTYAQLHS
LTLRRKATEP PPSQEGEPPA EPSIYATLAI H
SEQ ID NO: 65 Human LILRB4 Uniprot Q8NHJ6 (isoform 1 is the canonical sequence)
MIPTFTALLC LGLSLGPRTH MQAGPLPKPT LWAEPGSVIS WGNSVTIWCQ GTLEAREYRL
DKEESPAPWD RQNPLEPKNK ARFSIPSMTE DYAGRYRCYY RSPVGWSQPS DPLELVMTGA
YSKPTLSALP SPLVTSGKSV TLLCQSRSPM DTFLLIKERA AHPLLHLRSE HGAQQHQAEF
PMSPVTSVHG GTYRCFSSHG FSHYLLSHPS DPLELIVSGS LEDPRPSPTR SVSTAAGPED
QPLMPTGSVP HSGLRRHWEV LIGVLVVSIL LLSLLLFLLL QHWRQGKHRT LAQRQADFQR
PPGAAEPEPK DGGLQRRSSP AADVQGENFC AAVKNTQPED GVEMDTRQSP HDEDPQAVTY
AKVKHSRPRR EMASPPSPLS GEFLDTKDRQ AEEDRQMDTE AAASEAPQDV TYAQLHSFTL
RQKATEPPPS QEGASPAEPS VYATLAIH
SEQ ID NO: 66 Human LILRB5 Uniprot O75023-1
MTLTLSVLIC LGLSVGPRTC VQAGTLPKPT LWAEPASVIA RGKPVTLWCQ GPLETEEYRL
DKEGLPWARK RQNPLEPGAK AKFHIPSTVY DSAGRYRCYY ETPAGWSEPS DPLELVATGF
YAEPTLLALP SPVVASGGNV TLQCDTLDGL LTFVLVEEEQ KLPRTLYSQK LPKGPSQALF
PVGPVTPSCR WRFRCYYYYR KNPQVWSNPS DLLEILVPGV SRKPSLLIPQ GSVVARGGSL
TLQCRSDVGY DIFVLYKEGE HDLVQGSGQQ PQAGLSQANF TLGPVSRSHG GQYRCYGAHN
LSPRWSAPSD PLDILIAGLI PDIPALSVQP GPKVASGENV TLLCQSWHQI DTFFLTKEGA AHPPLCLKSK
YQSYRHQAEF SMSPVTSAQG GTYRCYSAIR SYPYLLSSPS YPQELVVSGP SGDPSLSPTG
STPTPGPEDQ PLTPTGLDPQ SGLGRHLGVV TGVSVAFVLL LFLLLFLLLR HRHQSKHRTS
AHFYRPAGAA GPEPKDQGLQ KRASPVADIQ EEILNAAVKD TQPKDGVEMD APAAASEAPQ
DVTYAQLHSL TLRREATEPP PSQEREPPAE PSIYAPLAIH
SEQ ID NO: 67 Human LILRA4 Uniprot P59901-1
MTLILTSLLF FGLSLGPRTR VQAENLLKPI LWAEPGPVIT WHNPVTIWCQ GTLEAQGYRL
DKEGNSMSRH ILKTLESENK VKLSIPSMMW EHAGRYHCYY QSPAGWSEPS DPLELVVTAY
SRPTLSALPS PVVTSGVNVT LRCASRLGLG RFTLIEEGDH RLSWTLNSHQ HNHGKFQALF
PMGPLTFSNR GTFRCYGYEN NTPYVWSEPS DPLQLLVSGV SRKPSLLTLQ GPVVTPGENL
TLQCGSDVGY IRYTLYKEGA DGLPQRPGRQ PQAGLSQANF TLSPVSRSYG GQYRCYGAHN
VSSEWSAPSD PLDILIAGQI SDRPSLSVQP GPTVTSGEKV TLLCQSWDPM FTFLLTKEGA
AHPPLRLRSM YGAHKYQAEF PMSPVTSAHA GTYRCYGSRS SNPYLLSHPS EPLELVVSGA
TETLNPAQKK SDSKTAPHLQ DYTVENLIRM GVAGLVLLFL GILLFEAQHS QRSPPRCSQE
ANSRKDNAPF RVVEPWEQI
SEQ ID NO: 68 Human LILRA5 Uniprot A6NI73-1
MAPWSHPSAQ LQPVGGDAVS PALMVLLCLG LSLGPRTHVQ AGNLSKATLW AEPGSVISRG
NSVTIRCQGT LEAQEYRLVK EGSPEPWDTQ NPLEPKNKAR FSIPSMTEHH AGRYRCYYYS
PAGWSEPSDP LELVVTGFYN KPTLSALPSP VVTSGENVTL QCGSRLRFDR FILTEEGDHK
LSWTLDSQLT PSGQFQALFP VGPVTPSHRW MLRCYGSRRH ILQVWSEPSD LLEIPVSGAA
DNLSPSQNKS DSGTASHLQD YAVENLIRMG MAGLILVVLG ILIFQDWHSQ RSPQAAAGR
SEQ ID NO: 69 Human LILRA6 Uniprot Q6PI73-1
MTPALTALLC LGLSLGPRTR VQAGPFPKPT LWAEPGSVIS WGSPVTIWCQ GSLEAQEYQL
DKEGSPEPLD RNNPLEPKNK ARFSIPSMTQ HHAGRYRCHY YSSAGWSEPS DPLELVMTGF
YNKPTLSALP SPVVASGGNM TLRCGSQKGY HHFVLMKEGE HQLPRTLDSQ QLHSGGFQAL
FPVGPVTPSH RWRFTCYYYY TNTPRVWSHP SDPLEILPSG VSRKPSLLTL QGPVLAPGQS
LTLQCGSDVG YDRFVLYKEG ERDFLQRPGQ QPQAGLSQAN FTLGPVSPSH GGQYRCYGAH
NLSSEWSAPS DPLNILMAGQ IYDTVSLSAQ PGPTVASGEN VTLLCQSRGY FDTFLLTKEG
AAHPPLRLRS MYGAHKYQAE FPMSPVTSAH AGTYRCYGSY SSNPHLLSFP SEPLELMVSG
HSGGSSLPPT GPPSTPASHA KDYTVENLIR MGMAGLVLVF LGILLFEAQH SQRNPQDAAG R
Reference Antibody 3 (anti-human LILRB1 and LILRB2 Ab)
SEQ ID NO: 70 Reference Antibody 3 VH
QVQLVQSGAEVKKPGASVKVSCKASGYTFTDYYINWVRQAPGQGLEWMGNVNPNDGGTTYNQKFKGRVTMTTDT
STSTAYMELRSLRSDDTAVYYCARREIYFYGTIYYYAMDYWGQGTLVTVSS
SEQ ID NO: 71 Reference Antibody 3 VL
DIQLTQSPSFLSASVGDRVTITCRASESVDYYGNSFMYWYQQKPGKAPKLLIYFASNLESGVPSRFSGSGSGTEFTLTISSL
QPEDFATYYCQQNNEDPWTFGGGTKVEIK
Reference Antibody 4 (anti-human LILRB1 Ab)
SEQ ID NO: 72 Reference Antibody 4 VH
QVQLKESGPG LVAPSQSLSI TCTVSGFSLT SYGVSWVRQP PGKGLEWLGV IWGDGSTNYH
SALISRLSIS KDNSKSQVFL KLNSLQTDDT ATYYCAKPRW DDYAMDYWGQ GTSVTVSS
SEQ ID NO: 73 Reference Antibody 4 VL
DIQMTQTTSS LSASLGDRVT ISCRASQDIS NYLNWYQQKP DGTVKLLIYY TSRLHSGVPS
RFSGSGSGTD YSLTISNLEQ EDIATYFCQQ GNTLWTFGGG TKLEIK
Reference Antibody 5 (anti-human LILRB1 Ab)
SEQ ID NO: 74 Reference Antibody 5 VH
QVQLQQPGAE LVKPGASVRM SCKASGYTFT SYWVHWVKQR PGQGLEWIGV IDPSDSYTSY
NQNFKGKATL TVDTSSKTAY IHLSSLTSED SAVYFCARGE RYDGDYFAMD YWGQGTSVTV SS
SEQ ID NO: 75 Reference Antibody 5 VL
DIVMTQSPAS LSVSVGETVT ITCRASENIY SNLAWYQQKQ GKSPQLLVYA ATNLADGVPS
RFSGSRSGTQ YSLKINSLQS EDFGTYYCQH FWNTPRTFGG GTKLEIK
Reference Antibody 6 (anti-human LILRB1 Ab)
SEQ ID NO: 76 Reference Antibody 6 VH
QVQLQQSGAE LVKPGASVRL SCKASGYTFT AHTIHWVKQR SGQGLEWIGW LYPGSGSIKY
NEKFKDKATL TADKSSSTVY MELSRLTSED SAVYFCARHT NWDFDYWGQG TTLTVSS
SEQ ID NO: 77 Reference Antibody 6 VL
NIVLTQSPAS LAVSLGQRAT ISCKASQSVD YGGASYMNWY QQKPGQPPKL LIYAASNLES
GIPARFSGSG SGTDLTLNIH PVEEEDAAMY YCQQSNEEPW TFGGGTKLEI K
SEQ ID NO: 78 Epitope sequence in human LILRB1
AEFPMGPVTSAHAGT
SEQ ID NO: 79 Epitope sequence in human LILRB1
LTHPSDPLEL
SEQ ID NO: 80 Epitope sequence in human LILRB1
FVLYKDGERDF
SEQ ID NO: 81 Epitope sequence in human LILRB2
GYDRFVLYKEGERD (SEQ ID NO: 81) in human LILRB2, and sequence
SEQ ID NO: 82 Epitope sequence in human LILRA3
YDRFVLYKEWGRD
SEQ ID NO: 83 Epitope sequence in human LILRB1
SSEWSAPSDPLD
SEQ ID NO: 84 Epitope sequence in human LILRB2
ECSAPSDPLDI
SEQ ID NO: 85 Epitope sequence in human LILRA3
SEWSAPSDPLD
SEQ ID NO: 86 Epitope sequence in human LILRB2
LQCVSDVGYD
SEQ ID NO: 87 Epitope sequence in human LILRA3
FQCGSDAGYDRF
SEQ ID NO: 88 Epitope sequence in human LILRB1
FLLTKEGAADDPW
SEQ ID NO: 89 Epitope sequence in human LILRB2;
AADAPLRLRSIHEY
SEQ ID NO: 90 Epitope sequence in human LILRB1
RSYGGQYR
SEQ ID NO: 91 Epitope sequence in human LILRB2
PVSRSYGGQYRC in LILRB2,
SEQ ID NO: 92 Epitope sequence in human LILRB1
LDILIAGQFYD
SEQ ID NO: 93 Epitope sequence in human LILRB2
APSDPLDILI
SEQ ID NO: 94 Epitope sequence in human LILRA3
PSDPLDILI
SEQ ID NO: 95 LILRA4 recombinant, full-length ectodomain (LILRA4 24-446) (Uniprot P59901)
ENLLKPILWAEPGPVITWHNPVTIWCQGTLEAQGYRLDKEGNSMSRHILKTLESENKVKLSIPSMMWEHA
GRYHCYYQSPAGWSEPSDPLELVVTAYSRPTLSALPSPVVTSGVNVTLRCASRLGLGRFTLIEEGDHRLS
WTLNSHQHNHGKFQALFPMGPLTFSNRGTFRCYGYENNTPYVWSEPSDPLQLLVSGVSRKPSLLTLQG
PVVTPGENLTLQCGSDVGYIRYTLYKEGADGLPQRPGRQPQAGLSQANFTLSPVSRSYGGQYRCYGAH
NVSSEWSAPSDPLDILIAGQISDRPSLSVQPGPTVTSGEKVTLLCQSWDPMFTFLLTKEGAAHPPLRLRS
MYGAHKYQAEFPMSPVTSAHAGTYRCYGSRSSNPYLLSHPSEPLELVVSGATETLNPAQKKSDSKTAPH
LQDYTVEN
SEQ ID NO: 96 LILRA5 recombinant, full-length ectodomain (LILRA5 42-268) (Uniprot A6NI73)
GNLSKATLWAEPGSVISRGNSVTIRCQGTLEAQEYRLVKEGSPEPWDTQNPLEPKNKARFSIPSMTEHH
AGRYRCYYYSPAGWSEPSDPLELVVTGFYNKPTLSALPSPVVTSGENVTLQCGSRLRFDRFILTEEGDH
KLSWTLDSQLTPSGQFQALFPVGPVTPSHRWMLRCYGSRRHILQVWSEPSDLLEIPVSGAADNLSPSQN
KSDSGTASHLQDYAVENLIR
SEQ ID NO: 97 LILRA6 recombinant, full-length ectodomain (LILRA6 24-447) (Uniprot Q6PI73)
GPFPKPTLWAEPGSVISWGSPVTIWCQGSLEAQEYQLDKEGSPEPLDRNNPLEPKNKARFSIPSMTQHH
AGRYRCHYYSSAGWSEPSDPLELVMTGFYNKPTLSALPSPVVASGGNMTLRCGSQKGYHHFVLMKEGE
HQLPRTLDSQQLHSGGFQALFPVGPVTPSHRWRFTCYYYYTNTPRVWSHPSDPLEILPSGVSRKPSLLT
LQGPVLAPGQSLTLQCGSDVGYDRFVLYKEGERDFLQRPGQQPQAGLSQANFTLGPVSPSHGGQYRC
YGAHNLSSEWSAPSDPLNILMAGQIYDTVSLSAQPGPTVASGENVTLLCQSRGYFDTFLLTKEGAAHPPL
RLRSMYGAHKYQAEFPMSPVTSAHAGTYRCYGSYSSNPHLLSFPSEPLELMVSGHSGGSSLPPTGPPS
TPASHAKDYTVEN
SEQ ID NO: 98 LILRB1 recombinant, full-length ectodomain
GHLPKPTLWAEPGSVITQGSPVTLRCQGGQETQEYRLYREKKTALWITRIPQELVKKGQFPIPSITWEHA
GRYRCYYGSDTAGRSESSDPLELVVTGAYIKPTLSAQPSPVVNSGGNVILQCDSQVAFDGFSLCKEGED
EHPQCLNSQPHARGSSRAIFSVGPVSPSRRWWYRCYAYDSNSPYEWSLPSDLLELLVLGVSKKPSLSV
QPGPIVAPEETLTLQCGSDAGYNRFVLYKDGERDFLQLAGAQPQAGLSQANFTLGPVSRSYGGQYRCY
GAHNLSSEWSAPSDPLDILIAGQFYDRVSLSVQPGPTVASGENVTLLCQSQGWMQTFLLTKEGAADDPW
RLRSTYQSQKYQAEFPMGPVTSAHAGTYRCYGSQSSKPYLLTHPSDPLELVVSGPSGGPSSPTTGPTST
SGPEDQPLTPTGSDPQSGLGRHLGVVIGILVAVILLLLLLLLLFLILRHRRQGKHWTSTQRKADFQHPAGA
VGPEPTDRGLQWRSSPAADAQEENLYAAVKHTQPEDGVEMDTRSPHDEDPQAVTYAEVKHSRPRREM
ASPPSPLSGEFLDTKDRQAEEDRQMDTEAAASEAPQDVTYAQLHSLTLRREATEPPPSQEGPSPAVPSI
YATLAIH
SEQ ID NO: 99 LILRB2 recombinant, full-length ectodomain (LILRB2 22-458) (Uniprot Q8N423.4)
QTGTIPKPTLWAEPDSVITQGSPVTLSCQGSLEAQEYRLYREKKSASWITRIRPELVKNGQFHIPSITWEH
TGRYGCQYYSRARWSELSDPLVLVMTGAYPKPTLSAQPSPVVTSGGRVTLQCESQVAFGGFILCKEGE
DEHPQCLNSQPHARGSSRAIFSVGPVSPNRRWSHRCYGYDLNSPYVWSSPSDLLELLVPGVSKKPSLS
VQPGPVMAPGESLTLQCVSDVGYDRFVLYKEGERDLRQLPGRQPQAGLSQANFTLGPVSRSYGGQYR
CYGAHNLSSECSAPSDPLDILITGQIRGTPFISVQPGPTVASGENVTLLCQSWRQFHTFLLTKAGAADAPL
RLRSIHEYPKYQAEFPMSPVTSAHAGTYRCYGSLNSDPYLLSHPSEPLELVVSGPSMGSSPPPTGPISTP
GPEDQPLTPTGSDPQSGLGRHL
SEQ ID NO: 100 LILRB3 recombinant, full-length ectodomain (LILRB3 24-443) (Uniprot aab68668)
GPFPKPTLWAEPGSVISWGSPVTIWCQGSQEAQEYRLHKEGSPEPLDRNNPLEPKNKARFSIPSMTEHH
AGRYRCHYYSSAGWSEPSDPLEMVMTGAYSKPTLSALPSPVVASGGNMTLRCGSQKGYHHFVLMKEG
EHQLPRTLDSQQLHSRGFQALFPVGPVTPSHRWRFTCYYYYTNTPWVWSHPSDPLEILPSGVSRKPSLL
TLQGPVLAPGQSLTLQCGSDVGYNRFVLYKEGERDFLQRPGQQPQAGLSQANFTLGPVSPSNGGQYR
CYGAHNLSSEWSAPSDPLNILMAGQIYDTVSLSAQPGPTVASGENVTLLCQSWWQFDTFLLTKEGAAHP
PLRLRSMYGAHKYQAEFPMSPVTSAHAGTYRCYGSYSSNPHLLSHPSEPLELVVSGHSGGSSLPPTGPP
STPGLGRYLE
SEQ ID NO: 101 LILRB4 recombinant, full-length ectodomain (LILRB4 17-257) (Uniprot AAH26309)
PRTHMQAGPLPKPTLWAEPGSVISWGNSVTIWCQGTLEAREYRLDKEESPAPWDRQNPLEPKNKARFSI
PSMTEDYAGRYRCYYRSPVGWSQPSDPLELVMTGAYSKPTLSALPSPLVTSGKSVTLLCQSRSPMDTFL
LIKERAAHPLLHLRSEHGAQQHQAEFPMSPVTSVHGGTYRCFSSHGFSHYLLSHPSDPLELIVSGSLEDP
RPSPTRSVSTAAGPEDQPLMPTGSVPHSGLRRH
SEQ ID NO: 102 LILRB5 recombinant, full-length ectodomain (LILRB5 18-456) (Uniprot O75023)
RTCVQAGTLPKPTLWAEPASVIARGKPVTLWCQGPLETEEYRLDKEGLPWARKRQNPLEPGAKAKFHIP
STVYDSAGRYRCYYETPAGWSEPSDPLELVATGFYAEPTLLALPSPVVASGGNVTLQCDTLDGLLTFVLV
EEEQKLPRTLYSQKLPKGPSQALFPVGPVTPSCRWRFRCYYYYRKNPQVWSNPSDLLEILVPGVSRKPS
LLIPQGSVVARGGSLTLQCRSDVGYDIFVLYKEGEHDLVQGSGQQPQAGLSQANFTLGPVSRSHGGQY
RCYGAHNLSPRWSAPSDPLDILIAGLIPDIPALSVQPGPKVASGENVTLLCQSWHQIDTFFLTKEGAAHPP
LCLKSKYQSYRHQAEFSMSPVTSAQGGTYRCYSAIRSYPYLLSSPSYPQELVVSGPSGDPSLSPTGSTP
TPGPEDQPLTPTGLDPQSGLGRH
Reference Antibody 7 (anti-human LILRB2 Ab)
SEQ ID NO: 103 Reference Antibody 7 VH
QITLKESGPTLVKPTQTLTLTCTFSGFSLNTYAMGVSWIRQPPGKALEWLASIWWNGNKYNNPSLKSRLT
VTKDTSKNQVVLTMTNMDPVDTATYYCAHSRIIRFTDYVMDAWGQGTLVTVSSASTKGPSVFPLAPCSR
STSESTAALGCLVKDYFPEPVTVSWNSGALTSGVHTFPAVLQSSGLYSLSSVVTVPSSSLGTKTYTCNVD
HKPSNTKVDKRVESKYGPPCPSCPAPEFLGGPSVFLFPPKPKDTLMISRTPEVTCVVVDVSQEDPEVQF
NWYVDGVEVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFFLYSRLTVD
KSRWQEGNVFSCSVMHEALHNHYTQKSLSLSLGK
SEQ ID NO: 104 Reference Antibody 7 VL
DIQMTQSPSSLSTSVGDRVTITCRASEDIYNDLAWYQQKPGKAPKLLIYNANSLHTGVASRFSGSGSGTD
FTFTISSLQPEDVATYFCQQYYDYPLTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNR
GEC
SEQ ID NO: 105 HMGB1 peptide
FKDPNAPKRLPSAFFLFCSE

1. An antigen-binding protein capable of binding specifically to human LILRB1 and to human LILRB2, wherein the antigen-binding protein does not block the interaction of human LILRB1 with HLA-G tetramer and/or does not block the interaction of human LILRB2 with HLA-G tetramer, and the antigen-binding protein is capable of reprogramming macrophage.

2. An antigen-binding protein of claim 1 that is capable of reprogramming fully differentiated macrophage to an anti-tumoural (pro-inflammatory) phenotype.

3. An antigen-binding protein, of claim 1 or claim 2, wherein reprogramming is indicated/detected by induction of a marker of macrophage reprogramming.

4. An antigen-binding protein, of any preceding claim, wherein reprogramming is indicated/detected by release of a pro-inflammatory cytokine from the macrophage following exposure of the macrophage to the antigen-binding protein and LPS stimulation.

5. An antigen-binding protein, of any preceding claim, wherein reprogramming is indicated/detected by release of a pro-inflammatory cytokine TNF alpha and/or GM-CSF from macrophages following exposure to antigen-binding protein and LPS stimulation.

6. An antigen-binding protein, of any preceding claim, wherein the antigen-binding protein has one or more property selected from:
(a) stimulates the production of GM-CSF and/or TNFalpha on LPS stimulation of iPS-derived macrophage and/or primary-monocyte-derived macrophage,
(b) stimulates the production of GM-CSF and/or TNFalpha on LPS stimulation of primary-monocyte-derived macrophage expressing LILRB1 and LILRB2, and,
(c) stimulates the production of GM-CSF and/or TNFalpha on LPS stimulation of human macrophages expressing LILRB1 and LILRB2.

7. An antigen-binding protein of any preceding claim, wherein the antigen-binding protein has one or more property selected from the ability to:
(a) induce phagocytosis,
(b) induce phagocytosis in the absence of a second signal,
(c) induce phagocytosis in the absence of a second antibody (e,g, an anti-CD47 antibody or an anti-EGFR antibody),
(d) induce phagocytosis in the absence of second, opsonizing antibody (e,g, an anti-CD47 antibody, or an anti-EGFR antibody),
(e) induce phagocytosis of cancer cells in the absence of second, opsonizing antibody (e,g, a tumour binding antibody), and
(f) induce phagocytosis of MHC Class I positive and/or MHC Class I negative cancer cells.

8. An antigen-binding protein of any preceding claim, capable of binding specifically to:
(a) human LILRB1, human LILRB2 and human LILRA3;
(b) human LILRB1, human LILRB2, human LILRA3 and human LILRA1;
(c) human LILRB1, human LILRB2, human LILRB3, human LILRA3, human LILRA4, and human LILRA6; and/or,
(d) human LILRB1, human LILRB2, human LILRB3, human LILRA1, human LILRA3, human LILRA4, and human LILRA6.

9. An antigen-binding protein of any preceding clause that does not bind to human LILRB4, human LILRB5, human LILRA2 or human LILRA5.

10. An antigen-binding protein of any preceding clause wherein binding is assessed by flow cytometry or ELISA.

11. An antigen-binding protein of any preceding claim, capable of binding specifically to a rhesus monkey and/or cynomolgus monkey homologue of a human LILRB1 or LIRB2 ectodomain.

12. An antigen-binding protein according to any one of the preceding claims that binds an epitope common to:
(a) human LILRB1, LILRB2 and LILRA3;
(b) human LILRB1, LILRB2, LILRA3 and LILRA1;
(c) human LILRB1, LILRB2, LILRB3, LILRA3, LILRA4, and LILRA6; and/or
(d) human LILRB1, LILRB2, LILRB3, LILRA1, LILRA3, LILRA4, and LILRA6.

13. An antigen-binding protein according to any one of the preceding claims that binds an epitope common to:
(a) human LILRB1, LILRB2 and LILRA3;
(b) human LILRB1, LILRB2, LILRA3 and LILRA1;
(c) human LILRB1, LILRB2, LILRB3, LILRA3, LILRA4, and LILRA6; and/or
(d) human LILRB1, LILRB2, LILRB3, LILRA1, LILRA3, LILRA4, and LILRA6.

14. An antigen-binding protein according to any one of the preceding clauses wherein the epitope is formed by:
(a) the sequence AEFPMGPVTSAHAGT (SEQ ID NO: 78) of human LILRB1;
(b) the sequence AEFPMGPVTSAHAGT (SEQ ID NO:78) and the sequence LTHPSDPLEL (SEQ ID NO: 79) of human LILRB1;
wherein the epitope is mapped using hydrogen-deuterium exchange (HDX) mass spectrometry method.

15. An antigen-binding protein according to any one of the preceding clauses wherein the epitope is formed by:
(a) sequence FVLYKDGERDF (SEQ ID NO: 80) in human LILRB1, sequence GYDRFVLYKEGERD (SEQ ID NO: 81) in human LILRB2, and sequence YDRFVLYKEWGRD (SEQ ID NO: 82) in human LILRA3;
(b) sequence SSEWSAPSDPLD (SEQ ID NO: 83) in LILRB1, sequence ECSAPSDPLDI (SEQ ID NO: 84) in LILRB2, and sequence SEWSAPSDPLD (SEQ ID NO: 85) in LILRA3;
(c) sequence LQCVSDVGYD (SEQ ID NO: 86) in LILRB2 and sequence FQCGSDAGYDRF (SEQ ID NO: 87) in LILRA3;
(d) sequence FLLTKEGAADDPW (SEQ ID NO: 88) in LILRB1 and sequence AADAPLRLRSIHEY (SEQ ID NO: 89) in LILRB2;
(e) sequence RSYGGQYR (SEQ ID NO: 90) in LILRB1 and sequence PVSRSYGGQYRC (SEQ ID NO: 91) in LILRB2; or,
(f) sequence LDILIAGQFYD (SEQ ID NO: 92) in LILRB1, sequence APSDPLDILI (SEQ ID NO: 93) in LILRB2, and sequence PSDPLDILI (SEQ ID NO: 94) in LILRA3);
wherein the epitope is mapped using binding to peptide microarrays.

16. An antigen-binding protein according to any one of the preceding claims, wherein the antigen-binding protein is an antibody or an antigen-binding fragment thereof.

17. An antigen-binding protein according to any one of the preceding claims, wherein the antigen-binding protein is a human antibody or an antigen-binding fragment thereof.

18. An antigen-binding protein according to any one of the preceding claims, wherein the antigen-binding protein is a monoclonal antibody, such as a human monoclonal antibody.

19. An antigen-binding protein according to any one of the preceding claims, wherein the antigen-binding protein comprises an Fc.

20. An antigen-binding protein according to any one of the preceding claims, comprising the six CDRs (HCDR1, HCRD2, HCDR3, LCDR1, LCDR2 and LCDR3, respectively) of an antibody selected from:
(a) Antibody 1 of SEQ ID NO: 1, SEQ ID NO: 2, SEQ ID NO: 3, SEQ ID NO: 4, SEQ ID NO: 5 and SEQ ID NO: 6;
(b) Antibody 2 of SEQ ID NO: 9, SEQ ID NO: 10, SEQ ID NO: 11, SEQ ID NO: 12, SEQ ID NO: 13 and SEQ ID NO: 14;
(c) Antibody 3 of SEQ ID NO: 17, SEQ ID NO: 18, SEQ ID NO: 19, SEQ ID NO: 20, SEQ ID NO: 21 and SEQ ID NO: 22;
(d) Antibody 4 of SEQ ID NO: 25, SEQ ID NO: 26, SEQ ID NO: 27, SEQ ID NO: 28, SEQ ID NO: 29 and SEQ ID NO: 30; and
(e) Antibody 5 of SEQ ID NO: 33, SEQ ID NO: 34, SEQ ID NO: 35, SEQ ID NO: 36, SEQ ID NO: 37 and SEQ ID NO: 38;
wherein the sequences are defined using Kabat nomenclature.

21. An antigen-binding protein according to any one of the preceding claims, comprising a VH and VL, respectively, of an antibody selected from:
(a) Antibody 1 of SEQ ID NO: 7 and SEQ ID NO: 8;
(b) Antibody 2 of SEQ ID NO: 15 and SEQ ID NO: 16;
(c) Antibody 3 of SEQ ID NO: 23 and SEQ ID NO: 24;
(d) Antibody 4 of SEQ ID NO: 31 and SEQ ID NO: 32; and
(e) Antibody 5 of SEQ ID NO: 39 and SEQ ID NO: 40;
wherein the sequences are defined using Kabat nomenclature.

22. An antigen-binding protein, such as a human antibody or an antigen-binding fragment thereof, that is capable of competing for binding to human LILRB1, human LILRB2 and/or human LILRA3 with an antigen-binding protein, such as an antibody or an antigen-binding fragment thereof, according to any one of the preceding claims.

23. An antigen-binding protein, such as a human antibody or an antigen-binding fragment thereof, according to claim 22, wherein competing for binding is assessed using a competition assay selected from a cell-based binding assay, a cell-free binding assay, an immunoassay, ELISA, HTRF, flow cytometry, fluorescent microvolume assay technology (FMAT) assay, Mirrorball, high content imaging based fluorescent immunoassays, radioligand binding assays, bio-layer interferometry (BLI), surface plasmon resonance (SPR) and thermal shift assays.

24. A composition comprising an antigen-binding protein according to any one of claims 1 to 23 and a diluent.

25. An antigen-binding protein according to any one of claims 1 to 23 or a composition according to claim 24:
(a) for use as a medicament;
(b for use as a medicament for the treatment of cancer;
(c) for use in the treatment of a cancer;
(d) for use in the manufacture of a medicament for the treatment of a cancer;
wherein optionally the cancer of (b), (c) or (d) is selected from:
(i) Acute Myeloid Leukemia (AML), Bladder Urothelial Carcinoma (BLCA), Brain Lower Grade Glioma (LGG), Breast invasive carcinoma (BRCA), Esophageal carcinoma (ESCA), Glioblastoma multiforme (GBM), Head and Neck squamous cell carcinoma (HNSC), Kidney renal clear cell carcinoma (KIRC), Kidney renal papillary cell carcinoma (KIRP), Liver hepatocellular carcinoma (LIHC), Lung adenocarcinoma (LUAD), Lung squamous cell carcinoma (LUSC), Pancreatic adenocarcinoma (PAAD), Sarcoma (SARC), Skin Cutaneous Melanoma (SKCM), Stomach adenocarcinoma (STAD), Testicular Gemm Cell Tumours (TGCT), Thymoma (THYM), Thyroid carcinoma (THCA), Uterine Carcinosarcoma (UCS), Uterine Corpus Endometrial Carcinoma (UCEC), Uveal Melanoma (UVM), colorectal cancer, prostate cancer, pediatric cancers, lymphomas and leukemias such as DLBCL, NHL, multiple myeloma, Hodgkins lymphoma;
(ii) cancer positive for LILRB1 or LILRB2 or positive for both LILRB1 and LILRB2;
(iii) cancer positive for immunosuppressive macrophages (as measured by CD163 or CD68 positivity) and/or tumour infiltrating T cells;
(iv) cancer with increased or decreased expression of classical or non-classical MHC Class I;
(v) cancer positive for one or more of LILRB1, LILRB2, LILRB3, LILRA1, LILRA3, LILRA4, and LILRA6;
(e) for use as a medicament for the treatment of an immunosuppressive disease;
(f) for use in the treatment of an immunosuppressive disease; or
(g) for use in the manufacture of a medicament for the treatment of an immunosuppressive disease.

26. A method of treatment of a cancer, or of treatment of an immunosuppressive disease, comprising administration of an antigen-binding protein of any one of claims 1 to 23, or a composition according to claim 24, to a subject.

27. An isolated recombinant DNA or RNA sequence comprising a sequence encoding an antigen-binding protein of any one of claims 1 to 23.

28. An isolated recombinant DNA sequence of claim 27 which is a vector, optionally wherein the vector is an expression vector.

29. An isolated recombinant DNA sequence of claim 27 or 28 encoding an antigen-binding protein of any one of claims 1 to 23 under control of a promoter.

30. A host cell comprising a DNA or RNA sequence according to any one of claims 27 to 29, optionally wherein the host cell is capable of expressing an antigen-binding protein of any one of claims 1 to 23.

31. A method of making an isolated antigen-binding protein of any one of claims 1 to 23 comprising culturing a host cell of claim 30 in conditions suitable for expression of the isolated antibody or antigen-binding fragment thereof.

32. A method of identifying an antigen-binding protein, of any one of claims 1 to 23 comprising:
(a) providing one or more antigen-binding protein capable of binding to:
(i) human LILRB1, LILRB2 and/or LILRA3;
(ii) human LILRB1, LILRB2 and LILRA3 protein;
(iii) human LILRB1, LILRB2, LILRA1 and LILRA3 protein;
(iv) human LILRB1, LILRB2, LILRB3, LILRA3, LILRA4, and LILRA6 protein; and/or
(v) human LILRB1, LILRB2, LILRB3, LILRA1, LILRA3, LILRA4, and LILRA6 protein;
and performing one or more assessment selected from (b), (c) and (d):
(b) assessing the ability of the one or more antigen-binding protein to modulate one or more biological activity/phenotype of a human macrophage, such as to promote phagocytosis and/or pro-inflammatory cytokine release (such as TNFalpha or GM-SCF), or expression of macrophage activation markers (such as HLA-DR and/or CD80):
(c) assessing the ability of the one or more antigen-binding protein to block binding of LILRB1 and/or LILRB2 to cells expressing a ligand of LILRB1 and/or LILRB2, e.g., HLA-G, and selecting one or more antibodies that bind to LILRB1, LILRB2 and LILRA3 and do not block binding of LILRB1 and/or LILRB2 to target cells expressing a ligand of LILRB1 and/or LILRB2, for example HLA-G;
(d) assessing the ability of the one more antigen-binding protein to block binding of ligand (e.g. HLA-G) to cells expressing LILRB1 and/or LILRB2, and selecting one or more antibodies that do not block binding of a ligand of LILRB1 and/or LILRB2, for example HLA-G, to a cell expressing LILRB1 and/or LILRB2; and
(e) selecting one or more antigen-binding protein capable of binding specifically to human LILRB1 and to human LILRB2, wherein the antigen-binding protein does not block the interaction of human LILRB1 with HLA-G tetramer and is capable of reprogramming macrophage, and optionally,
(f) formulating the one or more antigen-binding protein into a composition with one or more excipient.

33. A method of identifying an antibody or antigen-binding fragment thereof, of any one of claims 1 to 23 comprising:
(a) providing one or more antibody or antigen-binding fragment thereof capable of binding to:
(i) human LILRB1, LILRB2 and/or LILRA3 protein;
(ii) human LILRB1, LILRB2 and LILRA3 protein;
(iii) human LILRB1, LILRB2, LILRA1 and LILRA3 protein;
(iv) human LILRB1, LILRB2, LILRB3, LILRA3, LILRA4, and LILRA6 protein; and/or,
(v) human LILRB1, LILRB2, LILRB3, LILRA1, LILRA3, LILRA4, and LILRA6 protein;
and performing one or more assessment selected from (b), (c) and (d):
(b) assessing the ability of the one more antibody or antigen-binding fragment thereof to block binding of LILRB1 and/or LILRB2 to cells expressing a ligand of LILRB1 and/or LILRB2, e.g., HLA-G, and selecting one or more antibodies that bind to LILRB1, LILRB2 and LILRA3 and do not block binding of LILRB1 and/or LILRB2 to target cells expressing a ligand of LILRB1 and/or LILRB2, for example HLA-G;
(c) assessing the ability of the one more antibody or antigen-binding fragment thereof to block binding of ligand (e.g. HLA-G) to cells expressing LILRB1 and/or LILRB2, and selecting one or more antibodies that do not block binding of a ligand of LILRB1 and/or LILRB2, for example HLA-G, to a cell expressing LILRB1 and/or LILRB2;
(d) assessing the ability of the one or more antibody or antigen-binding fragment thereof to modulate one or more biological activity/phenotype of a human macrophage, e.g., to promote phagocytosis and/or pro-inflammatory cytokine release (such as TNFalpha or GM-SCF), or expression of macrophage activation markers (such as HLA-DR and/or CD80).


SEQUENCE LISTING (105 sequences)

SEQ ID NO: 1 - protein, 5 aa, Homo sapiens
DYYMS

SEQ ID NO: 2 - protein, 17 aa, Homo sapiens
YISSSGSIKKYADSVKG

SEQ ID NO: 3 - protein, 7 aa, Homo sapiens
TNWHFDY

SEQ ID NO: 4 - protein, 12 aa, Homo sapiens
RASQSVSSSYLA

SEQ ID NO: 5 - protein, 7 aa, Homo sapiens
GASTRAT

SEQ ID NO: 6 - protein, 9 aa, Homo sapiens
QQYYSTPFT

SEQ ID NO: 7 - protein, 116 aa, Homo sapiens
QVQLVESGGGLVKPGGSLRLSCAASGFTFSDYYMSWIRQAPGKGLEWVSYISSSGSIKKYADSVKGRFTISRDNAKNSLYLQMNSLRGEDTAVYYCARTNWHFDYWGQGTLVTVSS

SEQ ID NO: 8 - protein, 108 aa, Homo sapiens
EIVLTQSPGTLSLSPGERATLSCRASQSVSSSYLAWYQQKPGQAPRLLIFGASTRATGIPDRFSGSGSGTDFTLTISRLEPEDVAVYYCQQYYSTPFTFGPGTKVDIK

SEQ ID NO: 9 - protein, 5 aa, Homo sapiens
DYYMS

SEQ ID NO: 10 - protein, 17 aa, Homo sapiens
YISPSGSTIFYADSVKG

SEQ ID NO: 11 - protein, 8 aa, Homo sapiens
DRVRLFDY

SEQ ID NO: 12 - protein, 12 aa, Homo sapiens
RASQSVSSSYLA

SEQ ID NO: 13 - protein, 7 aa, Homo sapiens
GASTRAT

SEQ ID NO: 14 - protein, 9 aa, Homo sapiens
QQRSNWPIT

SEQ ID NO: 15 - protein, 117 aa, Homo sapiens
QVQLVESGGDLVKPGGSLRLSCAASGFTFRDYYMSWIRQAPGKGLEWVSYISPSGSTIFYADSVKGRFTISRDNAKNSLYLQMNSLRAEDTAVYYCAKDRVRLFDYWGQGTLVTVSS

SEQ ID NO: 16 - protein, 108 aa, Homo sapiens
EIVLTQSPATLSLSPGERATLSCRASQSVSSSYLAWYQQKPGQAPRLLIYGASTRATGIPARFSGSGSGTDFTLTISRLEPEDFAVYYCQQRSNWPITFGQGTRLEIK

SEQ ID NO: 17 - protein, 5 aa, Homo sapiens
SYGIS

SEQ ID NO: 18 - protein, 17 aa, Homo sapiens
WISAYNGNTNYAQKLQG

SEQ ID NO: 19 - protein, 11 aa, Homo sapiens
SYSGSHWWFDP

SEQ ID NO: 20 - protein, 17 aa, Homo sapiens
KSSQSVLYSSNNNNYLA

SEQ ID NO: 21 - protein, 7 aa, Homo sapiens
WAFSRES

SEQ ID NO: 22 - protein, 9 aa, Homo sapiens
QQYYSTPPT

SEQ ID NO: 23 - protein, 120 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARSYSGSHWWFDPWGQGTLVTVSS

SEQ ID NO: 24 - protein, 113 aa, Homo sapiens
DIVMTQSPDSLAVSLGERATINCKSSQSVLYSSNNNNYLAWYQRKPGQPPKLLINWAFSRESGVPDRFSGSGSGTDFTLTISSLQAEDVAVYYCQQYYSTPPTFGQGTKVEIK

SEQ ID NO: 25 - skipped

SEQ ID NO: 26 - protein, 17 aa, Homo sapiens
WISAYNGNTNYAQKLQG

SEQ ID NO: 27 - protein, 11 aa, Homo sapiens
SYSGSYWWFDP

SEQ ID NO: 28 - protein, 17 aa, Homo sapiens
KSSQSVLYSSNNNNYLA

SEQ ID NO: 29 - protein, 7 aa, Homo sapiens
WASTRES

SEQ ID NO: 30 - protein, 9 aa, Homo sapiens
QQYYSTPLT

SEQ ID NO: 31 - protein, 120 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARSYSGSYWWFDPWGQGTLVTVSS

SEQ ID NO: 32 - protein, 113 aa, Homo sapiens
DIVMTQSPDSLAVSLGERATINCKSSQSVLYSSNNNNYLAWYQQKPGQPPKLLINWASTRESGVPDRFSGSGSGTDFTLTISSLQAEDVAVYYCQQYYSTPLTFGGGTKVEIK

SEQ ID NO: 33 - protein, 5 aa, Homo sapiens
DYYMT

SEQ ID NO: 34 - protein, 17 aa, Homo sapiens
YISISGITRYYADSVKG

SEQ ID NO: 35 - protein, 8 aa, Homo sapiens
DQTGYFDY

SEQ ID NO: 36 - protein, 11 aa, Homo sapiens
RASQSFSSNLA

SEQ ID NO: 37 - protein, 7 aa, Homo sapiens
GASTRAT

SEQ ID NO: 38 - protein, 9 aa, Homo sapiens
QQYINWPHT

SEQ ID NO: 39 - protein, 117 aa, Homo sapiens
QVQLVESGGGLVKPGGSLRLSCAASGFTFSDYYMTWIRQAPGKGLEWVSYISISGITRYYADSVKGRFTISRDNAKNSLYLQMNSLRAEDTAVYYCARDQTGYFDYWGQGTLVTVSS

SEQ ID NO: 40 - protein, 107 aa, Homo sapiens
DIVMTQSPATLSVSPGEKTTLSCRASQSFSSNLAWYQQKPGQAPRLLIYGASTRATGIPARFSGSGSGTEFTLTISSLQSEDFAVYYCQQYINWPHTFGQGTKVEIK

SEQ ID NO: 41 - protein, 673 aa, Synthetic construct
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHGGGGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 42 - protein, 435 aa, synthetic construct
PKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSGGGGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 43 - protein, 643 aa, Synthetic construct
SRTRVQAGTFPKPTLWAEPGSMISKGSPVTLRCQGSLPVQDYRLQREKKTASWVRRIQQELVKKGYFPIASITSEHAGQYRCQYYSHSWWSEPSDPLELVVTGAYSKPTLSALPSPVVASGGNVTLQCDSQVAGGFVLCKEGEDEHPQCLNSQPHTRGSSRAVFSVGPVSPSRRWSYRCYGYDSRSPYVWSLPSDLLELLVPGVSKKPSLSVQPGPVVAPGDKLTLQCGSDAGYNRFALYKEGERDFLQRPGRQPQAGLSQANFLLDPVRRSHGGQYRCSGAHNLSSEWSAPSDPLDILIAGQIRGRPSLLVQPGPTVVSGENVTLLCQSSWQFHVFLLTQAGAADAHLHLRSMYKYPKYQAEFPMSPVTSAHAGTYRCYGSHSSDSYLLSIPSDPLELVVSGPGGGGGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 44 - protein, 695 aa, Synthetic construct
MGWSCIILFLVATATGVHSGILPKPMLWAEPDRVITQGSPVTLRCQGNLEALGYHLYRERKSASWITLIRPELVKKGQFPIPSITWEDAGRYRCQYYSHSWWSEPSDPLELVVTGAYRKPTLSALPSPVVASGGNVTLQCDSQVPFDGFILCKEGEDEHPQRLNCQSHARGWSRAVFSVGPVSPSRRWSYRCYGYDSRSPYVWSLPSDLLELLVPGVSKKPSLSVQPGPVVAPGEKLTLQCGSDAGYDRFALYKEGEGEFLQRPGRQPQAGLSQANFPLGPVSRSHGGQYRCSGAHNLSSEWSAPSDPLDILISGQFHDVVSLSVQPGPTVASGENVTLLCQSRAQFHTFLLTREGAAHPPLHLRSEHQAQQYQAEFPMSPVTSAHAGTYRCYGSLSSNPYLLTHPSEPLELVVSGPSRSPSPPLTGPTPTPAGPEDQSLNPTGSDPQSGLGRHLGVGGGGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 45 - protein, 650 aa, Homo sapiens
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 46 - protein, 598 aa, Homo sapiens
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 47 - protein, 445 aa, Homo sapiens
PRTHVQAGTLPKPTLWAEPGSVITQGSPVTLWCQGILETQEYRLYREKKTAPWITRIPQEIVKKGQFPIPSITWEHTGRYRCFYGSHTAGWSEPSDPLELVVTGAYIKPTLSALPSPVVTSGGNVTLHCVSQVAFGSFILCKEGEDEHPQCLNSQPRTHGWSRAIFSVGPVSPSRRWSYRCYAYDSNSPHVWSLPSDLLELLVLGVSKKPSLSVQPGPIVAPGESLTLQCVSDVSYDRFVLYKEGERDFLQLPGPQPQAGLSQANFTLGPVSRSYGGQYRCSGAYNLSSEWSAPSDPLDILIAGQFRGRPFISVHPGPTVASGENVTLLCQSWGPFHTFLLTKAGAADAPLRLRSIHEYPKYQAEFPMSPVTSAHSGTYRCYGSLSSNPYLLSHPSDSLELMVSGAAETLSPPQNKSDSKAGAANTLSPSQNKTASHPQDYTVEN

SEQ ID NO: 48 - protein, 426 aa, Homo sapiens
GHLPKPTLWAEPGSVIIQGSPVTLRCQGSLQAEEYHLYRENKSASWVRRIQEPGKNGQFPIPSITWEHAGRYHCQYYSHNHSSEYSDPLELVVTGAYSKPTLSALPSPVVTLGGNVTLQCVSQVAFDGFILCKEGEDEHPQRLNSHSHARGWSWAIFSVGPVSPSRRWSYRCYAYDSNSPYVWSLPSDLLELLVPGVSKKPSLSVQPGPMVAPGESLTLQCVSDVGYDRFVLYKEGERDFLQRPGWQPQAGLSQANFTLGPVSPSHGGQYRCYSAHNLSSEWSAPSDPLDILITGQFYDRPSLSVQPVPTVAPGKNVTLLCQSRGQFHTFLLTKEGAGHPPLHLRSEHQAQQNQAEFRMGPVTSAHVGTYRCYSSLSSNPYLLSLPSDPLELVVSEAAETLSPSQNKTDSTTTSLGQHPQDYTVEN

SEQ ID NO: 49 - protein, 416 aa, Homo sapiens
GPLPKPTLWAEPGSVITQGSPVTLRCQGSLETQEYHLYREKKTALWITRIPQELVKKGQFPILSITWEHAGRYCCIYGSHTVGLSESSDPLELVVTGAYSKPTLSALPSPVVTSGGNVTIQCDSQVAFDGFILCKEGEDEHPQCLNSHSHARGSSRAIFSVGPVSPSRRWSYRCYGYDSRAPYVWSLPSDLLGLLVPGVSKKPSLSVQPGPVVAPGEKLTFQCGSDAGYDRFVLYKEWGRDFLQRPGRQPQAGLSQANFTLGPVSRSYGGQYTCSGAYNLSSEWSAPSDPLDILITGQIRARPFLSVRPGPTVASGENVTLLCQSQGGMHTFLLTKEGAADSPLRLKSKRQSHKYQAEFPMSPVTSAHAGTYRCYGSLSSNPYLLTHPSDPLELVVSGAAETLSPPQNKSDSKAGE

SEQ ID NO: 50 - protein, 673 aa, Synthetic construct
GHLPKPTLWAEPGSVITQGSPVTLRCQGGQETQEYRLYREKKTAPWITRIPQELVKKGQFPIPSITWEHTGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHGGGGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 51 - protein, 673 aa, Synthetic construct
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHGGGGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 52 - protein, 638 aa, Macaca mulatta
MTPILMVLICLGLSLGSRTRVQAGTFPKPTLWAEPGSMISKGSPVTLRCQGSLPVQDYRLQREKKTASWVRRIQQELVKKGYFPIASITSEHAGQYRCQYYSHSWWSEPSDPLELVVTGAYSKPTLSALPSPVVASGGNVTLQCDSQVAGGFVLCKEGEDEHPQCLNSQPHTRGSSRAVFSVGPVSPSRRWSYRCYGYDSRSPYVWSLPSDLLELLVPGVSKKPSLSVQPGPVVAPGDKLTLQCGSDAGYNRFALYKEGERDFLQRPGRQPQAGLSQANFLLDPVRRSHGGQYRCSGAHNLSSEWSAPSDPLDILIAGQIRGRPSLLVQPGPTVVSGENVTLLCQSSWQFHVFLLTQAGAADAHLHLRSMYKYPKYQAEFPMSPVTSAHAGTYRCYGSHSSDSYLLSIPSDPLELVVSGPSGGPSSPTTGPTSTCGPEDQPLTPTGSDPQSGLGRHLGVVTGVLVAFVLLLFLLLLLFLVLRHRRQGKRWTSAQRKADFQHPAGAVEPEPRDRGLQRRSSPAANTQEENLYAAVKDTQPEDGVELDSRSPHDEDPQAVTYARVKHSRPRREMASPPSPLSEEFLDTKDTQAAASEDPQDVTYAQLQSLTLRRETTEPPPSQEREPPVESSIYATLTIH

SEQ ID NO: 53 - protein, 668 aa, Macaca fascicularis
MHRGLLHPQSRAVGGDAMTPILTVLICLGLSLGPRTHVQAGILPKPMLWAEPDRVITQGSPVTLRCQGNLEALGYHLYRERKSASWITSIRPELVRKGQFPIPSITWEDAGRYRCQYYSHSWWSEHSDPLELVVTGAYSKPTLSALPSPVVASGGNVTLQCDSRVAFDGFILCKEGEDEHSQCLNSQPRTRGSSRAVFSVGPVSPSRRWSYRCYGYDSSFPYVWSLPSDLLELLVSGVSKKPSLSVQPGPVVAPGDKVTLQCGSDAGYDRFVLYKEGEREFLQRPGRQPQAGLSQANFTLGPVSHSHGGQYRCCGAHNLSSEWSAPSDPLDILISGQTHARPSLSVQPGPTVASGENVTLLCQSQGWMDTFFLTKEGAADAPLHLKSKRRSHIYQAEFPMGPVTSAHAGTYRCYGSFSSNPYLLTHPSEPLELVVSGPSGSPSPPLTGPTPTPAGPEDQPLTPTGSAPQSGLGRHLGVVTGILVAFVLLLFLLLLLFLVLRHQRQGKHWTSAQRKADFQHPAGAVEPEPRDRGFQRRSSPAADTQEENLYAAVKDTQPEDRVELDSRQSPHDEDPQAVTYAQVKHSGPRREMTSPPSPLSEEFLDTKDTQAEEDRQRDTEAAASEDPQDVTYAQLQSLTLRREATEPPPTQEREPPAEPSVYATLAIH

SEQ ID NO: 54 - protein, 119 aa, unidentified
DVQLQGSGPGLVKPSETLSLTCSVTGYSITSGYYWNWIRQFPGKKLEWMGYISYDGSNNYNPSLKNRITISRDTSKNQFSLKLNSVTAADTATYYCAHGYSYYYAMDAWGQGTSVTVSS

SEQ ID NO: 55 - protein, 106 aa, unidentified
DIQMTQSPSSLSASVGDRVTITCRTSQDISNYLNWYQQKPGKAVKLLISYTSRLHSGVPSRFSGSGSGTDYTLTISSLQPEDFATYYCQQGNTLPTFGQGTKLEIK

SEQ ID NO: 56 - protein, 448 aa, unidentified
EVQLQQWGAGLLKPSETLSLTCAVYGGSFSGYYWSWIRQPPGKGLEWIGEINHAGSTNYNPSLKSRVTISVDTSKNQFSLKLSSVTAADTAVYYCARLPTRWVTTRYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 217 aa, unidentified
ESVLTQPPSVSGAPGQRVTISCTGSSSNIGAGYDVHWYQQLPGTAPKLLIYGDSNRPSGVPDRFSVSKSGASASLAITGLQAEDEADYYCQSFDNSLSAYVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 58 - skipped

SEQ ID NO: 59 - protein, 651 aa, Homo sapiens [sequence omitted]

SEQ ID NO: 60 - protein, 597 aa, Homo sapiens
MTPIVTVLICLGLSLGPRTRVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVMAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 61 - protein, 439 aa, Homo sapiens
MTPILTVLICLGLSLDPRTHVQAGPLPKPTLWAEPGSVITQGSPVTLRCQGSLETQEYHLYREKKTALWITRIPQELVKKGQFPILSITWEHAGRYCCIYGSHTAGLSESSDPLELVVTGAYSKPTLSALPSPVVTSGGNVTIQCDSQVAFDGFILCKEGEDEHPQCLNSHSHARGSSRAIFSVGPVSPSRRWSYRCYGYDSRAPYVWSLPSDLLGLLVPGVSKKPSLSVQPGPVVAPGEKLTFQCGSDAGYDRFVLYKEWGRDFLQRPGRQPQAGLSQANFTLGPVSRSYGGQYTCSGAYNLSSEWSAPSDPLDILITGQIRARPFLSVRPGPTVASGENVTLLCQSQGGMHTFLLTKEGAADSPLRLKSKRQSHKYQAEFPMSPVTSAHAGTYRCYGSLSSNPYLLTHPSDPLELVVSGAAETLSPPQNKSDSKAGE

SEQ ID NO: 62 - protein, 489 aa, Homo sapiens
MTPIVTVLICLRLSLGPRTHVQAGTLPKPTLWAEPGSVITQGSPVTLWCQGILETQEYRLYREKKTAPWITRIPQEIVKKGQFPIPSITWEHTGRYRCFYGSHTAGWSEPSDPLELVVTGAYIKPTLSALPSPVVTSGGNVTLHCVSQVAFGSFILCKEGEDEHPQCLNSQPRTHGWSRAIFSVGPVSPSRRWSYRCYAYDSNSPHVWSLPSDLLELLVLGVSKKPSLSVQPGPIVAPGESLTLQCVSDVSYDRFVLYKEGERDFLQLPGPQPQAGLSQANFTLGPVSRSYGGQYRCSGAYNLSSEWSAPSDPLDILIAGQFRGRPFISVHPGPTVASGENVTLLCQSWGPFHTFLLTKAGAADAPLRLRSIHEYPKYQAEFPMSPVTSAHSGTYRCYGSLSSNPYLLSHPSDSLELMVSGAAETLSPPQNKSDSKAGAANTLSPSQNKTASHPQDYTVENLIRMGIAGLVLVVLGILLFEAQHSQRSL

SEQ ID NO: 63 - protein, 483 aa, Homo sapiens
MTPILTVLICLGLSLGPRTHVQAGHLPKPTLWAEPGSVIIQGSPVTLRCQGSLQAEEYHLYRENKSASWVRRIQEPGKNGQFPIPSITWEHAGRYHCQYYSHNHSSEYSDPLELVVTGAYSKPTLSALPSPVVTLGGNVTLQCVSQVAFDGFILCKEGEDEHPQRLNSHSHARGWSWAIFSVGPVSPSRRWSYRCYAYDSNSPYVWSLPSDLLELLVPGVSKKPSLSVQPGPMVAPGESLTLQCVSDVGYDRFVLYKEGERDFLQRPGWQPQAGLSQANFTLGPVSPSHGGQYRCYSAHNLSSEWSAPSDPLDILITGQFYDRPSLSVQPVPTVAPGKNVTLLCQSRGQFHTFLLTKEGAGHPPLHLRSEHQAQQNQAEFRMGPVTSAHVGTYRCYSSLSSNPYLLSLPSDPLELVVSEAAETLSPSQNKTDSTTTSLGQHPQDYTVENLIRMGVAGLVLVVLGILLFEAQHSQRSLQDAAGR

SEQ ID NO: 64 - protein, 631 aa, Homo sapiens
MTPALTALLCLGLSLGPRTRVQAGPFPKPTLWAEPGSVISWGSPVTIWCQGSQEAQEYRLHKEGSPEPLDRNNPLEPKNKARFSIPSMTEHHAGRYRCHYYSSAGWSEPSDPLEMVMTGAYSKPTLSALPSPVVASGGNMTLRCGSQKGYHHFVLMKEGEHQLPRTLDSQQLHSRGFQALFPVGPVTPSHRWRFTCYYYYTNTPWVWSHPSDPLEILPSGVSRKPSLLTLQGPVLAPGQSLTLQCGSDVGYNRFVLYKEGERDFLQRPGQQPQAGLSQANFTLGPVSPSNGGQYRCYGAHNLSSEWSAPSDPLNILMAGQIYDTVSLSAQPGPTVASGENVTLLCQSWWQFDTFLLTKEGAAHPPLRLRSMYGAHKYQAEFPMSPVTSAHAGTYRCYGSYSSNPHLLSHPSEPLELVVSGHSGGSSLPPTGPPSTPGLGRYLEVLIGVSVAFVLLLFLLLFLLLRRQRHSKHRTSDQRKTDFQRPAGAAETEPKDRGLLRRSSPAADVQEENLYAAVKDTQSEDRVELDSQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYATLAIH

SEQ ID NO: 65 - protein, 448 aa, Homo sapiens
MIPTFTALLCLGLSLGPRTHMQAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEDPRPSPTRSVSTAAGPEDQPLMPTGSVPHSGLRRHWEVLIGVLVVSILLLSLLLFLLLQHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYATLAIH

SEQ ID NO: 66 - protein, 590 aa, Homo sapiens
MTLTLSVLICLGLSVGPRTCVQAGTLPKPTLWAEPASVIARGKPVTLWCQGPLETEEYRLDKEGLPWARKRQNPLEPGAKAKFHIPSTVYDSAGRYRCYYETPAGWSEPSDPLELVATGFYAEPTLLALPSPVVASGGNVTLQCDTLDGLLTFVLVEEEQKLPRTLYSQKLPKGPSQALFPVGPVTPSCRWRFRCYYYYRKNPQVWSNPSDLLEILVPGVSRKPSLLIPQGSVVARGGSLTLQCRSDVGYDIFVLYKEGEHDLVQGSGQQPQAGLSQANFTLGPVSRSHGGQYRCYGAHNLSPRWSAPSDPLDILIAGLIPDIPALSVQPGPKVASGENVTLLCQSWHQIDTFFLTKEGAAHPPLCLKSKYQSYRHQAEFSMSPVTSAQGGTYRCYSAIRSYPYLLSSPSYPQELVVSGPSGDPSLSPTGSTPTPGPEDQPLTPTGLDPQSGLGRHLGVVTGVSVAFVLLLFLLLFLLLRHRHQSKHRTSAHFYRPAGAAGPEPKDQGLQKRASPVADIQEEILNAAVKDTQPKDGVEMDAPAAASEAPQDVTYAQLHSLTLRREATEPPPSQEREPPAEPSIYAPLAIH

SEQ ID NO: 67 - protein, 499 aa, Homo sapiens
MTLILTSLLFFGLSLGPRTRVQAENLLKPILWAEPGPVITWHNPVTIWCQGTLEAQGYRLDKEGNSMSRHILKTLESENKVKLSIPSMMWEHAGRYHCYYQSPAGWSEPSDPLELVVTAYSRPTLSALPSPVVTSGVNVTLRCASRLGLGRFTLIEEGDHRLSWTLNSHQHNHGKFQALFPMGPLTFSNRGTFRCYGYENNTPYVWSEPSDPLQLLVSGVSRKPSLLTLQGPVVTPGENLTLQCGSDVGYIRYTLYKEGADGLPQRPGRQPQAGLSQANFTLSPVSRSYGGQYRCYGAHNVSSEWSAPSDPLDILIAGQISDRPSLSVQPGPTVTSGEKVTLLCQSWDPMFTFLLTKEGAAHPPLRLRSMYGAHKYQAEFPMSPVTSAHAGTYRCYGSRSSNPYLLSHPSEPLELVVSGATETLNPAQKKSDSKTAPHLQDYTVENLIRMGVAGLVLLFLGILLFEAQHSQRSPPRCSQEANSRKDNAPFRVVEPWEQI

SEQ ID NO: 68 - protein, 299 aa, Homo sapiens
MAPWSHPSAQLQPVGGDAVSPALMVLLCLGLSLGPRTHVQAGNLSKATLWAEPGSVISRGNSVTIRCQGTLEAQEYRLVKEGSPEPWDTQNPLEPKNKARFSIPSMTEHHAGRYRCYYYSPAGWSEPSDPLELVVTGFYNKPTLSALPSPVVTSGENVTLQCGSRLRFDRFILTEEGDHKLSWTLDSQLTPSGQFQALFPVGPVTPSHRWMLRCYGSRRHILQVWSEPSDLLEIPVSGAADNLSPSQNKSDSGTASHLQDYAVENLIRMGMAGLILVVLGILIFQDWHSQRSPQAAAGR

SEQ ID NO: 69 - protein, 481 aa, Homo sapiens
MTPALTALLCLGLSLGPRTRVQAGPFPKPTLWAEPGSVISWGSPVTIWCQGSLEAQEYQLDKEGSPEPLDRNNPLEPKNKARFSIPSMTQHHAGRYRCHYYSSAGWSEPSDPLELVMTGFYNKPTLSALPSPVVASGGNMTLRCGSQKGYHHFVLMKEGEHQLPRTLDSQQLHSGGFQALFPVGPVTPSHRWRFTCYYYYTNTPRVWSHPSDPLEILPSGVSRKPSLLTLQGPVLAPGQSLTLQCGSDVGYDRFVLYKEGERDFLQRPGQQPQAGLSQANFTLGPVSPSHGGQYRCYGAHNLSSEWSAPSDPLNILMAGQIYDTVSLSAQPGPTVASGENVTLLCQSRGYFDTFLLTKEGAAHPPLRLRSMYGAHKYQAEFPMSPVTSAHAGTYRCYGSYSSNPHLLSFPSEPLELMVSGHSGGSSLPPTGPPSTPASHAKDYTVENLIRMGMAGLVLVFLGILLFEAQHSQRNPQDAAGR

SEQ ID NO: 70 - protein, 125 aa, unidentified
QVQLVQSGAEVKKPGASVKVSCKASGYTFTDYYINWVRQAPGQGLEWMGNVNPNDGGTTYNQKFKGRVTMTTDTSTSTAYMELRSLRSDDTAVYYCARREIYFYGTIYYYAMDYWGQGTLVTVSS

SEQ ID NO: 71 - protein, 111 aa, unidentified
DIQLTQSPSFLSASVGDRVTITCRASESVDYYGNSFMYWYQQKPGKAPKLLIYFASNLESGVPSRFSGSGSGTEFTLTISSLQPEDFATYYCQQNNEDPWTFGGGTKVEIK

SEQ ID NO: 72 - protein, 118 aa, unidentified
QVQLKESGPGLVAPSQSLSITCTVSGFSLTSYGVSWVRQPPGKGLEWLGVIWGDGSTNYHSALISRLSISKDNSKSQVFLKLNSLQTDDTATYYCAKPRWDDYAMDYWGQGTSVTVSS

SEQ ID NO: 73 - protein, 106 aa, unidentified
DIQMTQTTSSLSASLGDRVTISCRASQDISNYLNWYQQKPDGTVKLLIYYTSRLHSGVPSRFSGSGSGTDYSLTISNLEQEDIATYFCQQGNTLWTFGGGTKLEIK

SEQ ID NO: 74 - protein, 122 aa, unidentified
QVQLQQPGAELVKPGASVRMSCKASGYTFTSYWVHWVKQRPGQGLEWIGVIDPSDSYTSYNQNFKGKATLTVDTSSKTAYIHLSSLTSEDSAVYFCARGERYDGDYFAMDYWGQGTSVTVSS

SEQ ID NO: 75 - protein, 107 aa, unidentified
DIVMTQSPASLSVSVGETVTITCRASENIYSNLAWYQQKQGKSPQLLVYAATNLADGVPSRFSGSRSGTQYSLKINSLQSEDFGTYYCQHFWNTPRTFGGGTKLEIK

SEQ ID NO: 76 - protein, 117 aa, unidentified
QVQLQQSGAELVKPGASVRLSCKASGYTFTAHTIHWVKQRSGQGLEWIGWLYPGSGSIKYNEKFKDKATLTADKSSSTVYMELSRLTSEDSAVYFCARHTNWDFDYWGQGTTLTVSS

SEQ ID NO: 77 - protein, 111 aa, unidentified
NIVLTQSPASLAVSLGQRATISCKASQSVDYGGASYMNWYQQKPGQPPKLLIYAASNLESGIPARFSGSGSGTDLTLNIHPVEEEDAAMYYCQQSNEEPWTFGGGTKLEIK

SEQ ID NO: 78 - protein, 15 aa, Homo sapiens
AEFPMGPVTSAHAGT

SEQ ID NO: 79 - protein, 10 aa, Homo sapiens
LTHPSDPLEL

SEQ ID NO: 80 - protein, 11 aa, Homo sapiens
FVLYKDGERDF

SEQ ID NO: 81 - protein, 14 aa, Homo sapiens
GYDRFVLYKEGERD

SEQ ID NO: 82 - protein, 13 aa, Homo sapiens
YDRFVLYKEWGRD

SEQ ID NO: 83 - protein, 12 aa, Homo sapiens
SSEWSAPSDPLD

SEQ ID NO: 84 - protein, 11 aa, Homo sapiens
ECSAPSDPLDI

SEQ ID NO: 85 - protein, 11 aa, Homo sapiens
SEWSAPSDPLD

SEQ ID NO: 86 - protein, 10 aa, Homo sapiens
LQCVSDVGYD

SEQ ID NO: 87 - protein, 12 aa, Homo sapiens
FQCGSDAGYDRF

SEQ ID NO: 88 - protein, 13 aa, Homo sapiens
FLLTKEGAADDPW

SEQ ID NO: 89 - protein, 14 aa, Homo sapiens
AADAPLRLRSIHEY

SEQ ID NO: 90 - protein, 8 aa, Homo sapiens
RSYGGQYR

SEQ ID NO: 91 - protein, 12 aa, Homo sapiens
PVSRSYGGQYRC

SEQ ID NO: 92 - protein, 11 aa, Homo sapiens
LDILIAGQFYD

SEQ ID NO: 93 - protein, 10 aa, Homo sapiens
APSDPLDILI

SEQ ID NO: 94 - protein, 9 aa, Homo sapiens
PSDPLDILI

SEQ ID NO: 95 - protein, 423 aa, Homo sapiens
ENLLKPILWAEPGPVITWHNPVTIWCQGTLEAQGYRLDKEGNSMSRHILKTLESENKVKLSIPSMMWEHAGRYHCYYQSPAGWSEPSDPLELVVTAYSRPTLSALPSPVVTSGVNVTLRCASRLGLGRFTLIEEGDHRLSWTLNSHQHNHGKFQALFPMGPLTFSNRGTFRCYGYENNTPYVWSEPSDPLQLLVSGVSRKPSLLTLQGPVVTPGENLTLQCGSDVGYIRYTLYKEGADGLPQRPGRQPQAGLSQANFTLSPVSRSYGGQYRCYGAHNVSSEWSAPSDPLDILIAGQISDRPSLSVQPGPTVTSGEKVTLLCQSWDPMFTFLLTKEGAAHPPLRLRSMYGAHKYQAEFPMSPVTSAHAGTYRCYGSRSSNPYLLSHPSEPLELVVSGATETLNPAQKKSDSKTAPHLQDYTVEN

SEQ ID NO: 96 - protein, 227 aa, Homo sapiens
GNLSKATLWAEPGSVISRGNSVTIRCQGTLEAQEYRLVKEGSPEPWDTQNPLEPKNKARFSIPSMTEHHAGRYRCYYYSPAGWSEPSDPLELVVTGFYNKPTLSALPSPVVTSGENVTLQCGSRLRFDRFILTEEGDHKLSWTLDSQLTPSGQFQALFPVGPVTPSHRWMLRCYGSRRHILQVWSEPSDLLEIPVSGAADNLSPSQNKSDSGTASHLQDYAVENLIR

SEQ ID NO: 97 - protein, 424 aa, Homo sapiens
GPFPKPTLWAEPGSVISWGSPVTIWCQGSLEAQEYQLDKEGSPEPLDRNNPLEPKNKARFSIPSMTQHHAGRYRCHYYSSAGWSEPSDPLELVMTGFYNKPTLSALPSPVVASGGNMTLRCGSQKGYHHFVLMKEGEHQLPRTLDSQQLHSGGFQALFPVGPVTPSHRWRFTCYYYYTNTPRVWSHPSDPLEILPSGVSRKPSLLTLQGPVLAPGQSLTLQCGSDVGYDRFVLYKEGERDFLQRPGQQPQAGLSQANFTLGPVSPSHGGQYRCYGAHNLSSEWSAPSDPLNILMAGQIYDTVSLSAQPGPTVASGENVTLLCQSRGYFDTFLLTKEGAAHPPLRLRSMYGAHKYQAEFPMSPVTSAHAGTYRCYGSYSSNPHLLSFPSEPLELMVSGHSGGSSLPPTGPPSTPASHAKDYTVEN

SEQ ID NO: 98 - protein, 627 aa, Homo sapiens
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 99 - protein, 437 aa, Homo sapiens
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVMAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPGPEDQPLTPTGSDPQSGLGRHL

SEQ ID NO: 100 - protein, 420 aa, Homo sapiens
GPFPKPTLWAEPGSVISWGSPVTIWCQGSQEAQEYRLHKEGSPEPLDRNNPLEPKNKARFSIPSMTEHHAGRYRCHYYSSAGWSEPSDPLEMVMTGAYSKPTLSALPSPVVASGGNMTLRCGSQKGYHHFVLMKEGEHQLPRTLDSQQLHSRGFQALFPVGPVTPSHRWRFTCYYYYTNTPWVWSHPSDPLEILPSGVSRKPSLLTLQGPVLAPGQSLTLQCGSDVGYNRFVLYKEGERDFLQRPGQQPQAGLSQANFTLGPVSPSNGGQYRCYGAHNLSSEWSAPSDPLNILMAGQIYDTVSLSAQPGPTVASGENVTLLCQSWWQFDTFLLTKEGAAHPPLRLRSMYGAHKYQAEFPMSPVTSAHAGTYRCYGSYSSNPHLLSHPSEPLELVVSGHSGGSSLPPTGPPSTPGLGRYLE

SEQ ID NO: 101 - protein, 241 aa, Homo sapiens
PRTHMQAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEDPRPSPTRSVSTAAGPEDQPLMPTGSVPHSGLRRH

SEQ ID NO: 102 - protein, 439 aa, Homo sapiens
RTCVQAGTLPKPTLWAEPASVIARGKPVTLWCQGPLETEEYRLDKEGLPWARKRQNPLEPGAKAKFHIPSTVYDSAGRYRCYYETPAGWSEPSDPLELVATGFYAEPTLLALPSPVVASGGNVTLQCDTLDGLLTFVLVEEEQKLPRTLYSQKLPKGPSQALFPVGPVTPSCRWRFRCYYYYRKNPQVWSNPSDLLEILVPGVSRKPSLLIPQGSVVARGGSLTLQCRSDVGYDIFVLYKEGEHDLVQGSGQQPQAGLSQANFTLGPVSRSHGGQYRCYGAHNLSPRWSAPSDPLDILIAGLIPDIPALSVQPGPKVASGENVTLLCQSWHQIDTFFLTKEGAAHPPLCLKSKYQSYRHQAEFSMSPVTSAQGGTYRCYSAIRSYPYLLSSPSYPQELVVSGPSGDPSLSPTGSTPTPGPEDQPLTPTGLDPQSGLGRH

SEQ ID NO: 103 - protein, 450 aa, unidentified
QITLKESGPTLVKPTQTLTLTCTFSGFSLNTYAMGVSWIRQPPGKALEWLASIWWNGNKYNNPSLKSRLTVTKDTSKNQVVLTMTNMDPVDTATYYCAHSRIIRFTDYVMDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 104 - protein, 214 aa, unidentified
DIQMTQSPSSLSTSVGDRVTITCRASEDIYNDLAWYQQKPGKAPKLLIYNANSLHTGVASRFSGSGSGTDFTFTISSLQPEDVATYFCQQYYDYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 20 aa, Homo sapiens
FKDPNAPKRLPSAFFLFCSE